SEQUENCE LISTING (189 sequences)

SEQ ID NO: 1 - DNA, 361 bp, synthetic construct
  source 1..361: mol_type = other DNA
caggtccaactgctgcagcctggggctgagctggtgaggcctggggcctcagtgaggctgtcctgcaagacttctggctacaccttcaccagttactggataaactgggtgaaacagaggcctggacaaggccttgagtggatcggaaaaatttttccttctgacagtcatactaattacaatcaaaaattcaaggacaaggccacattgactgtagacaaatcctccagcacagcctacatgcagctcatcagcccgacatctgaggactctgcggtctattactgtacgagggacttcgatacccagttctatgctatggaatactggggtcagggaacctcagtcaccgtctcctcag

SEQ ID NO: 2 - DNA, 24 bp, synthetic construct
  source 1..24: mol_type = other DNA
ggctacaccttcaccagttactgg

SEQ ID NO: 3 - DNA, 24 bp, synthetic construct
  source 1..24: mol_type = other DNA
atttttccttctgacagtcatact

SEQ ID NO: 4 - DNA, 41 bp, synthetic construct
  source 1..41: mol_type = other DNA
tacgagggacttcgatacccagtctcatgctatggaatact

SEQ ID NO: 5 - DNA, 13 bp, synthetic construct
  source 1..13: mol_type = other DNA
ctggataaactgg

SEQ ID NO: 6 - DNA, 46 bp, synthetic construct
  source 1..46: mol_type = other DNA
atttttccttctgacagtcatactaattacaatcaaaaattcaagg

SEQ ID NO: 7 - DNA, 30 bp, synthetic construct
  source 1..30: mol_type = other DNA
tcgatacccagttctatgctatggaatact

SEQ ID NO: 8 - DNA, 22 bp, synthetic construct
  source 1..22: mol_type = other DNA
ctacaccttcaccagttactgg

SEQ ID NO: 9 - DNA, 15 bp, synthetic construct
  source 1..15: mol_type = other DNA
ccttctgacagtcat

SEQ ID NO: 10 - DNA, 39 bp, synthetic construct
  source 1..39: mol_type = other DNA
acttctggctacaccttcaccagttactggataaactgg

SEQ ID NO: 11 - DNA, 28 bp, synthetic construct
  source 1..28: mol_type = other DNA
aatttttccttctgacagtcatactaat

SEQ ID NO: 12 - DNA, 318 bp, synthetic construct
  source 1..318: mol_type = other DNA
caaattgttctcacccagtctccaacaatcatgtctgcatctccagggcagaaagtcaccataaactgcagtgccatctcaagtgtaaattgcatgcactggtaccagcagaagccaggatcctcccccaaactctggatttatgcaacatccaaactgactcttggagtccctgcttgcttcagtggcagtgggtctgggacctcttactctctcacagtcaacagcatggtgactgaagatgccacctcttatttctgtcatcagtggagtagttacccaccaacgttcggtgctgggaccaagctggagctgaaa

SEQ ID NO: 13 - skipped

SEQ ID NO: 14 - DNA, 13 bp, synthetic construct
  source 1..13: mol_type = other DNA
ttatgcaacatcc

SEQ ID NO: 15 - DNA, 27 bp, synthetic construct
  source 1..27: mol_type = other DNA
catcagtggagtagttacccaccaacg

SEQ ID NO: 16 - DNA, 13 bp, synthetic construct
  source 1..13: mol_type = other DNA
gcactggtaccag

SEQ ID NO: 17 - DNA, 49 bp, synthetic construct
  source 1..49: mol_type = other DNA
atccaaactgactcttggagtccctgcttgcttcagtggcagtgggtct

SEQ ID NO: 18 - DNA, 22 bp, synthetic construct
  source 1..22: mol_type = other DNA
ctcaagtgtaaattgcatgcac

SEQ ID NO: 19 - DNA, 15 bp, synthetic construct
  source 1..15: mol_type = other DNA
ctgactcttggagtc

SEQ ID NO: 20 - DNA, 39 bp, synthetic construct
  source 1..39: mol_type = other DNA
agtgccatctcaagtgtaaattgcatgcactggtaccag

SEQ ID NO: 21 - DNA, 28 bp, synthetic construct
  source 1..28: mol_type = other DNA
atccaaactgactcttggagtccctgct

SEQ ID NO: 22 - DNA, 360 bp, synthetic construct
  source 1..360: mol_type = other DNA
caggtgcagctgcttcagccaggggcagaacttgtacggcccggcgcaagcgtacgcttgtcctgcaaaacgagtggatatacgtttacctcctactggatcaactgggttaaacagagaccgggtcaaggtcttgagtggatcggtaagattttccccagtgattcccacactaactataatcaaaaatttaaggataaagctacgttgacggtcgataaatcatcttccaccgcgtatatgcaactgatatctccgaccagtgaggattctgccgtttactattgcacaagagatttcgacacgcagttctacgctatggaatactggggtcaggggacttctgtaacggtaagctct

SEQ ID NO: 23 - DNA, 24 bp, synthetic construct
  source 1..24: mol_type = other DNA
attttccccagtgattcccacact

SEQ ID NO: 24 - DNA, 12 bp, synthetic construct
  source 1..12: mol_type = other DNA
tggatcaactgg

SEQ ID NO: 25 - DNA, 46 bp, synthetic construct
  source 1..46: mol_type = other DNA
attttccccagtgattcccacactaactataatcaaaaatttaagg

SEQ ID NO: 26 - DNA, 36 bp, synthetic construct
  source 1..36: mol_type = other DNA
gagatttcgacacgcagttctacgctatggaatact

SEQ ID NO: 27 - DNA, 21 bp, synthetic construct
  source 1..21: mol_type = other DNA
tatacgtttacctcctactgg

SEQ ID NO: 28 - DNA, 15 bp, synthetic construct
  source 1..15: mol_type = other DNA
cccagtgattcccac

SEQ ID NO: 29 - DNA, 39 bp, synthetic construct
  source 1..39: mol_type = other DNA
acgagtggatatacgtttacctcctactggatcaactgg

SEQ ID NO: 30 - DNA, 27 bp, synthetic construct
  source 1..27: mol_type = other DNA
attttccccagtgattcccacactaac

SEQ ID NO: 31 - DNA, 318 bp, synthetic construct
  source 1..318: mol_type = other DNA
caaattgtcctgacacaaagccctacaataatgagtgcatcaccgggacaaaaggttacaattaactgctgtgccattagtagcgtgaattgcatgcactggtatcagcagaagcccggttcttcacctaaactttggatatatgctactagtaagctgaccctcggagtaccggcttgttttagtggaagtggatcagggaccagttacagcttgacggttaattcaatggttacagaagatgctacttcctatttttgccaccagtggtcttcttatcctcctacattcggggctggaactaaattggagttgaag

SEQ ID NO: 32 - DNA, 13 bp, synthetic construct
  source 1..13: mol_type = other DNA
atatgctactagt

SEQ ID NO: 33 - DNA, 27 bp, synthetic construct
  source 1..27: mol_type = other DNA
caccagtggtcttcttatcctcctaca

SEQ ID NO: 34 - DNA, 30 bp, synthetic construct
  source 1..30: mol_type = other DNA
tgtgccatctcaagtgtaaattacatgcac

SEQ ID NO: 35 - DNA, 47 bp, synthetic construct
  source 1..47: mol_type = other DNA
tagtaagctgaccctcggagtaccggcttgttttagtggaagtggat

SEQ ID NO: 36 - skipped

SEQ ID NO: 37 - DNA, 16 bp, synthetic construct
  source 1..16: mol_type = other DNA
gctgaccctcggagta

SEQ ID NO: 38 - skipped

SEQ ID NO: 39 - DNA, 28 bp, synthetic construct
  source 1..28: mol_type = other DNA
tagtaagctgaccctcggagtaccggct

SEQ ID NO: 40 - protein, 120 aa, synthetic construct
QVQLLQPGAELVRPGASVRLSCKTSGYTFTSYWINWVKQRPGQGLEWIGKIFPSDSHTNYNQKFKDKATLTVDKSSSTAYMQLISPTSEDSAVYYCTRDFDTQFYAMEYWGQGTSVTVSS

SEQ ID NO: 41 - protein, 8 aa, synthetic construct
GYTFTSYW

SEQ ID NO: 42 - protein, 8 aa, synthetic construct
IFPSDSHT

SEQ ID NO: 43 - protein, 13 aa, synthetic construct
TRDFDTQFYAMEY

SEQ ID NO: 44 - protein, 5 aa, synthetic construct
SYWIN

SEQ ID NO: 45 - protein, 17 aa, synthetic construct
KIFPSDSHTNYNQKFKD

SEQ ID NO: 46 - protein, 11 aa, synthetic construct
DFDTQFYAMEY

SEQ ID NO: 47 - protein, 7 aa, synthetic construct
GYTFTSY

SEQ ID NO: 48 - protein, 6 aa, synthetic construct
FPSDSH

SEQ ID NO: 49 - protein, 13 aa, synthetic construct
KTSGYTFTSYWIN

SEQ ID NO: 50 - protein, 10 aa, synthetic construct
KIFPSDSHTN

SEQ ID NO: 51 - skipped

SEQ ID NO: 52 - protein, 106 aa, synthetic construct
QIVLTQSPTIMSASPGQKVTINCSAISSVNCMHWYQQKPGSSPKLWIYATSKLTLGVPACFSGSGSGTSYSLTVNSMVTEDATSYFCHQWSSYPPTFGAGTKLELK

SEQ ID NO: 53 - protein, 7 aa, synthetic construct
AISSVNC

SEQ ID NO: 54 - protein, 9 aa, synthetic construct
HQWSSYPPT

SEQ ID NO: 55 - protein, 10 aa, synthetic construct
SAISSVNCMH

SEQ ID NO: 56 - protein, 7 aa, synthetic construct
ATSKLTL

SEQ ID NO: 57 - skipped

SEQ ID NO: 58 - protein, 8 aa, synthetic construct
YATSKLTL

SEQ ID NO: 59 - DNA, 353 bp, synthetic construct
  source 1..353: mol_type = other DNA
caggtccaactgctgcagcctggggctgagctggtgaggcctggggcctcagtgaggctgtcctgcaagacttctggctacaccttcaccagttactggataaactgggtgaaacagaggcctggacaaggccttgagtggatcggaaaaatttttccttctgacagtcatactaattacaatcaaaaattcaaggacaaggccacattgactgtagacaaatcctccagcacagcctacatgcagctcatcagcccgacatctgaggactctgcggtctattactgtacgagggacttcgatacccagttctatgctatggaatactggggtcagggaacctcagtcaccgt

SEQ ID NO: 60 - DNA, 353 bp, synthetic construct
  source 1..353: mol_type = other DNA
caggtgcagctgcttcagccaggggcagaacttgtacggcccggcgcaagcgtacgcttgtcctgcaaaacgagtggatatacgtttacctcctactggatcaactgggttaaacagagaccgggtcaaggtcttgagtggatcggtaagattttccccagtgattcccacactaactataatcaaaaatttaaggataaagctacgttgacggtcgataaatcatcttccaccgcgtatatgcaactgatatctccgaccagtgaggattctgccgtttactattgcacaagagatttcgacacgcagttctacgctatggaatactggggtcaggggacttctgtaacggt

SEQ ID NO: 61 - DNA, 317 bp, synthetic construct
  source 1..317: mol_type = other DNA
caaattgttctcacccagtctccaacaatcatgtctgcatctccagggcagaaagtcaccataaactgcagtgccatctcaagtgtaaattgcatgcactggtaccagcagaagccaggatcctcccccaaactctggatttatgcaacatccaaactgactcttggagtccctgcttgcttcagtggcagtgggtctgggacctcttactctctcacagtcaacagcatggtgactgaagatgccacctcttatttctgtcatcagtggagtagttacccaccaacgttcggtgctgggaccaagctggagctgaa

SEQ ID NO: 62 - DNA, 317 bp, synthetic construct
  source 1..317: mol_type = other DNA
caaattgtcctgacacaaagccctacaataatgagtgcatcaccgggacaaaaggttacaattaactgctctgcgattagtagcgtgaattgcatgcactggtatcagcagaagcccggttcttcacctaaactttggatatatgctactagtaagctgaccctcggagtaccggcttgttttagtggaagtggatcagggaccagttacagcttgacggttaattcaatggttacagaagatgctacttcctatttttgccaccagtggtcttcttatcctcctacattcggggctggaactaaattggagttgaa

SEQ ID NO: 63 - protein, 241 aa, synthetic construct
QIVLTQSPTIMSASPGQKVTINCSAISSVNCMHWYQQKPGSSPKLWIYATSKLTLGVPACFSGSGSGTSYSLTVNSMVTEDATSYFCHQWSSYPPTFGAGTKLELKGGGGSGGGGSGGGGSQVQLLQPGAELVRPGASVRLSCKTSGYTFTSYWINWVKQRPGQGLEWIGKIFPSDSHTNYNQKFKDKATLTVDKSSSTAYMQLISPTSEDSAVYYCTRDFDTQFYAMEYWGQGTSVTVSS

SEQ ID NO: 64 - protein, 15 aa, synthetic construct
GGGGSGGGGSGGGGS

SEQ ID NO: 65 - skipped

SEQ ID NO: 66 - protein, 15 aa, synthetic construct
GSDSNAGHASAGNTS

SEQ ID NO: 67-69 - skipped (3 entries)

SEQ ID NO: 70 - DNA, 23 bp, synthetic construct
  source 1..23: mol_type = other DNA
caaattgttctcacccagtctcc

SEQ ID NO: 71 - DNA, 29 bp, synthetic construct
  source 1..29: mol_type = other DNA
tttcagctccagcttggtcccagcaccga

SEQ ID NO: 72 - DNA, 23 bp, synthetic construct
  source 1..23: mol_type = other DNA
caggtccaactgctgcagcctgg

SEQ ID NO: 73 - DNA, 27 bp, synthetic construct
  source 1..27: mol_type = other DNA
acagtaatagaccgcagagtcctcaga

SEQ ID NO: 74 - protein, 617 aa, Measles morbillivirus
MSPQRDRINAFYKDNPHPKGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLSRGYNVSSIVTMTSQGMYGGTYLVEKPNLSSKGSELSQLSMYRVFEVGVIRNPGLGAPVFHMTNYFEQPVSNDLSNCMVALGELKLAALCHGGDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGEDCHAPTYLPAEVDGDVKLSSNLVILPGQDLQYVLATYDTSRVEHAVVYYVYSPSRSFSYFYPFRLPIKGIPIELQVECFTWDQKLWCRHFCVLADSESGGHITHSGMVGMGVSCTVTREDGTNSR

SEQ ID NO: 75 - skipped

SEQ ID NO: 76 - DNA, 102 bp, synthetic construct
  source 1..102: mol_type = other DNA
atgaacagagaacatcttatgattgatagaccttatgttttgctggctgttctgtttgtcatgtttctgagcttgatcgggttgctagccattgcaggcatt

SEQ ID NO: 77 - DNA, 1670 bp, synthetic construct
  source 1..1670: mol_type = other DNA
agacttcatcgggcagccatctacaccgcagagatccataaaagcctcagcaccaatctagatgtaactactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcggagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaat

SEQ ID NO: 78 - DNA, 45 bp, synthetic construct
  source 1..45: mol_type = other DNA
ggggggggggggagtggggggggggggagtggggggggggggagt

SEQ ID NO: 79 - protein, 5 aa, synthetic construct
  REPEAT 1..5: note = Sequence can be repeated n times, wherein n is an integer of 1, 2, 3, 4, 5, 6, 7, 8, 9 or 10
GGGGS

SEQ ID NO: 80 - protein, 9 aa, synthetic construct
  REPEAT 1..4: note = Sequence can be repeated n times, wherein n is an integer of 1, 2, 3, 4, 5, 6, 7, 8, 9 or 10
  REPEAT 5..9: note = Sequence can be repeated n times, wherein n is an integer of 1, 2, 3, 4, 5, 6, 7, 8, 9 or 10
GGGSGGGGS

SEQ ID NO: 81 - protein, 7 aa, synthetic construct
  REPEAT 1..4: note = Sequence can be repeated n times, wherein n is an integer of 1, 2, 3, 4, 5, 6, 7, 8, 9 or 10
  REPEAT 5..7: note = Sequence can be repeated n times, wherein n is an integer of 1, 2, 3, 4, 5, 6, 7, 8, 9 or 10
GGGSGGS

SEQ ID NO: 82 - protein, 9 aa, synthetic construct
  REPEAT 1..4: note = Sequence can be repeated n times, wherein n is an integer of 1, 2, 3, 4, 5, 6, 7, 8, 9 or 10
GGGSGGGGS

SEQ ID NO: 83 - protein, 20 aa, synthetic construct
GGGGSGGGGSGGGGSGGGGS

SEQ ID NO: 84 - protein, 10 aa, synthetic construct
GGGGSGGGGS

SEQ ID NO: 85 - protein, 5 aa, synthetic construct
GGGGS

SEQ ID NO: 86 - protein, 8 aa, synthetic construct
GGGSGGGS

SEQ ID NO: 87 - protein, 4 aa, synthetic construct
GGGS

SEQ ID NO: 88 - protein, 6 aa, synthetic construct
GGSGGS

SEQ ID NO: 89 - protein, 11 aa, synthetic construct
GGSGGGSGGSG

SEQ ID NO: 90 - protein, 10 aa, synthetic construct
GGSGGGSGSG

SEQ ID NO: 91 - protein, 8 aa, synthetic construct
GGSGGGSG

SEQ ID NO: 92 - protein, 10 aa, synthetic construct
SASSSVSYMN

SEQ ID NO: 93 - protein, 11 aa, synthetic construct
RWIYDTSKLAS

SEQ ID NO: 94 - protein, 9 aa, synthetic construct
QQWSSNPFT

SEQ ID NO: 95 - protein, 13 aa, synthetic construct
KASGYTFTRYTMH

SEQ ID NO: 96 - protein, 16 aa, synthetic construct
INPSRGYTNYNQKFKD

SEQ ID NO: 97 - protein, 10 aa, synthetic construct
YYDDHYCLDY

SEQ ID NO: 98 - protein, 242 aa, synthetic construct
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 99 - protein, 11 aa, synthetic construct
QSLVHNNGNTY

SEQ ID NO: 100 - protein, 9 aa, synthetic construct
GQGTQYPFT

SEQ ID NO: 101 - protein, 8 aa, synthetic construct
GFTFTKAW

SEQ ID NO: 102 - protein, 10 aa, synthetic construct
IKDKSNSYAT

SEQ ID NO: 103 - protein, 12 aa, synthetic construct
RGVYYALSPFDY

SEQ ID NO: 104 - protein, 11 aa, synthetic construct
QSLVHDNGNTY

SEQ ID NO: 105 - protein, 8 aa, synthetic construct
GFTFSNAW

SEQ ID NO: 106 - protein, 10 aa, synthetic construct
IKARSNNYAT

SEQ ID NO: 107 - protein, 12 aa, synthetic construct
RGTYYASKPFDY

SEQ ID NO: 108 - protein, 11 aa, synthetic construct
QSLEHNNGNTY

SEQ ID NO: 109 - protein, 10 aa, synthetic construct
IKDKSNNYAT

SEQ ID NO: 110 - protein, 13 aa, synthetic construct
RYVHYGIGYAMDA

SEQ ID NO: 111 - protein, 9 aa, synthetic construct
GQGTHYPFT

SEQ ID NO: 112 - protein, 8 aa, synthetic construct
GFTFTNAW

SEQ ID NO: 113 - protein, 9 aa, synthetic construct
KDKSNNYAT

SEQ ID NO: 114 - protein, 13 aa, synthetic construct
RYVHYRFAYALDA

SEQ ID NO: 115 - protein, 120 aa, synthetic construct
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 116 - protein, 107 aa, synthetic construct
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 117 - protein, 11 aa, synthetic construct
HASQNIYVWLN

SEQ ID NO: 118 - protein, 7 aa, synthetic construct
KASNLHT

SEQ ID NO: 119 - protein, 9 aa, synthetic construct
QQGQTYPYT

SEQ ID NO: 120 - protein, 10 aa, synthetic construct
GYTFTSYYIH

SEQ ID NO: 121 - protein, 14 aa, synthetic construct
CIYPGNVNTNYNEK

SEQ ID NO: 122 - protein, 11 aa, synthetic construct
SHYGLDWNFDV

SEQ ID NO: 123 - protein, 5 aa, synthetic construct
SYYIH

SEQ ID NO: 124 - protein, 17 aa, synthetic construct
CIYPGNVNTNYNEKFKD

SEQ ID NO: 125 - protein, 7 aa, synthetic construct
RASQSVS

SEQ ID NO: 126 - protein, 6 aa, synthetic construct
ASNRAT

SEQ ID NO: 127 - protein, 10 aa, synthetic construct
QRSNWPPALT

SEQ ID NO: 128 - protein, 4 aa, synthetic construct
YYWS

SEQ ID NO: 129 - protein, 12 aa, synthetic construct
YGPGNYDWYFDL

SEQ ID NO: 130 - protein, 11 aa, synthetic construct
SGDNIGDQYAH

SEQ ID NO: 131 - protein, 7 aa, synthetic construct
QDKNRPS

SEQ ID NO: 132 - protein, 11 aa, synthetic construct
ATYTGFGSLAV

SEQ ID NO: 133 - protein, 10 aa, synthetic construct
GYSFSTYWIS

SEQ ID NO: 134 - protein, 14 aa, synthetic construct
KIYPGDSYTNYSPS

SEQ ID NO: 135 - protein, 7 aa, synthetic construct
GYGIFDY

SEQ ID NO: 136 - protein, 11 aa, synthetic construct
RTSRSISQYLA

SEQ ID NO: 137 - protein, 7 aa, synthetic construct
SGSTLQS

SEQ ID NO: 138 - protein, 9 aa, synthetic construct
QQHNENPLT

SEQ ID NO: 139 - protein, 6 aa, synthetic construct
GFNIKD

SEQ ID NO: 140 - protein, 9 aa, synthetic construct
RIDPANDNT

SEQ ID NO: 141 - protein, 9 aa, synthetic construct
GYGYYVFDH

SEQ ID NO: 142 - protein, 109 aa, synthetic construct
EIVLTQSPVTLSLSPGERATLSCRASQSVSSYLAWYQQKPGQAPRLLIYDASNRATGIPARFSGSGSGTDFTLTISSLEPEDFAVYYCQQRSNWMYTFGQGTKLEIKRT

SEQ ID NO: 143 - protein, 123 aa, synthetic construct
QVQLVQSGAEVKKPGSSVKVSCKASGGTFSFYAISWVRQAPGQGLEWMGGFIPIFGAANYAQKFQGRVTITADESTSTAYMELSSLRSDDTAVYYCARIPSGSYYYDYDMDVWGQGTTVTVSS

SEQ ID NO: 144 - protein, 318 aa, synthetic construct
CAAATTGTCCTGACACAAAGCCCTACAATAATGAGTGCATCACCGGGACAAAAGGTTACAATTAACTGCTCTGCCATTAGTAGCGTGAATTACATGCACTGGTATCAGCAGAAGCCCGGTTCTTCACCTAAACTTTGGATATATGCTACTAGTAAGCTGACCCTCGGAGTACCGGCTCGTTTTAGTGGAAGTGGATCAGGGACCAGTTACAGCTTGACGGTTAATTCAATGGTTACAGAAGATGCTACTTCCTATTTTTGCCACCAGTGGTCTTCTTATCCTCCTACATTCGGGGCTGGAACTAAATTGGAGTTGAAG

SEQ ID NO: 145 - protein, 106 aa, synthetic construct
QIVLTQSPTIMSASPGQKVTINCSAISSVNYMHWYQQKPGSSPKLWIYATSKLTLGVPARFSGSGSGTSYSLTVNSMVTEDATSYFCHQWSSYPPTFGAGTKLELK

SEQ ID NO: 146 - protein, 60 aa, synthetic construct
QIVLTQSPTIMSASPGQKVTINCSAISSVNCMHWYQQKPGSSPKLWIYATSKLTLGVPAC

SEQ ID NO: 147 - protein, 60 aa, synthetic construct
QIVLTQSPAIMSASPGEKVTMTCSASSSVSYMHWYQQKSGTSPKRWIYDTSKLASGVPAR

SEQ ID NO: 148 - protein, 60 aa, synthetic construct
DIVLTQSPAIMSASPGEKVTMTCSASSSVSYMHWYQQKSGTSPKRWIYDTSKLASGVPAR

SEQ ID NO: 149 - protein, 60 aa, synthetic construct
QIVLTQSPTIMSASPGEKVTMTCSASSSVDYMHWYQQKSGTSPKRWIYDTYKLASGVPAR

SEQ ID NO: 150 - protein, 60 aa, synthetic construct
QIVLTQSPAIMSAFPGEKVTITCSATSSVNYMHWFQQKPGTSPKLWIYSSSNLASGVPAR

SEQ ID NO: 151 - protein, 60 aa, synthetic construct
QIVLTQSPAIMSASPGEKVTMTCSASSSVSYMHWYQQKSGTSPKTWIYDTSKLASGVPAR

SEQ ID NO: 152 - protein, 60 aa, synthetic construct
QIVLTQSPAIMSASPGEKVTISCSARSSVSYMYWYQQKSGSSPKPWIYRTSNLASGVPAR

SEQ ID NO: 153 - protein, 60 aa, synthetic construct
QIVLTQSPAIMSASPGEKVTITCSASSSVSYMHWFQQKPGTSPKLWIYSTSNLASGVPAR

SEQ ID NO: 154 - protein, 60 aa, synthetic construct
ELVMTQSPAIMSASPGEKVTMTCSASSSVNYMHWYQQKSGTSPKRWIYDTSKLASGVPAR

SEQ ID NO: 155 - protein, 59 aa, synthetic construct
LVLTQSPAIMSASPGEKVTMTCSASSSVSTVHWYQQKSGTSPKRWIYDTSKLASGVPAR

SEQ ID NO: 156 - protein, 241 aa, synthetic construct
QIVLTQSPTIMSASPGQKVTINCSAISSVNYMHWYQQKPGSSPKLWIYATSKLTLGVPARFSGSGSGTSYSLTVNSMVTEDATSYFCHQWSSYPPTFGAGTKLELKGSDSNAGHASAGNTSQVQLLQPGAELVRPGASVRLSCKTSGYTFTSYWINWVKQRPGQGLEWIGKIFPSDSHTNYNQKFKDKATLTVDKSSSTAYMQLISPTSEDSAVYYCTRDFDTQFYAMEYWGQGTSVTVSS

SEQ ID NO: 157 - protein, 241 aa, synthetic construct
QVQLLQPGAELVRPGASVRLSCKTSGYTFTSYWINWVKQRPGQGLEWIGKIFPSDSHTNYNQKFKDKATLTVDKSSSTAYMQLISPTSEDSAVYYCTRDFDTQFYAMEYWGQGTSVTVSSGSDSNAGHASAGNTSQIVLTQSPTIMSASPGQKVTINCSAISSVNYMHWYQQKPGSSPKLWIYATSKLTLGVPARFSGSGSGTSYSLTVNSMVTEDATSYFCHQWSSYPPTFGAGTKLELK

SEQ ID NO: 158 - DNA, 2549 bp, synthetic construct
  source 1..2549: mol_type = other DNA
atgaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcggagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatggggggggggggatgggggggggggggatgggggggggggggatgcaaattgtcctgacacaaagccctacaataatgagtgcatcaccgggacaaaaggttacaattaactgctctgcgattagtagcgtgaattgcatgcactggtatcagcagaagcccggttcttcacctaaactttggatatatgctactagtaagctgaccctcggagtaccggctttcctcgttttagtggaagtggatcagggaccagttacagcttgacggttaattcaatggttacagaagatgctacttcctatttttgccaccagtggtcttcttactacattcggggctggaactaaattggagttgaagggggggggggggatgggggggggggggatgggggggggggggatgcaggtgcagctgcttcagccaggggcagaacttgtacggcccggcgcaagcgtacgcttgtcctgcaaaacgagtggatatacgtttacctcctactggatcaactgggttaaacagagaccgggtcaaggtcttgagtggatcggtaagattttccccagtgattcccccactaactataatcaaaaatttaaggataaagctacgttgacggtcgataaatcatcttccaccgcgtatatgcaactgatatctccgaccagtgaggattctgccgtttactattgcacaagagatttcgacacgcagttctacgctatggaatactggggtcaggggacttctgtaacggtaagctctcgcagatag

SEQ ID NO: 159 - DNA, 2549 bp, synthetic construct
  source 1..2549: mol_type = other DNA
atgaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcggagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatggcagcgatagcaacgcgggccatgcgagcgcgggcaacaccagccaaattgtcctgacacaaagccctacaataatgagtgcatcaccgggacaaaaggttacaattaactgctctgcgattagtagcgtgaattacatgcactggtatcagcagaagcccggttcttcacctaaactttggatatatgctactagtaagctgaccctcggagtaccggctctcctcgttttagtggaagtggatcagggaccagttacagcttgacggttaattcaatggttacagaagatgctacttcctatttttgccaccagtggtcttcttactacattcggggctggaactaaattggagttgaagggcagcgatagcaacgcgggccatgcgagcgcgggcaacaccagccaggtgcagctgcttcagccaggggcagaacttgtacggcccggcgcaagcgtacgcttgtcctgcaaaacgagtggatatacgtttacctcctactggatcaactgggttaaacagagaccgggtcaaggtcttgagtggatcggtaagattttccccagtgattcccccactaactataatcaaaaatttaaggataaagctacgttgacggtcgataaatcatcttccaccgcgtatatgcaactgatatctccgaccagtgaggattctgccgtttactattgcacaagagatttcgacacgcagttctacgctatggaatactggggtcaggggacttctgtaacggtaagctctcgcagatag

SEQ ID NO: 160 - DNA, 2549 bp, synthetic construct
  source 1..2549: mol_type = other DNA
atgaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtcccgcactcttcactgtcccaattaaggaagcaggcggagactgccatgccccaacatacctacctgcggaggtggatggtgatgtcaaactcagttccaatctggtgattctacctggtcaagatctccaatatgttttggcaacctacgatacttcccaggttgaacatgctgtggtttattacgtttacagcccaagccgcttgagctcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatggcagcgatagcaacgcgggccatgcgagcgcgggcaacaccagccaggtgcagctgcttcagccaggggcagaacttgtacggcccggcgcaagcgtacgcttgtcctgcaaaacgagtggatatacgtttacctcctactggatcaactgggttaaacagagaccgggtcaaggtcttgagtggatcggtaagattttccccagtgattcccacactaactataatcaaaaatttaaggataaagctacgttgacggtcgataaatcatcttccaccgcgtatatgcaactgatatctccgaccagtgaggattctgccgtttactattgcacaagagatttcgacacgcagttctacgctatggaatactggggtcaggggacttctgtaacggtaagctctggcagcgatagcaacgcgggccatgcgagcgcgggcaacaccagccaaattgtcctgacacaaagccctacaataatgagtgcatcaccgggacaaaaggttacaattaactgctctgcgattagtagcgtgaattacatgcactggtatcagcagaagcccggttcttcacctaaactttggatatatgctactagtaagctgaccctcggagtaccggctcgttttagtggaagtggatcagggaccagttacagcttgacggttaattcaatggttacagaagatgctacttcctatttttgccaccagtggtcttcttatcctcctacattcggggctggaactaaattggagttgaagcgcagatag

SEQ ID NO: 161 - DNA, 1772 bp, synthetic construct
  source 1..1772: mol_type = other DNA
atgaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcggagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaat

SEQ ID NO: 162 - DNA, 1772 bp, synthetic construct
  source 1..1772: mol_type = other DNA
atgaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtcccgcactcttcactgtcccaattaaggaagcaggcggagactgccatgccccaacatacctacctgcggaggtggatggtgatgtcaaactcagttccaatctggtgattctacctggtcaagatctccaatatgttttggcaacctacgatacttcccaggttgaacatgctgtggtttattacgtttacagcccaagccgcttgagctcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaat

SEQ ID NO: 163 - DNA, 1670 bp, synthetic construct
  source 1..1670: mol_type = other DNA
agacttcatcgggcagccatctacaccgcagagatccataaaagcctcagcaccaatctagatgtaactactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtcccgcactcttcactgtcccaattaaggaagcaggcggagactgccatgccccaacatacctacctgcggaggtggatggtgatgtcaaactcagttccaatctggtgattctacctggtcaagatctccaatatgttttggcaacctacgatacttcccaggttgaacatgctgtggtttattacgtttacagcccaagccgcttgagctcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaat

SEQ ID NO: 164 - DNA, 45 bp, synthetic construct
  source 1..45: mol_type = other DNA
ggggggggggggatgggggggggggggatgggggggggggggatg

SEQ ID NO: 165 - DNA, 45 bp, synthetic construct
  source 1..45: mol_type = other DNA
ggcagcgatagcaacgcgggccatgcgagcgcgggcaacaccagc

SEQ ID NO: 166 - DNA, 39 bp, synthetic construct
  source 1..39: mol_type = other DNA
acaagagatttcgacacgcagttctacgctatggaatac

SEQ ID NO: 167 - DNA, 21 bp, synthetic construct
  source 1..21: mol_type = other DNA
gccatctcaagtgtaaattgc

SEQ ID NO: 168 - DNA, 21 bp, synthetic construct
  source 1..21: mol_type = other DNA
gccattagtagcgtgaattac

SEQ ID NO: 169 - DNA, 21 bp, synthetic construct
  source 1..21: mol_type = other DNA
gccattagtagcgtgaattgc

SEQ ID NO: 170 - DNA, 24 bp, synthetic construct
  source 1..24: mol_type = other DNA
ggatatacgtttacctcctactgg

SEQ ID NO: 171 - protein, 7 aa, synthetic construct
AISSVNY

SEQ ID NO: 172 - protein, 10 aa, synthetic construct
SAISSVNCHM

SEQ ID NO: 173 - protein, 10 aa, synthetic construct
SAISSVNYMH

SEQ ID NO: 174 - protein, 10 aa, synthetic construct
SAISSVNYHM

SEQ ID NO: 175 - protein, 241 aa, synthetic construct; identical to SEQ ID NO: 63

SEQ ID NO: 176 - protein, 119 aa, synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWSSNPPTFGGGTKLELKRADAAPTVSIFPP

SEQ ID NO: 177 - protein, 119 aa, synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWSNSPPTFGAGAKLELKRADAAPTVSIFPP

SEQ ID NO: 178 - protein, 120 aa, synthetic construct
GQIVLTQSPTIMSASPGEKVTMTCSASSSVDYMHWYQQKSGTSPKRWIYDTYKLASGVPARFSGSGSGTSYSLTINSMEAEDAATYYCQQWSSNPLTFGAGTKLELKRADAAPTVSIFPP

SEQ ID NO: 179 - protein, 110 aa, synthetic construct
QIVLTQSPAIMSAFPGEKVTITCSATSSVNYMHWFQQKPGTSPKLWIYSSSNLASGVPARFSGSGSGTSYSLTISRMEAEDAATYYCQQRSSYPITFGSGTKLEIKRADA

SEQ ID NO: 180 - protein, 119 aa, synthetic construct
DIVLTQSPAIMSASPGEKVTMTCSASSSVSYMHWYQQKSGTSPKRWIYDTSKLASGVPARFSGSGSGTSYSLTISSMEAEDAATYYCQQWSSNPPTFGAGTKLELKRTVAAPSVFIFPP

SEQ ID NO: 181 - DNA, 1772 bp, Measles morbillivirus; identical to SEQ ID NO: 161
  source 1..1772: mol_type = other DNA

SEQ ID NO: 182 - DNA, 1772 bp, synthetic construct
  source 1..1772: mol_type = other DNA
atgaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtcccgcactcttcactgtcccaattaaggaagcaggcggagactgccatgccccaacatacctacctgcggaggtggatggtgatgtcaaactcagttccaatctggtgattctacctggtcaagatctccaatatgttttggcaacctacgatacttccgcagttgaacatgctgtggtttattacgtttacagcccaagccttgagctttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaat

SEQ ID NO: 183 - DNA, 2549 bp, synthetic construct
  source 1..2549: mol_type = other DNA
atgaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcggagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatggggggggggggagtggggggggggggagtggggggggggggagtcaggtgcagctgcttcagccaggggcagaacttgtacggcccggcgcaagcgtacgcttgtcctgcaaaacgagtggatatacgtttacctcctactggatcaactgggttaaacagagaccgggtcaaggtcttgagtggatcggtaagattttccccagtgattcccacactaactataatcaaaaatttaaggataaagctacgttgacggtcgataaatcatcttccaccgcgtatatgcaactgatatctccgaccagtgaggattctgccgtttactattgcacaagagatttcgacacgcagttctacgctatggaatactggggtcaggggacttctgtaacggtaagctctggggggggggggagtggggggggggggagtggggggggggggagtcaaattgtcctgacacaaagccctacaataatgagtgcatcaccgggacaaaaggttacaattaactgctctgcgattagtagcgtgaattacatgcactggtatcagcagaagcccggttcttcacctaaactttggatatatgctactagtaagctgaccctcggagtaccggctcgttttagtggaagtggatcagggaccagttacagcttgacggttaattcaatggttacagaagatgctacttcctatttttgccaccagtggtcttcttatcctcctacattcggggctggaactaaattggagttgaagcgcagatag

SEQ ID NO: 184 - DNA, 2549 bp, synthetic construct
  source 1..2549: mol_type = other DNA
atgaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtcccgcactcttcactgtcccaattaaggaagcaggcggagactgccatgccccaacatacctacctgcggaggtggatggtgatgtcaaactcagttccaatctggtgattctacctggtcaagatctccaatatgttttggcaacctacgatacttccgcagttgaacatgctgtggtttattacgtttacagcccaagccttgagctttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatggggggggggggagtggggggggggggagtggggggggggggagtcaggtgcagctgcttcagccaggggcagaacttgtacggcccggcgcaagcgtacgcttgtcctgcaaaacgagtggatatacgtttacctcctactggatcaactgggttaaacagagaccgggtcaaggtcttgagtggatcggtaagattttccccagtgattcccacactaactataatcaaaaatttaaggataaagctacgttgacggtcgataaatcatcttccaccgcgtatatgcaactgatatctccgaccagtgaggattctgccgtttactattgcacaagagatttcgacacgcagttctacgctatggaatactggggtcaggggacttctgtaacggtaagctctggggggggggggagtggggggggggggagtggggggggggggagtcaaattgtcctgacacaaagccctacaataatgagtgcatcaccgggacaaaaggttacaattaactgctctgcgattagtagcgtgaattacatgcactggtatcagcagaagcccggttcttcacctaaactttggatatatgctactagtaagctgaccctcggagtaccggctcgttttagtggaagtggatcagggaccagttacagcttgacggttaattcaatggttacagaagatgctacttcctatttttgccaccagtggtcttcttatcctcctacattcggggctggaactaaattggagttgaagcgcagatag

SEQ ID NO: 185 - DNA, 1536 bp, synthetic construct
  source 1..1536: mol_type = other DNA
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgccccagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaagccgaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 186 - DNA, 1010 bp, synthetic construct
  source 1..1010: mol_type = other DNA
gaattcatgcaggtgcagctgcttcagccaggggcagaacttgtacggcccggcgcaagcgtacgcttgtcctgcaaaacgagtggatatacgtttacctcctactggatcaactgggttaaacagagaccgggtcaaggtcttgagtggatcggtaagattttccccagtgattcccacactaactataatcaaaaatttaaggataaagctacgttgacggtcgataaatcatcttccaccgcgtatatgcaactgatatctccgaccagtgaggattctgccgtttactattgcacaagagatttcgacacgcagttctacgctatggaatactggggtcaggggacttctgtaacggtaagctctggcagcgatagcaacgcgggccatgcgagcgcgggcaacaccagccaaattgtcctgacacaaagccctacaataatgagtgcatcaccgggacaaaaggttacaattaactgctctgcgattagtagcgtgaattacatgcactggtatcagcagaagcccggttcttcacctaaactttggatatatgctactagtaagctgaccctcggagtaccggctcgttttagtggaagtggatcagggaccagttacagcttgacggttaattcaatggttacagaagatgctacttcctatttttgccaccagtggtcttcttatcctcctacattcggggctggaactaaattggagttgaagtgagaattcactacaactccagcacccagaccccctacacctgctccaactatcgcaagtcagcccctgtcactgcgccctgaagcctgtattgtcatcatcaccgtggtggcggctgccgtgatcatgggcaccgccggtctctccacttatctgtacaaccgccagcgtaaaatcaagaagtaccgcctgtagctcaaatcctgcacaacagattcttcatgtttggaccaaatcaacttgtgataccatgctcaaagaggcctcaattatatttg

SEQ ID NO: 187 - DNA, 21 bp, synthetic construct
  source 1..21: mol_type = other DNA
ttcagtcaccatgctgttgac

SEQ ID NO: 188 - DNA, 18 bp, synthetic construct
  source 1..18: mol_type = other DNA
ccagcagaagccaggatc

SEQ ID NO: 189 - protein, 377 aa, synthetic construct
METDTLLLWVLLLWVPGSTGQKVTSLTACLVDQSLRLDCRHENTSSSPIQYEFSLTRETKKHVLFGTVGVPEHTYRSRTNFTSKYNMKVLYLSAFTSKDEGTYTCALHHSGHSPPISSQNVTVLRDKLVKCEGGSGGGSGGGVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

(19) United States
(12) Patent Application Publication
(10) Pub. No.: US 2025/0249125 A1
(43) Pub. Date: Aug. 7, 2025

(54) ANTI-CD90 ANTIBODIES, BINDING FRAGMENTS, AND USES THEREOF

(71) Applicant: Fred Hutchison Cancer Center, Seattle, WA (US)

(72) Inventors: Hans-Peter Kiem, Seattle, WA (US); Stefan Radtke, Seattle, WA (US); Kurt Berckmueller, Seattle, WA (US); Roland K. Strong, Seattle, WA (US); Peter Rupert, Seattle, WA (US)

(73) Assignee: Fred Hutchison Cancer Center, Seattle, WA (US)

(21) Appl. No.: 18/855,248

(22) PCT Filed: Mar. 22, 2023
(86) PCT No.: PCT/US2023/064830

§ 371 Date: Oct. 8, 2024

Related U.S. Application Data
(60) Provisional application No. 63/329,258, filed on Apr. 8, 2022.

(51) Int. Cl. A61K 48/00; A61K 47/68; A61K 49/00; A61K 51/10; A61K 51/12; C07K 14/005; C07K 16/28; C12N 9/22; C12N 15/11; C12N 15/86

(52) U.S. Cl. CPC A61K 48/005; A61K 47/6849; A61K 49/0043; A61K 49/0045; A61K 49/0058; A61K 51/1033; A61K 51/1244; C07K 14/005; C07K 16/2803; C07K 16/2809; C07K 16/2815; C07K 16/2818; C07K 16/2878; C12N 9/222; C12N 15/11; C12N 15/86; C07K 2317/24; C07K 2317/31; C07K 2317/565; C07K 2317/622; C12N 2310/20; C12N 2740/10043; C12N 2740/10045; C12N 2740/15043; C12N 2740/15045; C12N 2750/14143; C12N 2750/14145; C12N 2760/18422; C12N 2800/22

(57) ABSTRACT

Anti-CD90 antibodies, binding fragments, and uses thereof are described. The provided antibodies and binding fragments can be used to isolate CD90 cells or to target such cells ex vivo or in vivo for a research, a diagnostic, or a therapeutic purpose. Anti-CD90 antibodies and binding fragments thereof can also be used in engineered formats to create antibody conjugates or within a recombinant protein to pseudotype viral vectors.

### CROSS-REFERENCE TO RELATED APPLICATIONS

This application is a U.S. National Phase Patent Application based on International Patent Application No. PCT/US2023/064830 filed Mar. 22, 2023, which claims priority to U.S. Provisional Patent Application No. 63/329,258 filed Apr. 8, 2022, both of which are incorporated herein by reference in its entirety as if fully set forth herein.

### STATEMENT REGARDING FEDERALLY SPONSORED RESEARCH OR DEVELOPMENT

This invention was made with government support under A1135953 awarded by the National Institutes of Health. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is 3AJ3524.XML. The file is 196,608 bytes, was created on Aug. 30, 2024, and is being submitted electronically via Patent Center.

### FIELD OF THE DISCLOSURE

The present disclosure provides anti-CD90 antibodies, binding fragments, and uses thereof. The provided antibodies and binding fragments can be used to isolate CD90 cells or to target such cells ex vivo or in vivo for a research, diagnostic, or therapeutic purposes.

### BACKGROUND OF THE DISCLOSURE

CD90, also referred to as Thy-1, is a 25-37 KDa glycosylphosphatidylinositol (GPI)-anchored glycoprotein. CD90 is expressed on hematopoietic stem cells (HSC) and mesenchymal stem/stromal cells (MSC), two therapeutically important cell types. For example, HSC are the basis for therapeutic cord blood, bone marrow and mobilized peripheral blood stem cell transplantation approaches, and blood stem cell gene therapy approaches. HSC transplantation can be curative in many non-malignant and malignant disorders including severe combined immune deficiency (SCID), Fanconi anemia (FA), and several blood cancers. MSC are adult, multipotent non-hematopoietic stem cells. MSC have demonstrated therapeutic effects in several diseases such as in liver diseases, kidney diseases, cardiovascular diseases, neurological diseases, and immune diseases.

### SUMMARY OF THE DISCLOSURE

The current disclosure provides antibodies and binding fragments thereof that bind CD90. Among other uses, the antibodies and binding fragments thereof can be used to target hematopoietic stem cells (HSC) and mesenchymal stem/stromal cells (MSC) in vivo or ex vivo for research, diagnostic, and therapeutic purposes.

In certain examples, the disclosed antibodies and binding fragments thereof are used to create single chain variable fragments (scFv). In other examples, the disclosed antibodies and binding fragments thereof can be used to enhance the cell specificity of viral vector-based gene delivery. For example, a binding fragment of the disclosed antibodies can be used to create a recombinant protein with a viral structural protein (e.g., the measles virus hemagglutinin protein or vesicular stomatitis virus G protein). This recombinant protein can be used to pseudotype other viral vectors, such as lentiviral vectors.

The antibodies and binding fragments thereof can also be engineered into numerous additional formats, such as antibody-immunotoxin conjugates, antibody-drug conjugates (ADCs), antibody-detectable label conjugates, antibody-radioisotope conjugates, antibody-nanoparticle conjugates, antibody-bead conjugates, and antibodies with multiple binding domains.

### BRIEF DESCRIPTION OF THE SEVERAL VIEWS OF THE DRAWINGS

Some of the drawings submitted herein may be better understood in color. Applicant considers the color versions of the drawings as part of the original submission and reserves the right to present color images of the drawings in later proceedings.

FIG. 1. CD90 5E10 heavy and light chain sequences. Sequence based on Sanger Sequencing of PCR-amplified chain coding regions with complementarity determining region (CDR) encoding segments (according to IMGT CDR Definition) underlined. Sequences include naïve CD90 5E10 heavy and light chain encoding sequences; codon-optimized CD90 5E10 heavy and light chain encoding sequences; codon-optimized CD90 5E10 heavy and light chain amino acid sequences; and codon-optimized CD90 5E10 with mutated unpaired cysteine residues for the light chain encoding sequence and associated protein sequence. Dunbar et al. Nucleic Acids Res. 2016; 44:w474-w478.

FIGS. 2A, 2B. Naïve and codon optimized CD90 5E10 heavy chain (2A) and light chain (2B) encoding sequence alignments. Substituted base pairs are indicated, whereas homologous base pairs are indicated by a dot. Codon optimization performed with assistance from the Codon Usage Database and the GenScript® (GenScript Biotech Co., Piscataway, NJ) Codon Usage Frequency Table Tool.

FIG. 3. Identification of non-conserved cysteine residues in the light chain. Sequences were compared to the data repository to identify potential residues that cause problems during the folding and production of scFvs. Two cysteines at position 92 and 178 were identified that are predicted to not form di-sulfate bonds. Both cysteines were mutated into tyrosine and arginine as found in the other 5 reference sequences (see details in FIGS. 1 and 7).

FIGS. 4A-4C. (4A) CD90 scFv design KB1. Engineered codon optimized anti-CD90 scFv sequence (SEQ ID NO: 63) connecting the light chain to the heavy chain with the GGGGSGGGGSGGGGS (SEQ ID NO: 64) linker sequence. (4B) CD90 scFv design KB2. Engineered codon optimized with mutated cysteines anti-CD90 scFv sequence (SEQ ID NO: 156) connecting the light chain to the heavy chain with the GSDSNAGHASAGNTS (SEQ ID NO: 66) linker sequence. (4C) CD90 scFv design KB3. Engineered codon optimized with mutated cysteines anti-CD90 scFv sequence (SEQ ID NO: 157) connecting the heavy chain to the light chain with the GSDSNAGHASAGNTS (SEQ ID NO: 66) linker sequence. Light chain segments are italicized, CDRs are underlined, linker is in double underlined, and mutated unpaired cysteines are in bold.

FIGS. 5A-5C. Measles Hemagglutinin CD90 scFv Design. Engineered sequence of the CD90-targeted Measles-pseudotyped lentiviral envelope plasmid. Anti-CD90 scFvs shown in FIGS. 4A-4C are attached to the Hemagglutinin Endodomain connected through a linker sequence. Plasmids containing Measles Hemagglutinin provided by EIs Verhoeyen at the Universite de Lyon (Frecha, et al., Blood 2008; 112 (13): 4843-4852). (5A) Measles Hemagglutinin KB1 scFv Design (SEQ ID NO: 158). (5B) Measles Hemagglutinin KB2 scFv Design (SEQ ID NO: 159). (5C) Measles Hemagglutinin KB3 scFv Design (SEQ ID NO: 160). Hemagglutinin ectodomain is in normal font (residues 1-102), hemagglutinin endodomain is underlined (residues 103-1772), GGGGS linkers are in bold, CD90 scFv variable light chain is in italics, CD90 scFv variable heavy chain is double underlined, the tail is in bold italics (residues 2541-2549), removed cysteine residues are in boxes, and knockout regions are in bold and underlined.

FIG. 6. Measles Production Schematic. For the production of Measles-pseudotyped lentiviral vectors (LV), HEK-293T cells are transfected with four plasmids, pRSCP newGFPwpre GFP plasmid(V), pCMVdelta8.74 Gag/POL(X), HΔ24(Y) and FΔ30(Z) in the ratio V(19.1):X(14.5):Y(7.5):Z(7.5). Transfected producer cells are cultured for 40 hours, supernatant harvested, and concentrated by centrifugation (6000G, 24 Hrs). Viral production protocol is provided by EIs Verhoeyen at the Universite de Lyon (Frecha, et al., Blood 2008; 112 (13): 4843-4852). Vectors 115 pRSCP GFP and Plasmid #22 Gag/Pol are described in Trobridge, et al., Blood. 2008; 111(12):5537-43).

FIGS. 7A, 7B. CD90 scFv Thy-1 Docking Modeling. Computer generated structural prediction and interaction of the designed anti-CD90 scFv with CD90. Model generated in SABPred (Dunbar, et al., Nucleic Acids Res. 2016; 44. W474-W478), docking estimated with Zdock (Pierce, et al., Bioinformatics 2014; 30(12): 1771-3), and visualized in Pymol (The PyMOL Molecular Graphics System, Version 2.0 Schrödinger, LLC.) (7A) Codon-optimized CD90 scFv. (7B) Codon-optimized CD90 with mutated cysteines. VL is in the left bottom, VH is in the right bottom, CDR regions are in the center between the VL and VH, Thy-1/CD90 protein is at the top. Mutated cysteines are indicated.

FIGS. 8A, 8B. CD90ex-mCherry production for scFv testing. In order to test the functionality of CD90 scFvs and ability to bind CD90 protein, a custom fusion protein merging the extracellular domain of CD90 (CD90ex) with the fluorochrome mCherry (CD90ex-mCh) was created. Interaction of this fusion protein with anti-CD90 Fab2 fragments was confirmed by size exclusion chromatography (SEC, 8A) and SDS PAGE of the eluted peaks (8B). CD90ex-mCh successfully interacted with the anti-CD90 Fab2 fragments creating a complex (UV A200 cd90-mCherry-5E10-FAb) that showed increased size and was eluted in a fraction distinct from CD90ex-mCh (UV A200 cd90-mCherry) and CD90 Fab2s (UV A200 5E10 Fab) alone. (7B) Lanes of SDS PAGE are: Lane 1 is Ez-prot-stan; Lane 2 is isolated 5E10 Fab (5E10 Fab treatment, 15.5 ml peak); Lane 3 is isolated CD90-mcherry (CD90-mCherry treatment. 14.3 ml peak); Lane 4 is complex peak 1 (cd90-mCherry-5E10-Fab treatment, 12.7 ml peak); and Lane 5 is complex peak 2 (cd90-mCherry-5E10-Fab treatment, 14.3 ml peak).

FIGS. 9A, 9B. Binding of CD90ex-mCherry to CD90-targeted viruses. (9A) Schematic showing quality control of Measles virus. CD90-targeted viruses were incubated with CD90ex-mCh and the interaction measured with a Zetaviewer measuring the size of particles. (9B) Untargeted measles-pseudotyped lentiviral viruses were 100-140 nm in size (panel 1 and panel 2; upper far left and lower far left graphs) and did not show a size increase when incubated with CD90ex-mCH (panel 2; lower far left graph). Virus design KB1 (without mutated cysteines) was similarly sized and did now show a size increase when incubated with CD90ex-mCh (panel 3 and panel 4; second from the left, upper and lower graphs). Virus design KB2 (panel 5 and panel 6; second from the right, upper and lower graphs) and KB3 (panel 7 and panel 8; upper far right and lower far right graphs) (mutated cysteines, VL-VH, VH-VL) both demonstrated interaction with CD90ex-mCh and the appearance of larger particles of 190-200 nm in size (panel 6 and panel 8; lower second from right and lower far right graphs, arrows) indicating interaction of the CD90 scFvs with CD90 protein.

FIG. 10. Additional sequences supporting the disclosure. Primers used in sequencing CD90 Ab Variant Heavy and Variant Light Chains (von Boehmer et al., Nat Protoc. 2016 October; 11(10):1908-1923): forward primer for sequencing CD90 VL Ab Variant (SEQ ID NO: 70), reverse primer for sequencing CD90 VL Ab Variant (SEQ ID NO: 71), forward primer for sequencing CD90 VH Ab Variant (SEQ ID NO: 72), reverse primer for sequencing CD90 VH Ab Variant (SEQ ID NO: 73). Additional sequences include hemagglutinin (SEQ ID NO: 74), engineered CD90-targeted Measles-pseudotyped lentiviral envelope plasmid (SEQ ID NO: 161), engineered CD90-targeted Measles-pseudotyped Lentiviral Envelope with knockout regions (SEQ ID NO: 162), engineered CD90-targeted Measles-pseudotyped lentiviral envelope plasmid ectodomain (SEQ ID NO: 76), engineered CD90-targeted Measles-pseudotyped lentiviral envelope plasmid endodomain (SEQ ID NO: 77), engineered CD90-targeted Measles-pseudotyped lentiviral envelope plasmid endodomain with knockout regions (SEQ ID NO:163), linkers (SEQ ID NOs: 78, 164, and 165), and tail of engineered CD90-targeted Measles-pseudotyped lentiviral envelope plasmid.

FIGS. 11A-11E. Design and computational validation of an anti-CD90 scFv. (11A) Workflow to determine the variant heavy (VH) and variant light (VL) sequence from the hybridoma cell line producing the CD90 antibody clone 5E10. (11B) Amino acid sequence of the initial scFv design connecting the VL and VH chain with a 3x(GGGGS) (SEQ ID NO: 64) linker (italicized and bolded) sequence. Complementarity-determining regions (CDRs, color coded) for the VH and VL sequence were determined using IMGT. (11C) Predicted interaction of CD90 with the anti-CD90 scFv. CDRs of the scFv are shown. (11D) Representative database crystal structure highlighting the regions of putative mismatched cysteines from our anti-CD90 scFv. (11E) Sequence alignment of our anti-CD90 scFv with the reference database for highly homologous crystal structures. Mismatched cysteines in position 31 and 59 are shown enclosed in boxes.

FIGS. 12A-12J: Design and quality control of CD90-targeted measles-pseudotyped lentiviral vectors. (12A) Variant H1 WT measles. (12B) Summary of designed Measles envelopes and the expected antigen recognition. (12C) Variant H2 KO measles annotated sequence. (12D) Variant H3 WT measles and targeting domain annotated sequence. (12E) Variant H4 KO measles and targeting domain annotated sequence. (12F) Viral variants were tested on HT1080 cells demonstrating inability of the KO versions to fuse and transduce. (12G) Workflow for the hydrodynamic titration of engineered viral vectors using nanoparticle tracking analysis (NTA). (12H) Concentration of viral particles per ml culture medium harvested. (12I) Concentration of RNA-loaded viral particles per ml culture medium measured with SYBR II. (12J) RNA loading efficiency calculated based on values from FIGS. 12H and 12I.

FIGS. 13A-13C: Assessment of antigen recognition by CD90-targeted lentiviral vectors. (13A) Quality control of custom produced CD90-mCherry fusion protein. CD90 Fab (peak 2), CD90-mCherry fusionprotein (peak 3), and a mix of CD90 Fab with CD90-mCherry were analyzed by size exclusion chromatograph (line graph). Fractions/peaks indicated with numbers were collected and content validated by SDA PAGE (lower panel). (13B) Workflow for the assessment of CD90-mCherry interaction with CD90-targeted viral vectors using nanoparticle tracking analysis (NTA). (13C) Size of viral particles measuring scattered light (scatter, top row). Size distribution of mRNA-loaded particles stained with SYBR II and measuring the fluorescence in the 500 nm filter (+SYBR II, middle row). SYBR II signal of mRNA-loaded viral particles co-incubated with CD90-mCh measuring the fluorescence in the 500 nm filter (+SYBR II+CD90-mCh, bottom row). Numbers in each plot indicate the average size of numbered peaks.

FIGS. 14A-14C: Selective transduction of cell lines and HSCs with CD90-targeted viral vectors. (14A) Flow-cytometric assessment of the transduction efficiency of Jurkat cells using all 4 vector variants. Efficiencies are visualized relative to the efficiency of variant H1 (mean±SD). (14B) Correlation of the number of transduced cells with the number of mRNA-loaded viral particles using the WT variant H1. R²: Spearman's rank correlation coefficient. (14C) Functional assessment of transduced human CD34+ cells in colony-forming cell (CFC) assays. mScarlet expressing CD34+ cells were FACS-purified and introduced into primary (1^{st}) CFC assays (upper left graph). After 14 days, colonies were counted, all cells harvested, and 5% of all cells replated into secondary (2^{nd}) CFC assays (upper right graph). Secondary CFCs were counted after 14 days, all cells harvested, the total number of WBCs counted (lower left graph), and the mScarlet expression measured by flow-cytometry (lower right graph).

FIGS. 15A-15E: Design and quality control of CD90-targeted VSV-G-pseudotyped lentiviral vectors. (15A) VSV-G variant V2 and V4 binder KO sequence. (15B) VSV-G variant V3 and V4 CD90 scFv annotated sequence. (15C) Summary of designed VSV-G envelopes and the expected antigen recognition. (15D) Concentration of total and RNA-loaded viral particles per ml culture medium measured with NTA (top two graphs). Calculated RNA loading efficiency (bottom graph). (15E) Transduction of 200.000 K562 cells with all four VSV-G variants at different doses as indicated below the graph. Transduction efficiency (mScarlet expression) measured by flow-cytometry.

FIGS. 16A-16C: On-target specificity of CD90-targeted vectors. (16A) Flow-cytometric assessment of CD90 expression on Raji and Jurkat cells. (16B) Transduction efficiency of mixed cultures containing GFP⁻ Raji and GFP⁺ Jurkat cells with either Variant V1 or V4. (16C) On target specificity of Measles- and VSV-G-based vector variants 1 and 4 determined by the ratio of mScarlet⁺GFP⁻ cells within total GFP⁻ Raji to mScarlet⁺GFP⁺ cells within total GFP⁺ Jurkat cells.

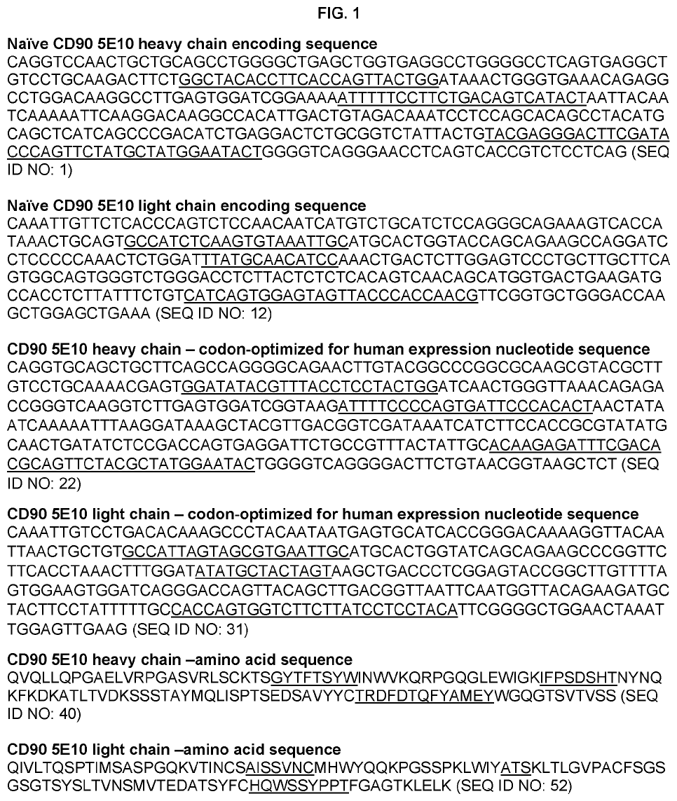

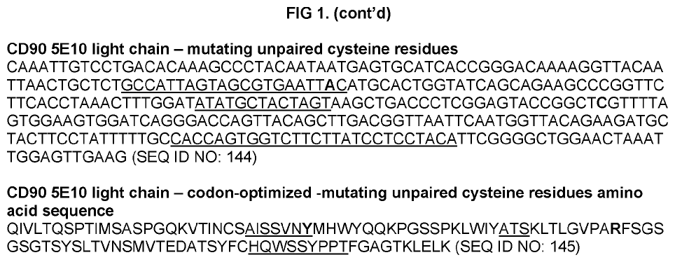

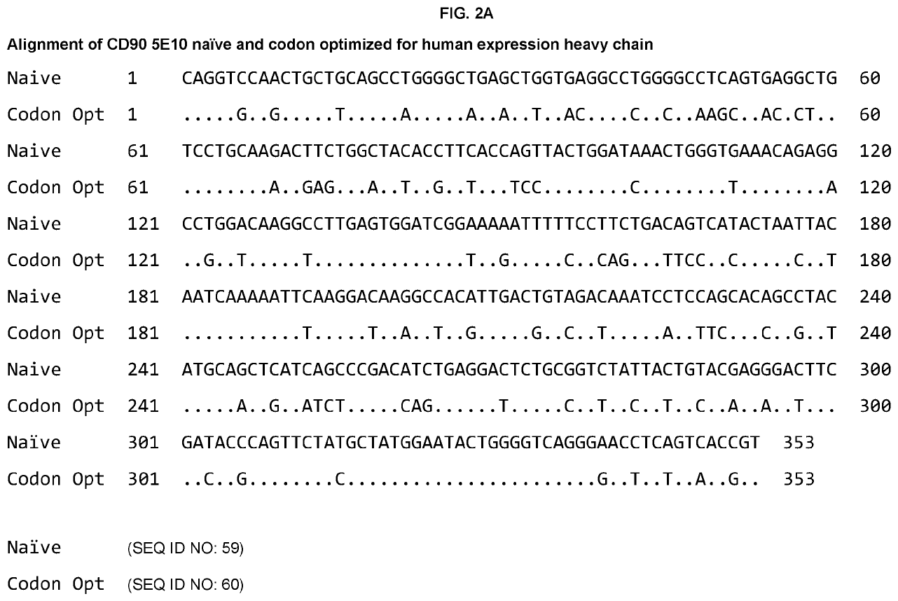

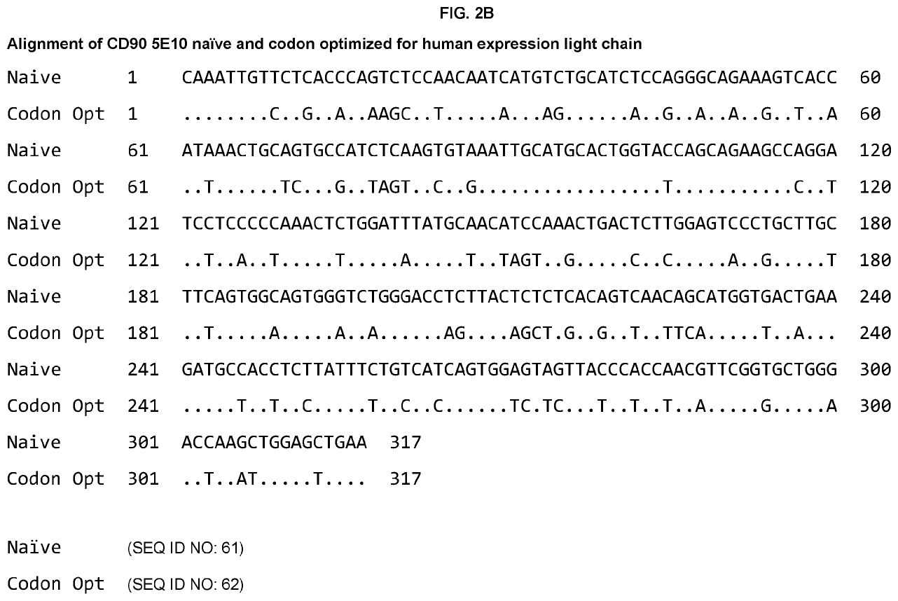

### DETAILED DESCRIPTION

CD90, also referred to as Thy-1, is a 25-37 KDa glycosylphosphatidylinositol (GPI)-anchored glycoprotein. CD90 is expressed on hematopoietic stem cells (HSC) and mesenchymal stem/stromal cells (MSC), two therapeutically important cell types. For example, HSC are the basis for therapeutic cord blood, bone marrow and mobilized peripheral blood stem cell transplantation approaches, and blood stem cell gene therapy approaches. HSC transplantation or modification can be curative in immune deficiencies. More than 80 primary immune deficiency diseases are recognized by the World Health Organization. These diseases are characterized by an intrinsic defect in the immune system in which, in some cases, the body is unable to produce any or enough antibodies against infection. In other cases, cellular defenses to fight infection fail to work properly. Typically, primary immune deficiencies are inherited disorders.

Secondary, or acquired, immune deficiencies are not the result of inherited genetic abnormalities, but rather occur in individuals in which the immune system is compromised by factors outside the immune system. Examples include trauma, viruses, chemotherapy, toxins, and pollution. Acquired immunodeficiency syndrome (AIDS) is an example of a secondary immune deficiency disorder caused by a virus, the human immunodeficiency virus (HIV), in which a depletion of T lymphocytes renders the body unable to fight infection.

X-linked severe combined immunodeficiency (SCID-X1) is both a cellular and humoral immune depletion caused by mutations in the common gamma chain gene (γC), which result in the absence of T and natural killer (NK) lymphocytes and the presence of nonfunctional B lymphocytes. SCID-X1 is fatal in the first two years of life unless the immune system is reconstituted, for example, through bone marrow transplant (BMT) or gene therapy.

Fanconi anemia (FΔ) is an inherited blood disorder that leads to bone marrow failure. It is characterized, in part, by a deficient DNA-repair mechanism. At least 20% of patients with FA develop cancers such as acute myeloid leukemias, and cancers of the skin, liver, gastrointestinal tract, and gynecological systems. The skin and gastrointestinal tumors are usually squamous cell carcinomas. The average age of patients who develop cancer is 15 years for leukemia, 16 years for liver tumors, and 23 years for other tumors.

Cells from FA patients display a characteristic hypersensitivity to agents that produce interstrand DNA crosslinks such as mitomycin C or diepoxybutane. FA genes define a multicomponent pathway involved in cellular responses to DNA cross-links. Five of the FA genes (FANCA, FANCC, FANCE, FANCF and FANCG) have been cloned and the FANCA, FANCC and FANCG proteins have been shown to form a molecular complex with primarily nuclear localization. A number of mutations in the FANCC gene have been identified which are correlated with FA of differing degrees of severity.

Mesenchymal stem/stromal cells (MSC) are adult, multipotent, non-hematopoietic stem cells. MSC have demonstrated therapeutic effects in several diseases and their effects largely mediated by paracrine factors such as exosomes. Exosomes are 40-150 nm membrane-bound vesicles generated in multivesicular bodies and secreted when these compartments fuse with the plasma membrane. MSC-derived exosomes function in cell-cell communication as they can contain cytokines and growth factors, such as lipids, proteins, mRNAs, transfer RNA (tRNA), long noncoding RNAs (lncRNAs), microRNAs (miRNAs) and mitochondrial DNA (mtDNA) and once released into the extracellular space, they can be taken up by other cells or carried to distant sites via biological fluid.

Most exosomes include similar proteins such as tetraspanins (CD81, CD63, and CD9), heat-shock proteins (HSP60, HSP70 and HSP90), ALIX and tumor susceptibility gene 101 (TSG101); and have tissue type-specific proteins according to their cell source. MSC-derived exosomes have been used to treat, for example,
liver diseases (Li T, et al., Stem Cells Dev. 2013; 22(6):845-54; Tan, et al. Stem Cell Res Ther. 2014; 5(3):76; Fouraschen, et al., Stem Cells Dev. 2012; 21(13):2410-9; and Lou, et al., Exp Mol Med. 2017; 49(6):e346),
kidney diseases (Zou, et al., Am J Transl Res. 2016; 8(10):4289-99; Eirin A, et al., Kidney Int. 2017; 92(1):114-24; Bruno, et al., J Am Soc Nephrol. 2009; 20(5):1053-67; Bruno, et al., PLoS One. 2012; 7(3):e33115; Zhang, et al., Kidney Blood Press Res. 2016; 41(2):119-28; and Tomasoni, et al., Stem Cells Dev. 2013; 22(5):772-80),
cardiovascular diseases (Suzuki, et al., Adv Exp Med Biol. 2017; 998:179-85; Cui, et al., J Cardiovasc Pharmacol. 2017; 70(4):225-31; Wang, et al., Stem Cells Transl Med. 2017; 6(1):209-22; Arslan, et al., Stem Cell Res. 2013; 10(3):301-12; Feng, et al., PLoS One. 2014; 9(2):e88685; Xiao, et al., Circ Res. 2018; 123(5):564-78; Mayourian, et al., Circ Res. 2018; 122(7):933-44; and Liu, et al., Cell Physiol Biochem. 2017; 43(1):52-68),
neurological diseases (Luarte, et al., Stem Cells Int. 2016; 2016:5736059; Cooper, et al., Mov Disord. 2014; 29(12):1476-85; Xin, et al., Stroke. 2017; 48(3):747-53; Katsuda, et al., Sci Rep. 2013; 3:1197; Yang, et al., J Mol Neurosci. 2018; 65(1):74-83; and Cui, et al., FASEB J. 2018; 32(2):654-68),
and immune diseases (You, et al., J Hematol Oncol. 2017; 10(1):165; Lai, et al., J Hematol Oncol. 2018; 11(1):135; Shigemoto-Kuroda, et al., Stem Cell Reports. 2017; 8(5):1214-25; Du, et al., Exp Cell Res. 2018; 363(1):114-20; and Li, et al., EBioMedicine. 2016; 8:72-82).

The current disclosure provides antibodies and binding fragments thereof that bind CD90. Among other uses, the antibodies and binding fragments thereof can be used to target HSC and MSC in vivo or ex vivo for research, diagnostic, and/or therapeutic purposes.

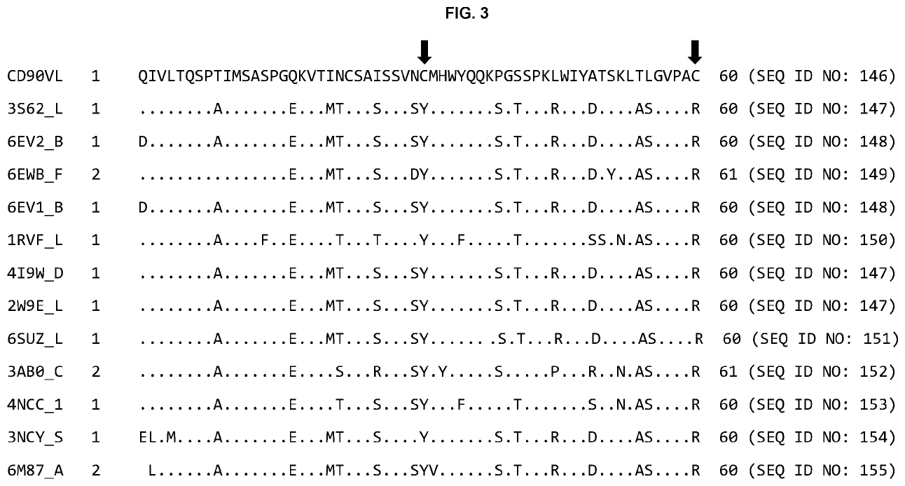

Naïve anti-CD90 antibody sequences were codon-optimized to provide the codon-optimized anti-CD90 antibody sequences and encoding sequences provided herein. The amino acid codon-optimized sequences were aligned to other antibody sequences (see FIG. 3) and cysteine residues were found within the codon-optimized variable light chain that did not align with the other antibody sequences. These two cysteine residues were mutated to match the residues in the antibodies used in the alignment to produce the codon-optimized anti-CD90 antibody light chain with mutated cysteines.

In certain examples, the disclosed antibodies and binding fragments thereof are used to create single chain variable fragments (scFv). In other examples, the disclosed antibodies and binding fragments thereof can be used to enhance the cell specificity of viral vector-based gene delivery. For example, a binding fragment of the disclosed antibodies can be used to create a recombinant protein with a viral structural protein (e.g., the measles virus hemagglutinin protein or the vesicular stomatitis virus G (VSV-G) protein). This recombinant protein can be used to pseudotype other viral vectors, such as lentiviral vectors.

The antibodies and binding fragments thereof can also be engineered into numerous additional formats, such as antibody-immunotoxin conjugates, antibody-drug conjugates (ADCs), antibody-detectable label conjugates, antibody-radioisotope conjugates, antibody-nanoparticle conjugates, antibody-bead conjugates, and antibodies with multiple binding domains.

Having highlighted key aspects of the current disclosure, the following additional details and options to practice the disclosure are provided as follows: (i) CD90 Antibodies & Binding Fragments Thereof; (ii) Use of CD90 Antibodies & Binding Fragments Thereof to Enhance Cell Specificity of Viral Vector Delivery; (iii) Anti-CD90 Antibody Conjugates (including antibody-immunotoxin conjugates, antibody-drug conjugates (ADCs), antibody-detectable label conjugates, antibody-radioisotope conjugates, antibody-nanoparticle conjugates, antibody-bead conjugates, and multiple binding domain antibodies); (iv) Compositions for Administration; (v) Ex Vivo Cell Manufacturing & Formulation; (vi) In Vivo or Ex Vivo Genetic Modification of CD90+ Cells; (vii) Methods of Use; (viii) Exemplary Embodiments; (ix) Experimental Example; and (x) Closing Paragraphs. These headings are provided for organization purposes only and should not be construed to limit the teachings or interpretation of the current disclosure.

### (i) CD90 Antibodies & Binding Fragments Thereof

The current disclosure provides anti-CD90 antibodies and binding fragments thereof. Naturally occurring antibody structural units include a tetramer. Each tetramer includes two pairs of polypeptide chains, each pair having one light chain and one heavy chain. The amino-terminal portion of each chain includes a variable region that is responsible for antigen recognition and epitope binding. The variable regions exhibit the same general structure of relatively conserved framework regions (FR) joined by three hyper variable regions, also called complementarity determining regions (CDRs). The CDRs from the two chains of each pair are aligned by the framework regions, which enables binding to a specific epitope. From N-terminal to C-terminal, both light and heavy chain variable regions include the domains FR1, CDR1, FR2, CDR2, FR3, CDR3 and FR4. The assignment of amino acids to each domain is typically in accordance with the definitions of IMGT (Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme)), Kabat numbering (Kabat et al. (1991), “Sequences of Proteins of Immunological Interest,” 5th Ed. Public Health Service, National Institutes of Health, Bethesda, Md. (“Kabat” numbering scheme)); Chothia (Al-Lazikani et al., (1997) JMB 273, 927-948 (“Chothia” numbering scheme)), Martin (Abinandan et al., Mol Immunol. 45:3832-3839 (2008), “Analysis and improvements to Kabat and structurally correct numbering of antibody variable domains”), Gelfand, Contact (MacCallum et al., J. Mol. Biol. 262:732-745 (1996), “Antibody-antigen interactions: Contact analysis and binding site topography,” J. Mol. Biol. 262, 732-745.” (Contact numbering scheme)), AHo (Honegger A and Plückthun A, “Yet another numbering scheme for immunoglobulin variable domains: an automatic modeling and analysis tool,” J Mol Biol, 2001 Jun. 8; 309(3):657-70, (AHo numbering scheme)), North (North et al., J Mol Biol. 406(2):228-256 (2011), “A new clustering of antibody CDR loop conformations”), or other numbering schemes.

Software programs and bioinformatical tools, such as ABodyBuilder and Paratome can also be used to determine CDR sequences. Additionally, delineation of a CDR can be according to X-ray crystallography.

Definitive delineation of a CDR and identification of residues including the binding site of an antibody can be accomplished by solving the structure of the antibody and/or solving the structure of the antibody-epitope complex. In particular embodiments, this can be accomplished by methods such as X-ray crystallography. Alternatively, CDRs are determined by comparison to known antibodies (linear sequence) and without resorting to solving a crystal structure. To determine residues involved in binding, a co-crystal structure of the Fab (antibody fragment) bound to the target can optionally be determined. Software programs, such as ABodyBuilder can also be used.

The carboxy-terminal portion of each chain defines a constant region, which can be responsible for effector function particularly in the heavy chain (the Fc). Examples of effector functions include: C1q binding and complement dependent cytotoxicity (CDC); antibody-dependent cell-mediated cytotoxicity (ADCC); phagocytosis; down regulation of cell surface receptors (e.g., B-cell receptors); and B-cell activation.

Within full-length light and heavy chains, the variable and constant regions are joined by a “J” region of amino acids, with the heavy chain also including a “D” region of amino acids. See, e.g., Fundamental Immunology, Ch. 7 (Paul, W., ed., 2nd ed. Raven Press, N.Y. (1989)).

Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, IgG1, IgG2, IgG3, and IgG4. IgM has subclasses including IgM1 and IgM2. IgA is similarly subdivided into subclasses including IgA1 and IgA2.

As indicated, antibodies bind epitopes on antigens. The term antigen refers to a molecule or a portion of a molecule capable of being bound by an antibody. An epitope is a region of an antigen that is bound by the variable region of an antibody. Epitope determinants can include chemically active surface groupings of molecules such as amino acids, sugar side chains, phosphoryl or sulfonyl groups, and can have specific three-dimensional structural characteristics, and/or specific charge characteristics. When the antigen is a protein or peptide, the epitope includes specific amino acids within that protein or peptide that contact the variable region of an antibody.

Unless otherwise indicated, the term “antibody” includes (in addition to antibodies having two full-length heavy chains and two full-length light chains as described above) variants, derivatives, and fragments thereof, examples of which are described below. Furthermore, unless explicitly excluded, antibodies can include monoclonal antibodies, human or humanized antibodies, antibodies with multiple binding domains, bispecific antibodies, trispecific antibodies, tetraspecific antibodies, multi-specific antibodies, polyclonal antibodies, linear antibodies, minibodies, domain antibodies, synthetic antibodies, chimeric antibodies, antibody fusions, and fragments thereof, respectively. In particular embodiments, antibodies can include oligomers or multiplexed versions of antibodies.

A monoclonal antibody refers to an antibody obtained from a population of substantially homogeneous antibodies, i.e., the individual antibodies including the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which include different antibodies directed against different epitopes, each monoclonal antibody of a monoclonal antibody preparation is directed against a single epitope on an antigen. Thus, the modifier “monoclonal” indicates the character of the antibody as being obtained from a substantially homogeneous population of antibodies and is not to be construed as requiring production of the antibody by any particular method. For example, monoclonal antibodies can be made by a variety of techniques, including the hybridoma method, recombinant DNA methods, phage-display methods, and methods utilizing transgenic animals containing all or part of the human immunoglobulin loci.

A “human antibody” is one which includes an amino acid sequence which corresponds to that of an antibody produced by a human or a human cell or derived from a non-human source that utilizes human antibody repertoires or other human antibody-encoding sequences.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. The subgroup of sequences can be a subgroup as in Kabat et al., Sequences of Proteins of Immunological Interest, Fifth Edition, NIH Publication 91-3242, Bethesda Md. (1991), vols. 1-3. In particular embodiments, for the V_{L}, the subgroup can be subgroup kappa I as in Kabat et al. (supra). In particular embodiments, for the V_{H}, the subgroup can be subgroup Ill as in Kabat et al. (supra).

A “humanized” antibody refers to a chimeric antibody including amino acid residues from non-human CDRs and amino acid residues from human FRs. In particular embodiments, a humanized antibody will include substantially all of at least one, and typically two, variable domains, in which all or substantially all of the CDRs correspond to those of a non-human antibody, and all or substantially all of the FRs correspond to those of a human antibody. A humanized antibody optionally may include at least a portion of an antibody constant region derived from a human antibody. A “humanized form” of an antibody, e.g., a non-human antibody, refers to an antibody that has undergone humanization.

Humanized antibodies and methods of making them are reviewed, e.g., in Almagro and Fransson, Front. Biosci. 13:1619-1633, 2008, and are further described, e.g., in Riechmann et al., Nature 332:323-329, 1988; Queen et al., Proc. Nat'l Acad. Sci. USA 86:10029-10033, 1989; U.S. Pat. Nos. 5,821,337, 7,527,791, 6,982,321, and 7,087,409; Kashmiri et al., Methods 36:25-34, 2005 (describing SDR (a-CDR) grafting); Padlan, Mol. Immunol. 28:489-498, 1991 (describing “resurfacing”); Dall'Acqua et al., Methods 36:43-60,2005 (describing “FR shuffling”); and Osbourn et al., Methods 36:61-68, 2005 and Klimka et al., Br. J. Cancer, 83:252-260, 2000 (describing the “guided selection” approach to FR shuffling). EP-B-0239400 provides additional description of “CDR-grafting”, in which one or more CDR sequences of a first antibody is/are placed within a framework of sequences not of that antibody, for instance of another antibody.

Human framework regions that may be used for humanization include: framework regions selected using the “best-fit” method (see, e.g., Sims et al. J. Immunol. 151:2296, 1993); framework regions derived from the consensus sequence of human antibodies of a particular subgroup of light or heavy chain variable regions (see, e.g., Carter et al., Proc. Nati. Acad. Sci. USA, 89:4285, 1992; and Presta et al., J. Immunol., 151:2623, 1993); human mature (somatically mutated) framework regions or human germline framework regions (see, e.g., Almagro and Fransson, Front. Biosci. 13:1619-1633, 2008); and framework regions derived from screening FR libraries (see, e.g., Baca et al., J. Biol. Chem. 272:10678-10684, 1997; and Rosok et al., J. Biol. Chem. 271:22611-2261 8, 1996).

Referring to the antibodies provided herein, the variable heavy chain CDR encoding sequences are provided as follows:

TABLE 1

| | | | Position in | SEQ ID |
|---|---|---|---|---|
| Antibody | CDR | SEQUENCE | VH or VL | NO: |
| CD90 | CDRH1 | GGCTACACCTTCACCAGTTACTGG | 76-99 | 2 |
| 5E10 | CDRH2 | ATTTTTCCTTCTGACAGTCATACT | 151-174 | 3 |
| naïve | CDRH3 | TACGAGGGACTTCGATACCCAGTCTCATG | 287-328 | 4 |
| (IMGT) | | CTATGGAATACT | | |
| CD90 | CDRH1 | CTGGATAAACTGG | 96-108 | 5 |
| 5E10 | CDRH2 | ATTTTTCCTTCTGACAGTCATACTAATTAC | 150-196 | 6 |
| naïve | | AATCAAAAATTCAAGG | | |
| (Kabat) | CDRH3 | TCGATACCCAGTTCTATGCTATGGAATAC | 293-328 | 7 |
| | | T | | |
| CD90 | CDRH1 | CTACACCTTCACCAGTTACTGG | 78-99 | 8 |
| 5E10 | CDRH2 | CCTTCTGACAGTCAT | 156-171 | 9 |
| naïve | CDRH3 | TCGATACCCAGTTCTATGCTATGGAATAC | 293-328 | 7 |
| (Chothia) | | T | | |
| CD90 | CDRH1 | ACTTCTGGCTACACCTTCACCAGTTACTG | 69-108 | 10 |
| 5E10 | | GATAAACTGG | | |
| naïve | CDRH2 | AATTTTTCCTTCTGACAGTCATACTAAT | 150-177 | 11 |
| (North) | CDRH3 | TACGAGGGACTTCGATACCCAGTCTCATG | 287-328 | 4 |
| | | CTATGGAATACT | | |
| CD90 | CDRH1 | GGATATACGTTTACCTCCTACTGG | 76-99 | 170 |
| 5E10 | CDRH2 | ATTTTCCCCAGTGATTCCCACACT | 151-174 | 23 |
| codon- | CDRH3 | ACAAGAGATTTCGACACGCAGTTCTACGC | 289-327 | 166 |
| optimized | | TATGGAATAC | | |
| (IMGT) | | | | |
| CD90 | CDRH1 | TGGATCAACTGG | 96-108 | 24 |
| 5E10 | CDRH2 | ATTTTCCCCAGTGATTCCCACACTAACTAT | 150-196 | 25 |
| codon- | | AATCAAAAATTTAAGG | | |
| optimized | CDRH3 | GAGATTTCGACACGCAGTTCTACGCTATG | 293-328 | 26 |
| (Kabat) | | GAATACT | | |
| CD90 | CDRH1 | TATACGTTTACCTCCTACTGG | 78-99 | 27 |
| 5E10 | CDRH2 | CCCAGTGATTCCCAC | 156-171 | 28 |
| codon- | CDRH3 | GAGATTTCGACACGCAGTTCTACGCTATG | 293-328 | 26 |
| optimized | | GAATACT | | |
| (Chothia) | | | | |
| CD90 | CDRH1 | ACGAGTGGATATACGTTTACCTCCTACTG | 69-108 | 29 |
| 5E10 | | GATCAACTGG | | |
| codon- | CDRH2 | ATTTTCCCCAGTGATTCCCACACTAAC | 150-177 | 30 |
| optimized | CDRH3 | ACAAGAGATTTCGACACGCAGTTCTACGC | 289-327 | 166 |
| (North) | | TATGGAATAC | | |

Referring to the antibodies provided herein, the variable light chain CDR encoding sequences are provided as follows:

TABLE 2

| | | | Position in | SEQ ID |
|---|---|---|---|---|
| Antibody | CDR | SEQUENCE | VH or VL | NO: |
| CD90 | CDRL1 | GCCATCTCAAGTGTAAATTGC | 73-93 | 167 |
| 5E10 naïve | CDRL2 | TTATGCAACATCC | 141-153 | 14 |
| (IMGT) | CDRL3 | CATCAGTGGAGTAGTTACCCACCAACG | 262-288 | 15 |
| CD90 | CDRL1 | GCACTGGTACCAG | 96-108 | 16 |
| 5E10 naïve | CDRL2 | ATCCAAACTGACTCTTGGAGTCCCTGCTT | 150-198 | 17 |
| (Kabat) | | GCTTCAGTGGCAGTGGGTCT | | |
| | CDRL3 | CATCAGTGGAGTAGTTACCCACCAACG | 262-288 | 15 |
| CD90 | CDRL1 | CTCAAGTGTAAATTGCATGCAC | 78-99 | 18 |
| 5E10 naïve | CDRL2 | CTGACTCTTGGAGTC | 156-171 | 19 |
| (Chothia) | CDRL3 | CATCAGTGGAGTAGTTACCCACCAACG | 262-288 | 15 |
| CD90 | CDRL1 | AGTGCCATCTCAAGTGTAAATTGCATGCA | 69-108 | 20 |
| 5E10 naïve | | CTGGTACCAG | | |
| (North) | CDRL2 | ATCCAAACTGACTCTTGGAGTCCCTGCT | 150-177 | 21 |
| | CDRL3 | CATCAGTGGAGTAGTTACCCACCAACG | 262-288 | 15 |
| CD90 | CDRL1 | GCCATTAGTAGCGTGAATTGC | 73-93 | 169 |
| 5E10 | CDRL2 | ATATGCTACTAGT | 141-153 | 32 |
| codon- | CDRL3 | CACCAGTGGTCTTCTTATCCTCCTACA | 262-288 | 33 |
| optimized | | | | |
| (IMGT) | | | | |
| CD90 | CDRL1 | TGTGCCATCTCAAGTGTAAATTACATGCA | 70-105 | 34 |
| 5E10 | | C | | |
| codon- | CDRL2 | TAGTAAGCTGACCCTCGGAGTACCGGCTT | 150-196 | 35 |
| optimized | | GTTTTAGTGGAAGTGGAT | | |
| (Kabat) | CDRL3 | TCGATACCCAGTTCTATGCTATGGAATAC | 293-328 | 7 |
| | | T | | |
| CD90 | CDRL1 | TGTGCCATCTCAAGTGTAAATTACATGCA | 70-105 | 34 |
| 5E10 | | C | | |
| codon- | CDRL2 | GCTGACCCTCGGAGTA | 156-171 | 37 |
| optimized | CDRL3 | TCGATACCCAGTTCTATGCTATGGAATAC | 293-328 | 7 |
| (Chothia) | | T | | |
| CD90 | CDRL1 | TGTGCCATCTCAAGTGTAAATTACATGCA | 70-105 | 34 |
| 5E10 | | C | | |
| codon- | CDRL2 | TAGTAAGCTGACCCTCGGAGTACCGGCT | 150-177 | 39 |
| optimized | CDRL3 | TACGAGGGACTTCGATACCCAGTCTCATG | 287-328 | 4 |
| (North) | | CTATGGAATACT | | |
| CD90 5E10 | CDRL1 | GCCATTAGTAGCGTGAATTAC | 73-93 | 168 |
| mutated Cys | CDRL2 | ATATGCTACTAGT | 151-174 | 32 |
| (IMGT) | CDRL3 | CACCAGTGGTCTTCTTATCCTCCTACA | 262-288 | 33 |
| CD90 5E10 | CDRL1 | TGTGCCATCTCAAGTGTAAATTACATGCA | 70-105 | 34 |
| mutated Cys | | C | | |
| (Kabat) | CDRL2 | TAGTAAGCTGACCCTCGGAGTACCGGCTT | 150-196 | 35 |
| | | GTTTTAGTGGAAGTGGAT | | |
| | CDRL3 | TCGATACCCAGTTCTATGCTATGGAATAC | 293-328 | 7 |
| | | T | | |
| CD90 5E10 | CDRL1 | TGTGCCATCTCAAGTGTAAATTACATGCA | 70-105 | 34 |
| mutated Cys | | C | | |
| (Chothia) | CDRL2 | GCTGACCCTCGGAGTA | 156-171 | 37 |
| | CDRL3 | TCGATACCCAGTTCTATGCTATGGAATAC | 293-328 | 7 |
| | | T | | |
| CD90 5E10 | CDRL1 | TGTGCCATCTCAAGTGTAAATTACATGCA | 70-105 | 34 |
| mutated Cys | | C | | |
| (North) | CDRL2 | TAGTAAGCTGACCCTCGGAGTACCGGCT | 150-177 | 39 |
| | CDRL3 | TACGAGGGACTTCGATACCCAGTCTCATG | 287-328 | 4 |
| | | CTATGGAATACT | | |

The heavy chain of the CD90 5E10 naïve, codon-optimized, and mutated Cys sequences all encode the CD90 5E10 heavy chain sequences provided below. The light chain of the CD90 5E10 naïve and codon-optimized sequences encode the CD90 5E10 light chain sequence provided below. The light chain of the CD90 5E10 mutated Cys sequence encodes the CD90 5E10 mutated Cys sequence as provided below.

TABLE 3

| | | | Position in | SEQ ID |
|---|---|---|---|---|
| Antibody | CDR | SEQUENCE | VH or VL | NO: |
| CD90 5E10 | CDRH1 | GYTFTSYW | 26-34 | 41 |
| (IMGT) | CDRH2 | IFPSDSHT | 51-58 | 42 |
| | CDRH3 | TRDFDTQFYAMEY | 97-109 | 43 |
| CD90 5E10 | CDRH1 | SYWIN | 32-36 | 44 |
| (Kabat) | CDRH2 | KIFPSDSHTNYNQKFKD | 50-66 | 45 |
| | CDRH3 | DFDTQFYAMEY | 99-109 | 46 |
| CD90 5E10 | CDRH1 | GYTFTSY | 26-33 | 47 |
| (Chothia) | CDRH2 | FPSDSH | 52-57 | 48 |
| | CDRH3 | DFDTQFYAMEY | 99-109 | 46 |
| CD90 5E10 | CDRH1 | KTSGYTFTSYWIN | 23-36 | 49 |
| (North) | CDRH2 | KIFPSDSHTN | 50-59 | 50 |
| | CDRH3 | TRDFDTQFYAMEY | 97-109 | 43 |
| CD90 5E10 | CDRL1 | AISSVNC | 25-31 | 53 |
| (IMGT) | CDRL2 | ATS | 49-51 | N/A |
| | CDRL3 | HQWSSYPPT | 88-96 | 54 |
| CD90 5E10 | CDRL1 | SAISSVNCMH | 24-33 | 55 |
| (Kabat) | CDRL2 | ATSKLTL | 49-55 | 56 |
| | CDRL3 | HQWSSYPPT | 88-96 | 54 |
| CD90 5E10 | CDRL1 | SAISSVNCMH | 24-33 | 55 |
| (Chothia) | CDRL2 | ATSKLTL | 49-55 | 56 |
| | CDRL3 | HQWSSYPPT | 88-96 | 54 |
| CD90 5E10 | CDRL1 | SAISSVNCHM | 24-33 | 172 |
| (North) | CDRL2 | YATSKLTL | 49-55 | 58 |
| | CDRL3 | HQWSSYPPT | 88-96 | 54 |
| CD90 5E10 mutated | CDRL1 | AISSVNY | 25-31 | 171 |
| unpaired Cys | CDRL2 | ATS | 49-51 | N/A |
| (IMGT) | CDRL3 | HQWSSYPPT | 88-96 | 54 |
| CD90 5E10 mutated | CDRL1 | SAISSVNYMH | 24-33 | 173 |
| unpaired Cys | CDRL2 | ATSKLTL | 49-55 | 56 |
| (Kabat) | CDRL3 | HQWSSYPPT | 88-96 | 54 |
| CD90 5E10 mutated | CDRL1 | SAISSVNYMH | 24-33 | 173 |
| unpaired Cys | CDRL2 | ATSKLTL | 49-55 | 56 |
| (Chothia) | CDRL3 | HQWSSYPPT | 88-96 | 54 |
| CD90 5E10 mutated | CDRL1 | SAISSVNYHM | 24-33 | 174 |
| unpaired Cys | CDRL2 | YATSKLTL | 49-55 | 58 |
| (North) | CDRL3 | HQWSSYPPT | 88-96 | 54 |

In particular embodiments, a naïve CD90 5E10 sequence includes a variable heavy chain encoded by the sequence as set forth in SEQ ID NO: 1 and a variable light chain encoded by the sequence as set forth in SEQ ID NO: 12.

In particular embodiments, a codon-optimized CD90 5E10 sequence includes a variable heavy chain encoded by the sequence as set forth in SEQ ID NO: 22 and a variable light chain encoded by the sequence as set forth in SEQ ID NO: 31.

In particular embodiments, a CD90 5E10 antibody includes a variable heavy chain including the sequence as set forth in SEQ ID NO: 40 and a variable light chain including the sequence as set forth in SEQ ID NO: 52.

In particular embodiments, a codon-optimized CD90 5E10 sequence with mutated unpaired cysteines includes a variable heavy chain encoded by the sequence as set forth in SEQ ID NO: 22 and a variable light chain encoded by the sequence as set forth in SEQ ID NO: 144.

In particular embodiments, a codon-optimized CD90 5E10 antibody with mutated unpaired cysteines includes a variable heavy chain including the sequence as set forth in SEQ ID NO: 40 and a variable light chain including the sequence as set forth in SEQ ID NO: 145.

Antibodies disclosed herein can be utilized to prepare various forms of relevant binding domain molecules. For example, particular embodiments can include binding fragments of an antibody, e.g., Fv, Fab, Fab′, F(ab′)₂, and single chain Fv fragments (scFvs) or any biologically effective fragments of an immunoglobulin that bind specifically to an epitope described herein.

In particular embodiments, an antibody fragment is used. An “antibody fragment” denotes a portion of a complete or full-length antibody that retains the ability to bind to an epitope. Antibody fragments can be made by various techniques, including proteolytic digestion of an intact antibody as well as production by recombinant host-cells (e.g., mammalian suspension cell lines, E. coli or phage), as described herein. Antibody fragments can be screened for their binding properties in the same manner as intact antibodies. Examples of antibody fragments include Fv, scFv, Fab, Fab′, Fab′-SH, F(ab′)₂; diabodies; and linear antibodies.

A single chain variable fragment (scFv) is a recombinant protein of the variable regions of the heavy and light chains of immunoglobulins connected with a linker peptide. Fv fragments include the V_{L} and V_{H} domains of a single arm of an antibody but lack the constant regions. Although the two domains of the Fv fragment, V_{L} and V_{H}, are coded by separate genes, they can be joined, using, for example, recombinant methods, by a synthetic linker that enables them to be made as a single protein chain in which the V_{L} and V_{H} regions pair to form monovalent molecules (single chain Fv (scFv)). For additional information regarding Fv and scFv, see e.g., Bird, et al., Science 242:423-426, 1988; Huston, et al., Proc. Natl. Acad. Sci. USA 85:5879-5883, 1988; Plueckthun, in The Pharmacology of Monoclonal Antibodies, vol. 113, Rosenburg and Moore (eds.), Springer-Verlag, New York), (1994) 269-315; WO 1993/16185; U.S. Pat. Nos. 5,571,894; and 5,587,458.

Linker sequences that are used to connect the VL and VH of an scFv or other forms of binding fragments are generally five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies. Linker sequences of scFv are commonly Gly-Ser linkers. In particular embodiments, the linker sequence includes the sequence: GGGGSGGGGSGGGGS (SEQ ID NO: 64) or GSDSNAGHASAGNTS (SEQ ID NO: 66). Additional linker sequences that can be used include sets of glycine and serine repeats such as from one to ten repeats of (GlyₓSer_{y})ₙ, wherein x and y are independently an integer from 0 to 10 provided that x and y are not both 0 and wherein n is an integer of 1, 2, 3, 4, 5, 6, 7, 8, 9 or 10). Particular examples include (Gly₄Ser)ₙ (SEQ ID NO: 79), (Gly₃Ser)ₙ(Gly₄Ser)ₙ (SEQ ID NO: 80), (Gly₃Ser)ₙ(Gly₂Ser)ₙ (SEQ ID NO: 81), or (Gly₃Ser)ₙ(Gly₄Ser)₁ (SEQ ID NO: 82). In particular embodiments, the linker is (Gly₄Ser)₄ (SEQ ID NO: 83), (Gly₄Ser)₂ (SEQ ID NO: 84), (Gly₄Ser)₁ (SEQ ID NO: 85), (Gly₃Ser)₂ (SEQ ID NO: 86), (Gly₃Ser)₁ (SEQ ID NO: 87), (Gly₂Ser)₂ (SEQ ID NO: 88) or (Gly₂Ser)₁, GGSGGGSGGSG (SEQ ID NO: 89), GGSGGGSGSG (SEQ ID NO: 90), or GGSGGGSG (SEQ ID NO: 91).

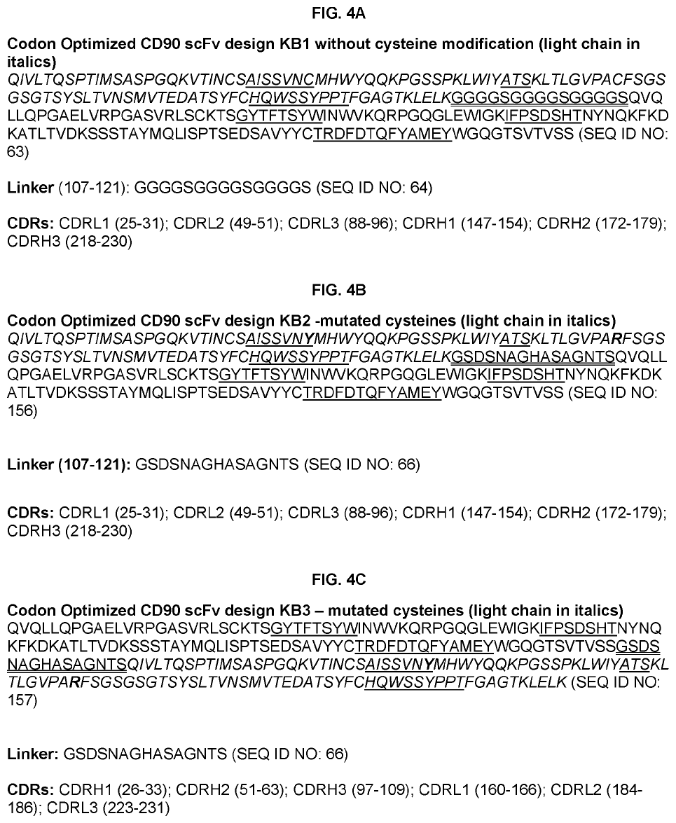

A particular example of an anti-CD90 scFv is shown in FIG. 4A (SEQ ID NO: 63). This scFv, referred to herein as KB1, includes (from N to C terminal) a CD90 5E10 light chain, GGGGSGGGGSGGGGS (SEQ ID NO: 64) linker, and a CD905E10 heavy chain.

Another particular example of an anti-CD90 scFv is shown in FIG. 4B (SEQ ID NO: 156). This scFv, referred to herein as KB2, includes (from N to C terminal) a CD90 5E10 light chain wherein the unpaired cysteines are mutated, GSDSNAGHASAGNTS (SEQ ID NO: 66) linker, and a CD905E10 heavy chain.

Another particular example of an anti-CD90 scFv is shown in FIG. 4C (SEQ ID NO: 157). This scFv, referred to herein as KB3, includes (from N to C terminal) a CD90 5E10 heavy chain, GSDSNAGHASAGNTS (SEQ ID NO: 66) linker, and a CD905E10 light chain wherein the unpaired cysteines are mutated.

Additional examples of antibody-based binding domain formats include scFv-based grababodies and soluble VH domain antibodies. These antibodies form binding regions using only heavy chain variable regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

A Fab fragment is a monovalent antibody fragment including V_{L}, V_{H}, CL and CH1 domains. A F(ab′)₂ fragment is a bivalent fragment including two Fab fragments linked by a disulfide bridge at the hinge region. For discussion of Fab and F(ab′)₂ fragments having increased in vivo half-life, see U.S. Pat. No. 5,869,046. Diabodies include two epitope-binding sites that may be bivalent. See, for example, EP 0404097; WO1993/01161; and Holliger, et al., Proc. Natl. Acad. Sci. USA 90:6444-6448, 1993. Dual affinity retargeting antibodies (DART™; based on the diabody format but featuring a C-terminal disulfide bridge for additional stabilization (Moore et al., Blood 117:4542-51, 2011)) can also be used. Antibody fragments can also include isolated CDRs. For a review of antibody fragments, see Hudson, et al., Nat. Med. 9:129-134, 2003.

Variants of antibodies described herein are also included. Variants of antibodies can include those having one or more conservative amino acid substitutions or one or more non-conservative substitutions that do not adversely affect the binding of the protein. Options for conservative substitutions are described elsewhere herein.

In particular embodiments, a V_{L} region can be derived from or based on a disclosed V_{L} and can include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the disclosed V_{L}. An insertion, deletion or substitution may be anywhere in the V_{L} region, including at the amino- or carboxy-terminus or both ends of this region, provided that each CDR includes zero changes or at most one, two, or three changes and provided an antibody including the modified V_{L} region can still specifically bind its target epitope with an affinity similar to the wild type binding domain.

In particular embodiments, a V_{H} region can be derived from or based on a disclosed V_{H} and can include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the V_{H} disclosed herein. An insertion, deletion or substitution may be anywhere in the V_{H} region, including at the amino- or carboxy-terminus or both ends of this region, provided that each CDR includes zero changes or at most one, two, or three changes and provided an antibody including the modified V_{H} region can still specifically bind its target epitope with an affinity similar to the wild type binding domain.

In particular embodiments, one or more amino acid modifications may be introduced into the Fc region of an antibody, thereby generating an Fc region variant. The Fc region variant may include a human Fc region sequence (e.g., a human IgG1, IgG2, IgG3 or IgG4 Fc region) including an amino acid modification (e.g., a substitution) at one or more amino acid positions.

In particular embodiments, variants have been modified from a reference sequence to produce an administration benefit. Exemplary administration benefits can include (1) reduced susceptibility to proteolysis, (2) reduced susceptibility to oxidation, (3) altered binding affinity for forming protein complexes, (4) altered binding affinities, (5) reduced immunogenicity; and/or (6) extended half-live. While the disclosure below describes these modifications in terms of their application to antibodies, when applicable to another particular anti-CD90 binding domain format (e.g., an scFv, bispecific antibodies), the modifications can also be applied to these other formats.

In particular embodiments the antibodies can be mutated to increase their affinity for Fc receptors. Exemplary mutations that increase the affinity for Fc receptors include: G236A/S239D/A330L/1332E (GASDALIE). Smith et al., Proceedings of the National Academy of Sciences of the United States of America, 109(16), 6181-6186, 2012. In particular embodiments, an antibody variant includes an Fc region with one or more amino acid substitutions which improve ADCC, e.g., substitutions at positions 298, 333, and/or 334 of the Fc region (EU numbering of residues). In particular embodiments, alterations are made in the Fc region that result in altered C1q binding and/or Complement Dependent Cytotoxicity (CDC), e.g., as described in U.S. Pat. No. 6,194,551, WO 99/51642, and Idusogie et al., J. Immunol. 164: 4178-4184, 2000.

In particular embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further below. In particular embodiments, residue 5400 (EU numbering) of the heavy chain Fc region is selected. Cysteine engineered antibodies may be generated as described, e.g., in U.S. Pat. No. 7,521,541.

Antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at position 297 in the Fc region (Eu numbering of Fc region residues); however, Asn297 may also be located ±3 amino acids upstream or downstream of position 297, i.e., between positions 294 and 300, due to minor sequence variations in antibodies. Such fucosylation variants may have improved ADCC function. See, e.g., WO2000/61739; WO 2001/29246; WO2002/031140; US2002/0164328; WO2003/085119; WO2003/084570; US2003/0115614; US2003/0157108; US2004/0093621; US2004/0110704; US2004/0132140; US2004/0110282; US2004/0109865; WO2005/035586; WO2005/035778; WO2005/053742; Okazaki et al. J. Mol. Biol. 336:1239-1249 (2004); and Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545, 1986, and knockout cell lines, such as alpha-1,6-fucosyltransferase gene, FUT8, knockout CHO cells (see, e.g., Yamane-Ohnuki et al., Biotech. Bioeng. 87: 614, 2004; Kanda et al., Biotechnol. Bioeng., 94(4):680-688, 2006; and WO2003/085107).

In particular embodiments, modified antibodies include those wherein one or more amino acids have been replaced with a non-amino acid component, or where the amino acid has been conjugated to a functional group or a functional group has been otherwise associated with an amino acid. The modified amino acid may be, e.g., a glycosylated amino acid, a PEGylated amino acid, a farnesylated amino acid, an acetylated amino acid, a biotinylated amino acid, an amino acid conjugated to a lipid moiety, or an amino acid conjugated to an organic derivatizing agent. Amino acid(s) can be modified, for example, co-translationally or post-translationally during recombinant production (e.g., N-linked glycosylation at N-X-S/T motifs during expression in mammalian cells) or modified by synthetic means. The modified amino acid can be within the sequence or at the terminal end of a sequence. Modifications also include nitrited constructs.

In particular embodiments, variants include glycosylation variants wherein the number and/or type of glycosylation site has been altered compared to the amino acid sequences of a reference sequence. In particular embodiments, glycosylation variants include a greater or a lesser number of N-linked glycosylation sites than the reference sequence. An N-linked glycosylation site is characterized by the sequence: Asn-X-Ser or Asn-X-Thr, wherein the amino acid residue designated as X can be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (e.g., those that are naturally occurring) are eliminated and one or more new N-linked sites are created. Additional antibody variants include cysteine variants wherein one or more cysteine residues are deleted from or substituted for another amino acid (e.g., serine) as compared to the reference sequence. These cysteine variants can be useful when antibodies must be refolded into a biologically active conformation such as after the isolation of insoluble inclusion bodies. These cysteine variants generally have fewer cysteine residues than the reference sequence, and typically have an even number to minimize interactions resulting from unpaired cysteines.

PEGylation particularly is a process by which polyethylene glycol (PEG) polymer chains are covalently conjugated to other molecules such as proteins. Several methods of PEGylating proteins have been reported in the literature. For example, N-hydroxy succinimide (NHS)-PEG was used to PEGylate the free amine groups of lysine residues and N-terminus of proteins; PEGs bearing aldehyde groups have been used to PEGylate the amino-termini of proteins in the presence of a reducing reagent; PEGs with maleimide functional groups have been used for selectively PEGylating the free thiol groups of cysteine residues in proteins; and site-specific PEGylation of acetyl-phenylalanine residues can be performed.

Covalent attachment of proteins to PEG has proven to be a useful method to increase the half-lives of proteins in the body (Abuchowski, A. et al., Cancer Biochem. Biophys., 1984, 7:175-186; Hershfield, M. S. et al., N. Engl. J. Medicine, 1987, 316:589-596; and Meyers, F. J. et al., Clin. Pharmacol. Ther., 49:307-313, 1991). The attachment of PEG to proteins not only protects the molecules against enzymatic degradation, but also reduces their clearance rate from the body. The size of PEG attached to a protein has significant impact on the half-life of the protein. The ability of PEGylation to decrease clearance is generally not a function of how many PEG groups are attached to the protein, but the overall molecular weight of the altered protein. Usually the larger the PEG is, the longer the in vivo half-life of the attached protein. In addition, PEGylation can also decrease protein aggregation (Suzuki et al., Biochem. Bioph. Acta 788:248, 1984), alter protein immunogenicity (Abuchowski et al., J. Biol. Chem. 252: 3582, 1977), and increase protein solubility as described, for example, in PCT Publication No. WO 92/16221).

Several sizes of PEGs are commercially available (Nektar Advanced PEGylation Catalog 2005-2006; and NOF DDS Catalogue Ver 7.1), which are suitable for producing proteins with targeted circulating half-lives. A variety of active PEGs have been used including mPEG succinimidyl succinate, mPEG succinimidyl carbonate, and PEG aldehydes, such as mPEG-propionaldehyde.

In particular embodiments, anti-CD90 antibodies or anti-CD90 binding fragments disclosed herein are conjugated to polyglutamic acid (PGA). Antibody-functionalized PGA can be useful because it shields the positive charge of polymeric nanoparticles (Moffett et al., Nature Communications 8 (389, 2017). PGA can be functionalized with antibodies using similar covalent attachment methods described for PEGylation.

In particular embodiments, the antibody can be fused or coupled to an Fc polypeptide that includes amino acid alterations that extend the in vivo half-life of an antibody that contains the altered Fc polypeptide as compared to the half-life of a similar antibody containing the same Fc polypeptide without the amino acid alterations. In particular embodiments, Fc polypeptide amino acid alterations can include M252Y, S254T, T256E, M428L, and/or N434S and can be used together, separately or in any combination. For example, M428L/N434S is a pair of mutations that increase the half-life of antibodies in serum, as described in Zalevsky et al., Nature Biotechnology 28, 157-159, 2010. Other alterations that can be helpful are described in U.S. Pat. Nos. 7,083,784, 7,670,600, US Publication No. 2010/0234575, PCT/US2012/070146, and Zwolak, Scientific Reports 7: 15521, 2017. In particular embodiments, any substitution at one of the following amino acid positions in an Fc polypeptide can be considered an Fc alteration that extends half-life: 250, 251, 252, 259, 307, 308, 332, 378, 380, 428, 430, 434, 436. Each of these alterations or combinations of these alterations can be used to extend the half-life of an antibody as described herein.

In particular embodiments, antibodies disclosed herein are formed using the Daedalus expression system as described in Pechman et al. (Am J Physiol 294: R1234-R1239, 2008). The Daedalus system utilizes inclusion of minimized ubiquitous chromatin opening elements in transduction vectors to reduce or prevent genomic silencing and to help maintain the stability of decigram levels of expression. This system can bypass tedious and time-consuming steps of other protein production methods by employing the secretion pathway of serum-free adapted human suspension cell lines, such as 293 Freestyle. Using optimized lentiviral vectors, yields of 20-100 mg/I of correctly folded and post-translationally modified, endotoxin-free protein of up to 70 kDa in size, can be achieved in conventional, small-scale (100 ml) culture. At these yields, most proteins can be purified using a single size-exclusion chromatography step, immediately appropriate for use in structural, biophysical or therapeutic applications. Bandaranayake et al., Nucleic Acids Res., 39(21) 2011. In some instances, purification by chromatography may not be needed due to the purity of manufacture according the methods described herein.

### (ii) Use of CD90 Antibodies & Binding Fragments Thereof to Enhance Cell Specificity of Viral Vector Delivery

Current virus-based vector systems can often have broad cell specificity. The current disclosure provides a system for and method of refining a viral vector's natural tropism to target CD90+ cells more specifically by incorporating an antibody or binding fragment thereof disclosed herein on the surface of a viral vector. In particular embodiments, the antibody or binding fragment thereof is part of a recombinant protein, expressed together with a viral structural protein. In certain examples, the viral structural protein is a viral attachment and/or viral fusion protein. This approach can be used with any viral vector delivery system.

“Lentivirus” refers to a genus of retroviruses that are capable of infecting dividing and non-dividing cells and typically produce high viral titers. Lentiviral vectors have been employed in gene therapy for a number of diseases. For example, hematopoietic gene therapies using lentiviral vectors or gamma retroviral vectors have been used for x-linked adrenoleukodystrophy and beta thalassaemia. See, e.g., Kohn et al., 2010 Clin Immunol. 135:247-254; Cartier et al., 2012 Meth. Enzymol. 507:187-198; and Cavazzana-Calvo et al., 2010. Nature 467:318-322. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

Retroviruses are a family of RNA viruses that have an enzyme, reverse transcriptase, capable of making a complementary DNA copy of the viral RNA, which then is integrated into a host cell's DNA. During replication the RNA is converted into DNA. Following infection of the cell a double-stranded molecule of DNA is generated from the two molecules of RNA which are carried in the viral particle by the molecular process known as reverse transcription. The DNA form becomes covalently integrated in the host cell genome as a provirus, from which viral RNAs are expressed with the aid of cellular and/or viral factors. The expressed viral RNAs are packaged into particles and released as infectious virion. Retroviruses have been classified in various ways but the nomenclature has been standardized in the last decade (see ICTVdB—The Universal Virus Database, v 4 on the World Wide Web (www) at ncbi.nlm.nih.gov/ICTVdb/ICTVdB/ and the text book “Retroviruses” Eds Coffin, Hughs and Varmus, Cold Spring Harbor Press 1997).

Foamy viruses (FVes) are the largest retroviruses known today and are widespread among different mammals, including all non-human primate species, however are absent in humans. FV vectors are suitable for gene therapy applications because they can (1) accommodate large transgenes (>9kb), (2) transduce slowly dividing cells efficiently, and (3) integrate as a provirus into the genome of target cells, thus enabling stable long-term expression of the transgene(s). FV vectors do need cell division for the pre-integration complex to enter the nucleus, however the complex is stable for at least 30 days and still infective. The intracellular half-life of the FV pre-integration complex is comparable to the one of lentiviruses and significantly higher than for gammaretroviruses, therefore FV are also—similar to LV vectors—able to transduce rarely dividing cells. FV vectors are natural self-inactivating vectors and characterized by the fact that they seem to have hardly any potential to activate neighboring genes. In addition, FV vectors can enter any cells known (although the receptor is not identified yet) and infectious vector particles can be concentrated 100-fold without loss of infectivity due to a stable envelope protein. FV vectors achieve high transduction efficiency in pluripotent HSC and have been used in animal models to correct monogenetic diseases such as leukocyte adhesion deficiency (LAD) in dogs and Fanconi anemia in mice. FV vectors are also used in preclinical studies of β-thalassemia.

Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Additional examples of viral vectors include those derived from adenoviruses (e.g., adenovirus 5 (Ad5), adenovirus 35 (Ad35), adenovirus 11 (Ad11), adenovirus 26 (Ad26), adenovirus 48 (Ad48) or adenovirus 50 (Ad50)), alphaviruses, cytomegaloviruses (CMV), flaviviruses, herpes viruses (e.g., herpes simplex), influenza viruses, papilloma viruses (e.g., human and bovine papilloma virus; see, e.g., U.S. Pat. No. 5,719,054), poxviruses, vaccinia viruses, etc. See Kozarsky and Wilson, 1993; Rosenfeld, et al., 1991; Rosenfeld, et al., 1992; Mastrangeli, et al., 1993; Walsh, et al., 1993; and Lundstrom, 1999. Examples include modified vaccinia Ankara (MVA) and NYVAC, or strains derived therefrom. Other examples include avipox vectors, such as a fowlpox vectors (e.g., FP9) or canarypox vectors (e.g., ALVAC and strains derived therefrom).

Viral vectors can be pseudotyped according to methods known to those of ordinary skill in the art. A pseudotyped viral vector refers to a viral vector particle bearing a structural protein (e.g., an envelope glycoprotein or capsid) derived from a virus distinct from the viral vector genome. Vector particles can be readily prepared by the skilled person, for example by following the general guidance provided by Sandrin et al. (2002) Blood 100:823-832. Briefly, the vector particles may be generated by co-expressing the packaging elements (i.e., the core and enzyme components), the genome component and the envelope component in a producer cell, e.g., 293T human embryonic kidney cells. Typically, from three to four plasmids may be employed, but the number may be greater depending upon the degree to which the lentiviral components are broken up into separate units. For the generation of a pseudotyped vector, the env gene, originally derived from the same virus as the gag and polgenes is exchanged for the structural protein(s) of a different virus. A method of generating a viral vector includes transfecting producer cells with plasmids. The plasmids include a transfer plasmid, one or more packaging plasmids, and an envelope (or structural) plasmid. After incubating in appropriate media, the packaging cells will produce virus and the virus can be harvested from the supernatant media. In particular embodiments, the transfer plasmid includes a 5′ long terminal repeat (LTR), a promoter, and a gene. The packaging plasmid contains the structural (gag), and replication (pol) genes which code for some of the proteins required to produce the viral vector. The pseudotyped protein is encoded by the envelope plasmid which contains a promoter, a sequence encoding the structural protein, and a polyA tail.

Herein, structural proteins refer to proteins (including glycoproteins) that make up the outermost layer of most viruses. A structural protein can include envelope proteins, membrane proteins, matrix proteins, attachment proteins, viral entry proteins, or other viral glycoproteins.

Measles is one of the most contagious viral diseases, and remains a major cause of childhood morbidity and mortality worldwide. The measles virus encodes at least eight structural proteins, which have letter names: F, C, H, L, M, N, P, and V. Of these proteins, H (hemagglutinin) has a role in the attachment of the virus to host cells, and F (fusion) is involved in the spread of the virus from one cell to another.

Vesicular stomatitis virus (VSV) is transmitted naturally by arthropods to a number of animal species including cattle, horses, and pigs. Vesicular stomatitis virus G (VSV-G) protein (accession number KT429217.1) is a type Ill viral fusion protein that mediates both cell attachment and membrane fusion.

Influenza viruses are orthomyxoviruses, members of the family Orthomyxoviridae, which include the Influenzavirus A, B and C, Thogotovirus, Quaranjavirus, and Isavirus. Influenza is an enveloped virus with structural proteins that can be found associated with the virus envelope, a lipid bilayer derived from the plasma membrane of the host cell. The viral envelope contains three of the viral transmembrane proteins: hemagglutinin (HΔ), neuraminidase (NA), and the matrix ion channel M2. HA (e.g., accession number AAA43099.1 or CAA40728.1) and NA proteins are the main proteins at the virus surface and HA is more abundant compared to NA.

Ebolavirus is an enveloped filamentous RNA virus that causes severe hemorrhagic fever. It enters the cell by macropinocytosis and membrane fusion in a late endosomal compartment. Fusion is mediated by the ebolavirus envelope glycoprotein GP, which includes subunits GP1 and GP2.

Coronaviruses (CoVs) are enveloped viruses with a positive sense, single-stranded RNA genome. The coronaviral genome encodes four major structural proteins: the spike (S) protein, nucleocapsid (N) protein, membrane (M) protein, and the envelope (E) protein, all of which are required to produce a structurally complete viral particle.

Lassa virus is an enveloped, single-stranded RNA virus. The two RNA segments in its genome encode four viral proteins, including zinc-binding protein (Z), RNA polymerase (L), nucleoprotein (NP), and the surface glycoprotein precursor (GP, or spike protein).

The Nipah virus envelope contains two surface glycoproteins required for cellular entry: the attachment protein (NiV-G) and fusion protein (NiV-F). Niv-G allows for attachment of the virion to cellular receptors. NiV-F mediates the fusion of the virus and cellular membranes.

Rabies virus is a rod- or bullet-shaped, single-stranded, negative-sense, unsegmented, enveloped RNA virus. The virus genome encodes five proteins: nucleoprotein (N), phosphoprotein (P), matrix protein (M), glycoprotein (G) and polymerase (L).

Respiratory syncytial virus (RSV) is an enveloped negative-sense single-stranded RNA virus. RSV encodes four envelope-associated proteins, of which three are membrane proteins: the small hydrophobic protein (SH), fusion protein (F), and attachment protein (G). The matrix protein (M) coordinates virion assembly.

Other structural proteins that could be used include HIV gp120, protein F of paramyxoviruses (accession number AAV54052.1), protein GP2 of filoviruses (accession number Q89853.1 or AAV48577.1), protein E of flaviviruses (accession number AAR87742.1), protein E1 of alphaviruses (e.g., accession number LC064744.1 [Chikungunya virus]), protein S of coronaviruses (accession number AAP33697.1 or BAC81404.1), protein gH of herpes simplex virus (accession number P06477), protein gB of herpes simplex virus (accession number P06437), and protein G2 of arenaviruses (accession number BAA00964.2 or P03540). Other structural proteins include Chikungunya (E1 Env and E2 Env), Hendra (F glycoprotein and G glycoprotein), hepatitis B (large (L), middle (M), and small (S)), hepatitis C (glycoprotein E1 and glycoprotein E2), HIV envelope (Env), among many others.

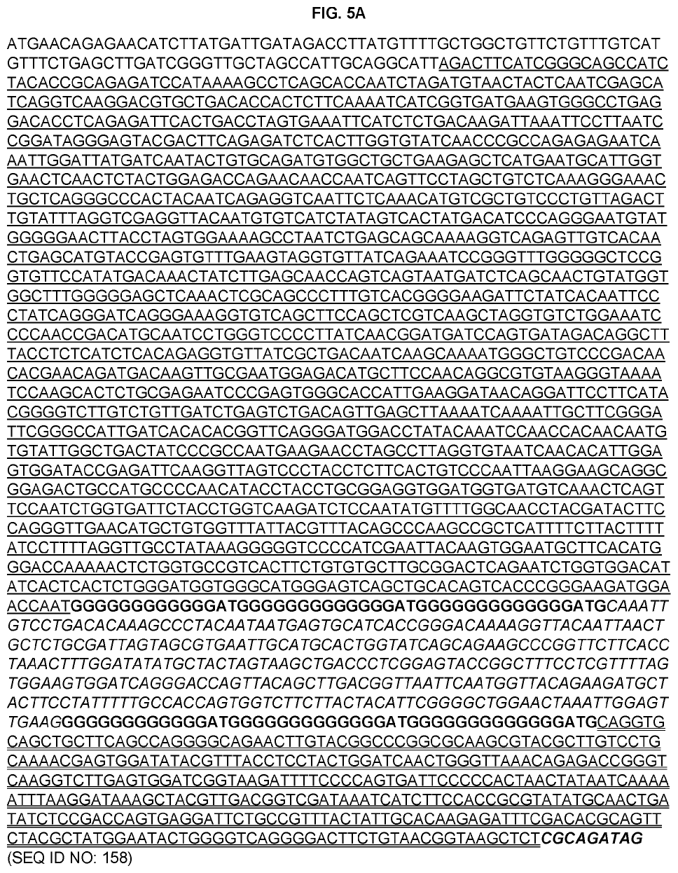

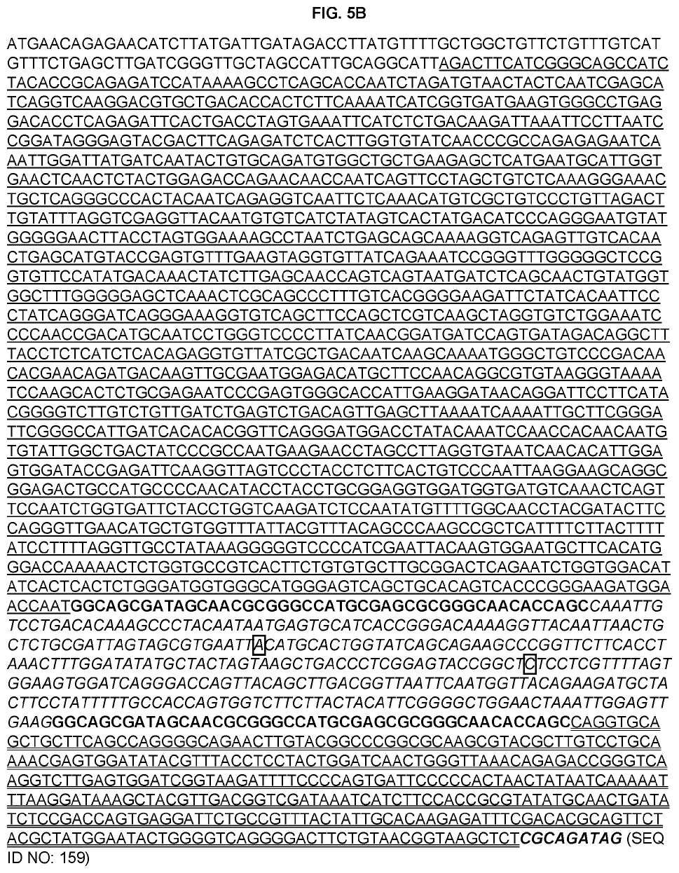

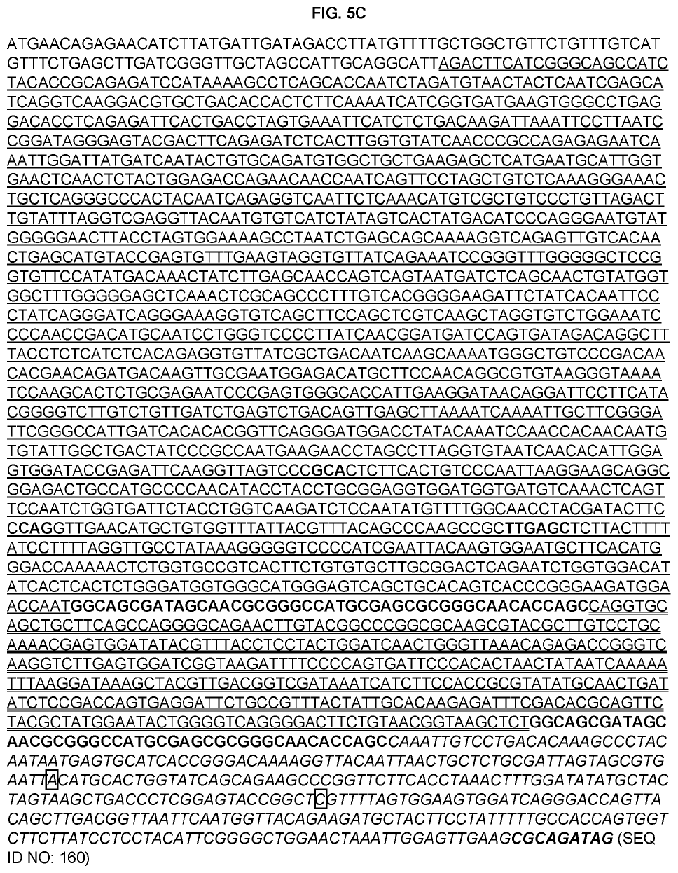

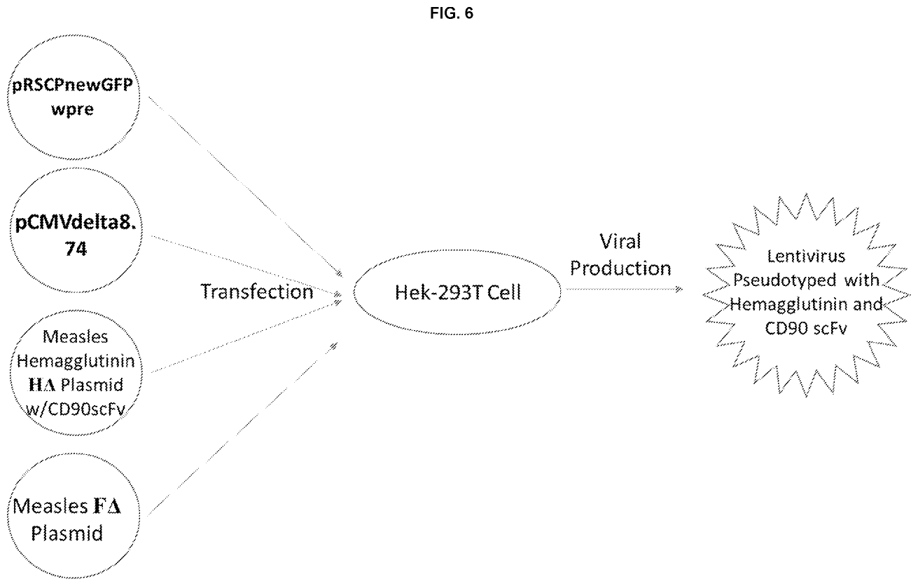

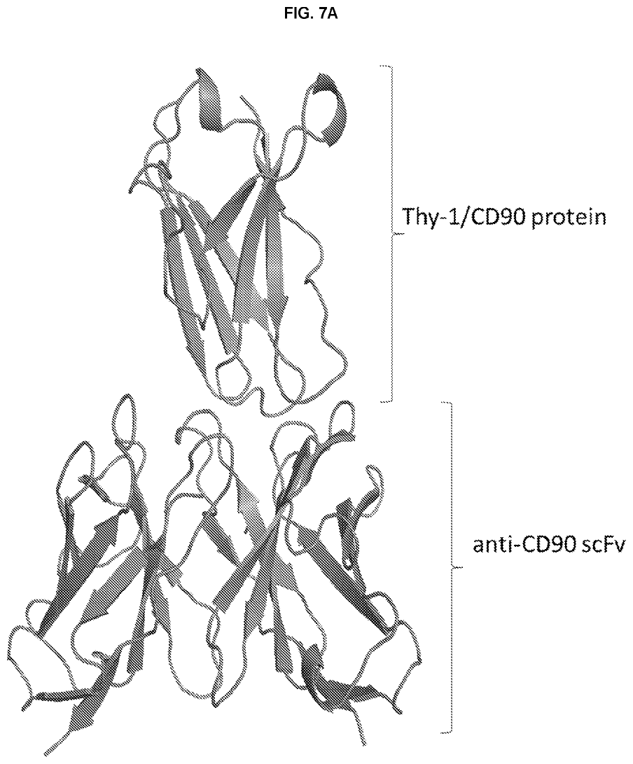

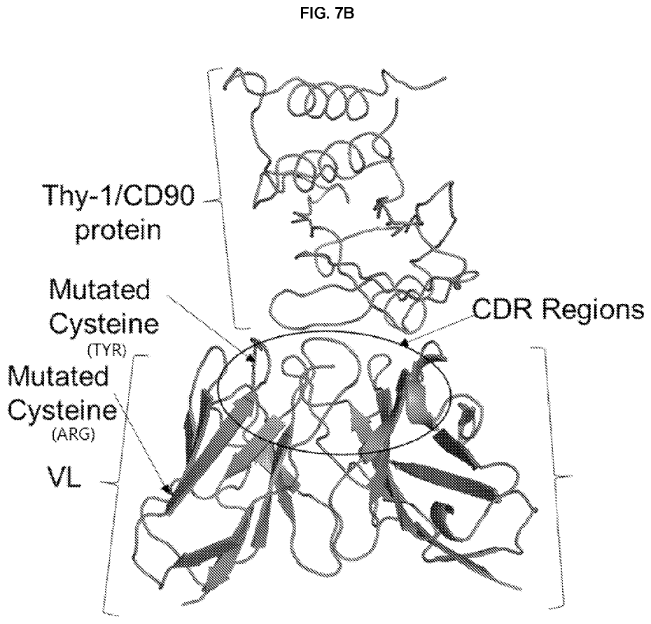

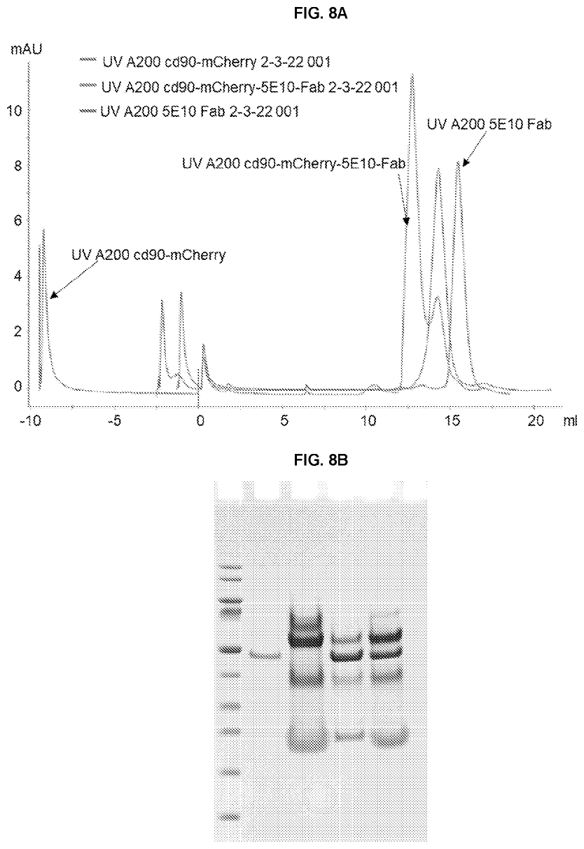

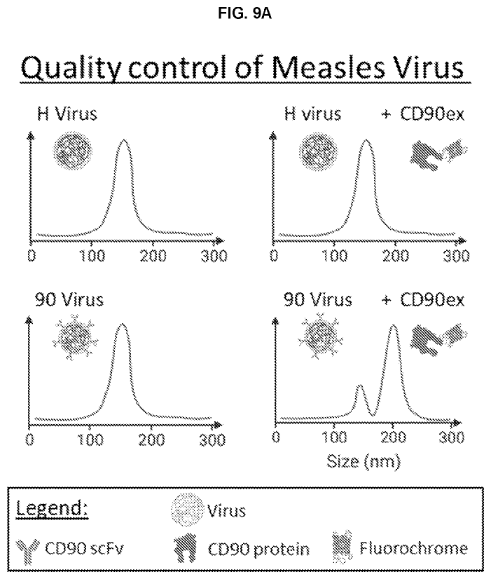

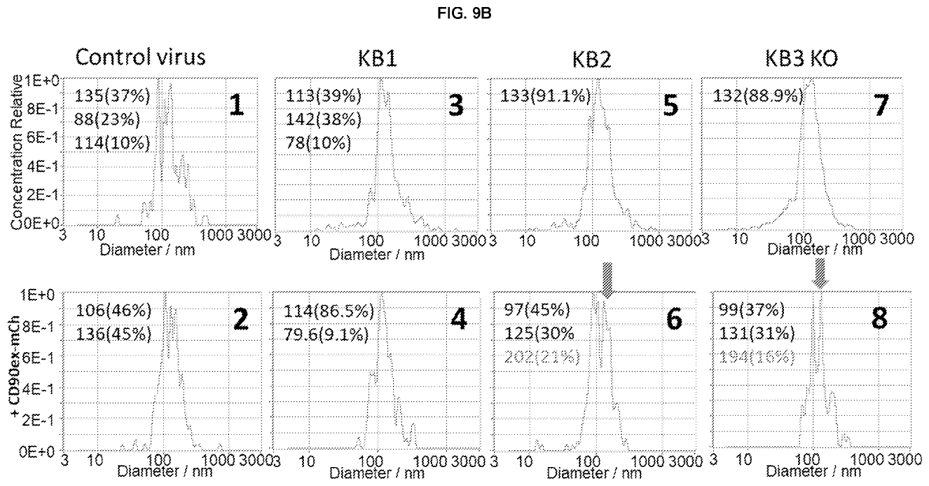

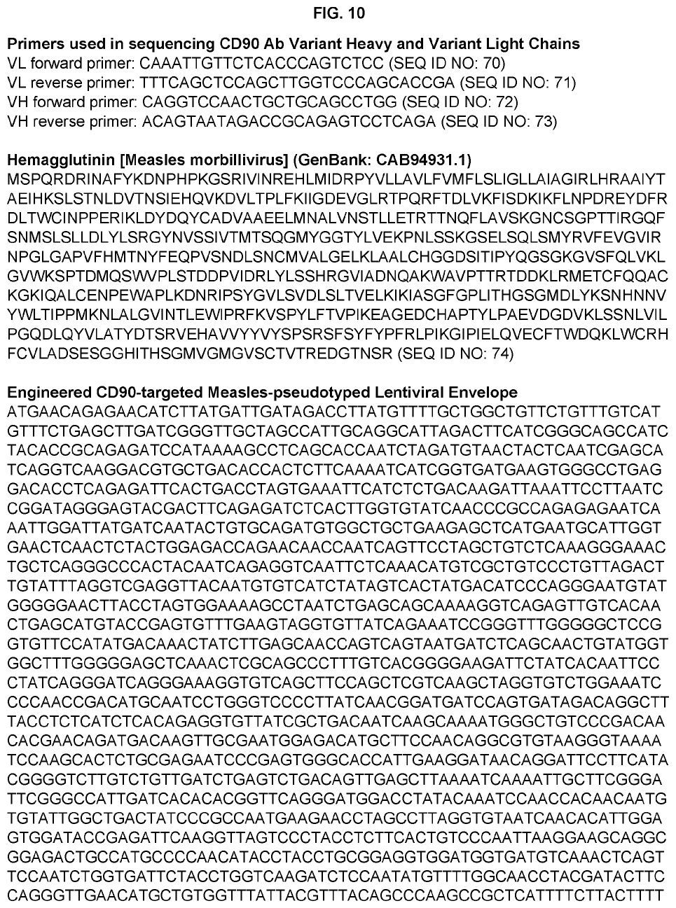

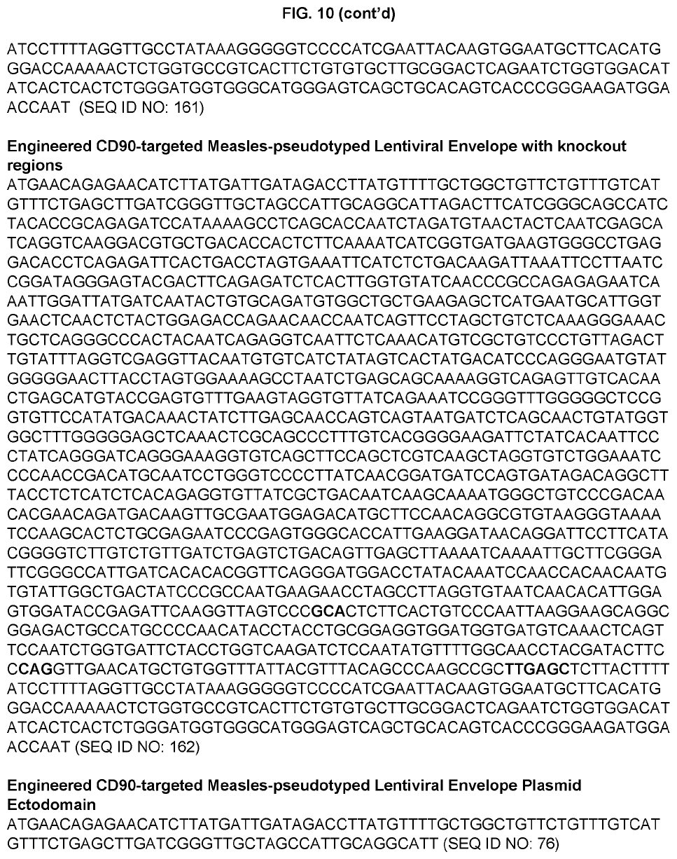

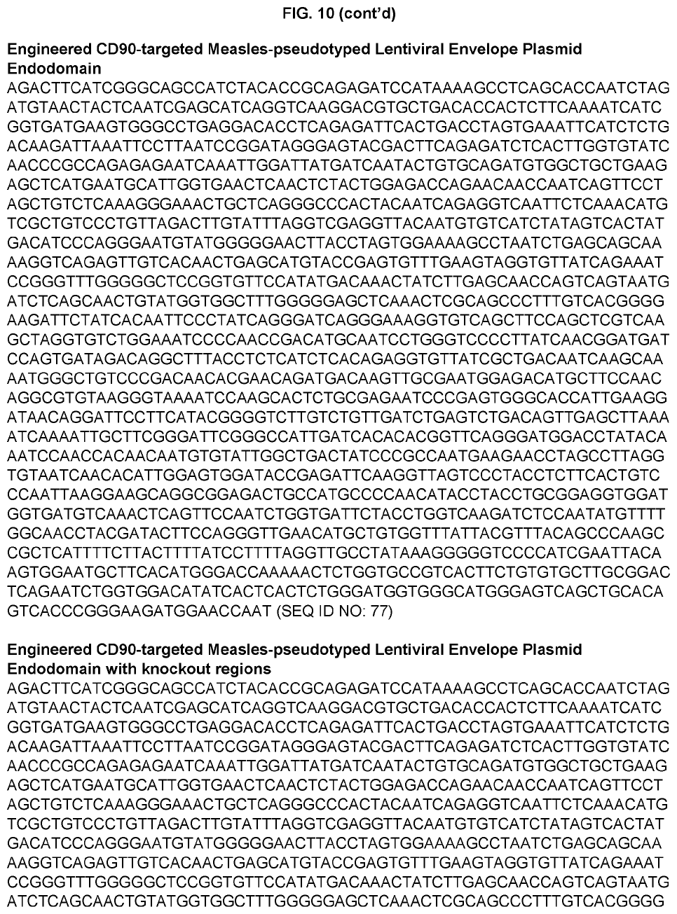

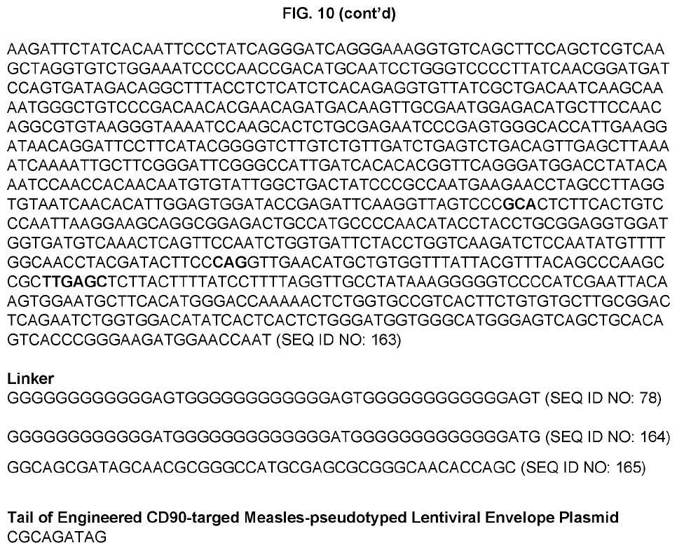

Viral vectors may be further targeted using an antigen-binding portion of an antibody. The current disclosure provides novel scFv sequences that bind CD90 (FIGS. 4A-4C) that can additionally be expressed as a recombinant protein with a structural protein (see, e.g., hemagglutinin, FIGS. 5A and 5B).

In particular embodiments, the anti-CD90 scFv referred to herein as KB1 (SEQ ID NO: 63) is conjugated to a structural protein. In particular embodiments, the anti-CD90 scFv referred to herein as KB2 (SEQ ID NO: 156) is conjugated to a structural protein. In particular embodiments, the anti-CD90 scFv referred to herein as KB3 (SEQ ID NO: 157) is conjugated to a structural protein.

In particular embodiments, the anti-CD90 scFv referred to herein as KB1 (SEQ ID NO: 63) is conjugated to a Measles virus hemagglutinin. In particular embodiments, the anti-CD90 scFv referred to herein as KB2 (SEQ ID NO: 156) is conjugated to a Measles virus hemagglutinin. In particular embodiments, the anti-CD90 scFv referred to herein as KB3 (SEQ ID NO: 157) is conjugated to a Measles virus hemagglutinin.

In particular embodiments, the anti-CD90 scFv referred to herein as KB1 (SEQ ID NO: 63) is conjugated to a VSV-G. In particular embodiments, the anti-CD90 scFv referred to herein as KB2 (SEQ ID NO: 156) is conjugated to a VSV-G. In particular embodiments, the anti-CD90 scFv referred to herein as KB3 (SEQ ID NO: 157) is conjugated to a VSV-G.

The antigen-binding portion of the antibody can be conjugated to the structural protein using any method known to those skilled in the art. Methods of conjugation can include enzymatic conjugation or expressing the recombinant protein as the antigen-binding portion of the antibody linked to the structural protein via a linker. In particular embodiments, the linker includes the sequence as set forth in (GGGGS)₃ (SEQ ID NO: 64). In particular embodiments, the linker includes the sequence as set forth in GSDSNAGHASAGNTS (SEQ ID NO: 66). Additional linkers are described elsewhere herein.

The structural protein can undergo various modifications to improve its production or in vivo characteristics. Modifications can include mutations to evade the immune system and mutations to the cytoplasmic tail to improve viral titer. For example, when the hemagglutinin protein of a measles virus is selected, hemagglutinin from different strains of measles and/or with mutations to mimic different strains can be selected to allow evasion of the human immune system while maintaining transfection. For example, Kneissl et al. (PLoS ONE. 2012, 7(10): e46667) describes a mutated hemagglutinin protein (Hₘᵤₜ) derived from the NSe variant of the measles virus (MV) vaccine strain Edmonston B. Mutations in the MV receptor recognition ectodomain region include Y481A, R533A, S548L and F549S. These modifications in the MV glycoproteins' ectodomain overcame pre-existing MV-specific immunity due to vaccination or natural infection in humans.

Modification in the cytoplasmic tails of measles hemagglutinin can also improve viral titer and can be implemented, for example, as described in Funke et al., (Mol Ther. 2008 August; 16(8):1427-36). Funke et al., describes mutant proteins with cytoplasmic tail truncations of up to 30 residues (leaving, in some instances, only three amino acids).

Additionally, Anliker et al. (Nature Methods. 2010 November; 7(11):929-935) describe modifications to hemagglutinin that abolish measles CD46/SLAM targeting and replaces it with scFvs linked to the end of the hemagglutinin. This approach can further improve cell targeting.

### (iii) Anti-CD90 Antibody Conjugates

An antibody conjugate refers to an antibody or binding fragment thereof disclosed herein linked to another entity. The other entity can be, for example, a toxin, a drug, a detectable label, a radioisotope, a nanoparticle, a bead, or another binding domain. In particular examples, an antibody conjugate is an immunotoxin, an antibody-drug conjugate (ADC), an antibody-detectable label conjugate, an antibody-radioisotope conjugate, an antibody-nanoparticle conjugate, an antibody-bead conjugate, or an antibody with multiple binding domains.

In particular embodiments, antibodies are formed as immunotoxins. Anti-CD90 immunotoxins include an anti-CD90 antibody or binding fragment thereof disclosed herein conjugated to one or more cytotoxins (e.g., protein toxins, enzymatically active toxins of bacterial, fungal, plant, or animal origin, or fragments thereof). A toxin can be any agent that is detrimental to cells. Frequently used plant toxins are divided into two classes: (1) holotoxins (or class II ribosome inactivating proteins), such as ricin, abrin, mistletoe lectin, and modeccin, and (2) hemitoxins (class I ribosome inactivating proteins), such as pokeweed antiviral protein (PAP), saporin, Bryodin 1, bouganin, and gelonin. Commonly used bacterial toxins include diphtheria toxin (DT) and Pseudomonas exotoxin (PE). Kreitman, Current Pharmaceutical Biotechnology 2:313-325 (2001). The toxin may be obtained from essentially any source and can be a synthetic or a natural product.

Immunotoxins with multiple (e.g., four) cytotoxins per binding domain can be prepared by partial reduction of the binding domain with an excess of a reducing reagent such as dithiothreitol (DTT) or tris(2-carboxyethyl)phosphine (TCEP) at 37° C. for 30 min, then the buffer can be exchanged by elution through SEPHADEX G-25 resin with 1 mM DTPA (diethylene triamine penta-acetic acid) in Dulbecco's phosphate-buffered saline (DPBS). The eluent can be diluted with further DPBS, and the thiol concentration of the binding domain can be measured using 5,5′-dithiobis(2-nitrobenzoic acid) [Ellman's reagent]. An excess, for example 5-fold, of a linker-cytotoxin conjugate can be added at 4° C. for 1 hr, and the conjugation reaction can be quenched by addition of a substantial excess, for example 20-fold, of cysteine. The resulting immunotoxin mixture can be purified on SEPHADEX G-25 equilibrated in PBS to remove unreacted linker-cytotoxin conjugate, desalted if desired, and purified by size-exclusion chromatography. The resulting immunotoxin can then be sterile filtered, for example, through a 0.2 μm filter, and can be lyophilized if desired for storage.

In particular embodiments, immunotoxins can include anti-CD90 antibodies or binding fragments thereof conjugated to toxins for targeted CD90+ cell killing.

Antibody-drug conjugates (ADC) allow for the targeted delivery of a drug moiety to a CD90-expressing cell. In particular embodiments, the drug moiety can include a cytotoxic drug or a therapeutic drug or agent.

In particular embodiments, ADC refer to targeted chemotherapeutic molecules which combine properties of both antibodies and cytotoxic drugs by targeting potent cytotoxic drugs to antigen-expressing cancer cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chari, R. V. (2008) Acc. Chem. Res. 41:98-107). See also Kamath & lyer (Pharm Res. 32(11): 3470-3479, 2015), which describes considerations for the development of ADCs.

The cytotoxic drug moiety of the ADC may include any compound, moiety or group that has a cytotoxic or cytostatic effect. Cytotoxic drug moieties may impart their cytotoxic and cytostatic effects by mechanisms including tubulin binding, DNA binding or intercalation, and inhibition of RNA polymerase, protein synthesis, and/or topoisomerase. Exemplary drugs include actinomycin D, anthracycline, auristatin, calicheamicin, camptothecin, CC1065, colchicin, cytochalasin B, daunorubicin, 1-dehydrotestosterone, dihydroxy anthracinedione, dolastatin, doxorubicin, duocarmycin, elinafide, emetine, ethidium bromide, etoposide, gramicidin D, glucocorticoids, lidocaine, maytansinoid (including monomethyl auristatin E [MMAE]; vedotin), mithramycin, mitomycin, mitoxantrone, nemorubicin, PNU-159682, procaine, propranolol, puromycin, pyrrolobenzodiazepine (PBD), taxane, taxol, tenoposide, tetracaine, trichothecene, vinblastine, vinca alkaloid, vincristine, and stereoisomers, isosteres, analogs, and derivatives thereof that have cytotoxic activity.

ADC compounds of the disclosure include those with anti-CD90 activity. In particular embodiments, the ADC compounds include an antibody conjugated, i.e., covalently attached, to the drug moiety. In particular embodiments, the antibody is covalently attached to the drug moiety through a linker. A linker can include any chemical moiety that is capable of linking an antibody, antibody fragment (e.g., antigen binding fragments) or functional equivalent to another moiety, such as a drug moiety. Linkers can be susceptible to cleavage (cleavable linker), such as, acid-induced cleavage, photo-induced cleavage, peptidase-induced cleavage, esterase-induced cleavage, and disulfide bond cleavage, at conditions under which the compound or the antibody remains active. Alternatively, linkers can be substantially resistant to cleavage (e.g., stable linker or noncleavable linker). In some aspects, the linker is a procharged linker, a hydrophilic linker, or a dicarboxylic acid-based linker. The ADCs selectively deliver an effective dose of a drug to cancer cells whereby greater selectivity, i.e. a lower efficacious dose, may be achieved while increasing the therapeutic index (“therapeutic window”).

To prepare ADCs, linker-drug conjugates can be made by conventional methods analogous to those described by Doronina et al. (Bioconjugate Chem. 17: 114-124, 2006). Antibody-drug conjugates with multiple (e.g., four) drugs per antibody can be prepared by partial reduction of the antibody with an excess of a reducing reagent such as dithiothreitol (DTT) or tris(2-carboxyethyl)phosphine (TCEP) at 37° C. for 30 min, then the buffer can be exchanged by elution through SEPHADEX G-25 resin with 1 mM DTPA in Dulbecco's phosphate-buffered saline (DPBS). The eluent can be diluted with further DPBS, and the thiol concentration of the antibody can be measured using 5,5′-dithiobis(2-nitrobenzoic acid) [Ellman's reagent]. An excess, for example 5-fold, of a linker-drug conjugate can be added at 4° C. for 1 hr, and the conjugation reaction can be quenched by addition of a substantial excess, for example 20-fold, of cysteine. The resulting ADC mixture can be purified on SEPHADEX G-25 equilibrated in PBS to remove unreacted linker-drug conjugate, desalted if desired, and purified by size-exclusion chromatography. The resulting ADC can then be sterile filtered, for example, through a 0.2 μm filter, and can be lyophilized if desired for storage.

In particular embodiments, the antibody or binding fragment thereof can be conjugated to a detectable label to form an antibody-detectable label conjugate. A detectable label is a compound or composition that is conjugated directly or indirectly to another molecule, such as an antibody, to facilitate detection of a molecule. Detectable labels can include any suitable label or detectable group detectable by, for example, optical, spectroscopic, photochemical, biochemical, immunochemical, electrical, optical or chemical means. Such detectable labels include fluorescent proteins, enzyme labels, fluorescence labels, radiolabels, radioacoustic labels, chemiluminescence labels, gold beads, magnetic beads (e.g., Dynabeads™), and biotin (with labeled avidin or streptavidin).

Fluorescent proteins can be particularly useful in cell staining, identification, and isolation uses. Exemplary fluorescent proteins include blue fluorescent proteins (e.g. eBFP, eBFP2, Azurite, mKalamal, GFPuv, Sapphire, T-sapphire); cyan fluorescent proteins (e.g. eCFP, Cerulean, CyPet, AmCyanl, Midoriishi-Cyan, mTurquoise); green fluorescent proteins (e.g. GFP, GFP-2, tagGFP, turboGFP, EGFP, Emerald, Azami Green, Monomeric Azami Green (mAzamigreen)), CopGFP, AceGFP, avGFP, ZsGreenl, Oregon Green™ (Thermo Fisher Scientific)); Luciferase; orange fluorescent proteins (mOrange, mKO, Kusabira-Orange, Monomeric Kusabira-Orange, mTangerine, tdTomato); red fluorescent proteins (mKate, mKate2, mPlum, DsRed monomer, mCherry, mRuby, mRFP1, DsRed-Express, DsRed2, DsRed-Monomer, HcRed-Tandem, HcRedl, AsRed2, eqFP611, mRaspberry, mStrawberry, Jred, Texas Red™ (Thermo Fisher Scientific)); far red fluorescent proteins (e.g., mPlum and mNeptune); yellow fluorescent proteins (e.g., YFP, eYFP, Citrine, SYFP2, Venus, YPet, PhiYFP, ZsYellowl); and tandem conjugates.

Exemplary enzyme labels include horseradish peroxidase, hydrolases, and alkaline phosphatase. Exemplary fluorescence labels include rhodamine, phycoerythrin, and fluorescein.

Anti-CD90-radioisotope conjugates (or antibody-radioisotope conjugates) include a CD90 antibody or binding fragment thereof linked to a radioisotope (i.e., radioactive isotope) for use in nuclear medicine. Nuclear medicine refers to the diagnosis and/or treatment of conditions by administering radioactive isotopes (radioisotopes or radionuclides) to a subject. Therapeutic nuclear medicine is often referred to as radiation therapy or radioimmunotherapy (RIT).

Examples of radioactive isotopes that can be conjugated to antibodies or binding fragments thereof of the present disclosure include actinium-225, iodine-131, arsenic-211, iodine-131, indium-111, yttrium-90, and lutetium-177, as well as alpha-emitting radionuclides such as astatine-211 or bismuth-212 or bismuth-213. Methods for preparing radioimmunoconjugates are established in the art. Examples of radioimmunoconjugates are commercially available, including Zevalin™ (DEC Pharmaceuticals), and similar methods can be used to prepare radioimmunoconjugates using the antibodies of the disclosure.

Examples of radionuclides that are useful for radiation therapy include ²²⁵Ac and ²²⁷Th. ²²⁵Ac is a radionuclide with the half-life of ten days. As ²²⁵Ac decays the daughter isotopes ²²¹Fr, ²¹³Bi, and ²⁰⁹Pb are formed. ²²⁷Th has a half-life of 19 days and forms the daughter isotope ²²³Ra.

Additional examples of useful radioisotopes include ²²⁸Ac, ¹¹¹Ag, ¹²⁴Am, ⁷⁴As, ²¹¹As, ²⁰⁹At, ¹⁹⁴Au, ¹²⁸Ba, ⁷Be, ²⁰⁶Bi, ²⁴⁵Bk, ²⁴⁶Bk, ⁷⁶Br, ¹¹C, ⁴⁷Ca, ²⁵⁴Cf, ²⁴²Cm, ⁵¹Cr, ⁶⁷Cu, ¹⁵³Dy, ¹⁵⁷Dy, ¹⁵⁹Dy, ¹⁶⁵Dy, ¹⁶⁶Dy, ¹⁷¹ Er, ²⁵⁰Es, ²⁵⁴Es, ¹⁴⁷Eu, ¹⁵⁷Eu, ⁵²Fe, ⁵⁹Fe, ²⁵¹Fm, ²⁵²Fm, ²⁵³Fm, ⁶⁶Ga, ⁷²Ga, ¹⁴⁶Gd, ¹⁵³Gd ⁶⁸Ge, ¹⁷⁰Hf ¹⁷¹Hf, ¹⁹³Hg, ¹⁹³mHg, ¹⁶⁰mHo, ¹³⁰I, ¹³¹I, ¹³⁵I, ¹¹⁴mIn, ¹⁸⁵Ir, ⁴²K, ⁴³K, ⁷⁶Kr, ⁷⁹Kr, ⁸¹mKr, ¹³²La, ²⁶²Lr, ¹⁶⁹Lu, ¹⁷⁴mLu, ¹⁷⁶mLu, ²⁵⁷Md, ²⁶⁰Md, ²⁸Mg, ⁵²Mn, ⁹⁰Mo, ²⁴Na, ⁹⁵Nb, ¹³⁸Nd, ⁵⁷Ni, ⁶⁶Ni, ²³⁴Np, ¹⁵O, ¹⁸²Os, ¹⁸⁹mOs, ¹⁹¹Os, ³²P, ²⁰¹Pb, ¹⁰¹Pd, ¹⁴³Pr, ¹⁹¹Pt, ²⁴³Pu, ²²⁵Ra, ⁸¹Rb, ¹⁸⁸Re, ¹⁰⁵Rh, ²¹¹Rn, ¹⁰³Ru, ³⁵S, ⁴⁴Sc, ⁷²Se, ¹⁵³Sm, ¹²⁵Sn, ⁹¹Sr, ¹⁷³Ta, ¹⁵⁴Tb, ¹²⁷Te, ²³⁴Th, ⁴⁵Ti, ¹⁶⁶Tm, ²³⁰U, ²³⁷U, ²⁴⁰U, ⁴⁸V, ¹⁷⁸W, ¹⁸¹W, ¹⁸⁸W ¹²⁵Xe, ¹²⁷Xe, ¹³³Xe, ¹³³mXe, ¹³⁵Xe, ⁸⁵mY, ⁸⁶Y, ⁹⁰Y, ⁹³Y, ¹⁶⁹Yb, ¹⁷⁵Yb, ⁶⁵Zn, ⁷¹mZn, ⁸⁶Zr, ⁹⁵Zr, and/or ⁹⁷Zr.

In particular embodiments, the antibody conjugate includes antibody-nanoparticle conjugates. Antibody-nanoparticle conjugates can function in the targeted delivery of a payload (e.g., small molecules or genetic engineering components) to a CD90+ cell ex vivo or in vivo. For example, scFv or other binding fragments can be linked to the surface of nanoparticles to guide delivery to CD90+ cells. The linkage can be through, for example, covalent attachment.

Examples of nanoparticles include metal nanoparticles. Metal nanoparticles can be made of gold, platinum, or silver. Proteins and/or DNA can be conjugated to the surface of metal nanoparticles using surface chemistry. In particular embodiments, the nanoparticle can include gold nanoparticles.

Examples of nanoparticles include liposomes. Liposomes are microscopic vesicles including at least one concentric lipid bilayer. Vesicle-forming lipids are selected to achieve a specified degree of fluidity or rigidity of the final complex. In particular embodiments, liposomes provide a lipid composition that surrounds an aqueous core. In certain examples, the structure of a liposome can be used to encapsulate a nanoparticle within its core (i.e., a liposomal nanoparticle). Lipid nanoparticles (LNPs) are liposome-like structures that lack the continuous lipid bilayer characteristic of liposomes. Solid lipid nanoparticles (SLNs) are LNPs that are solid at room and body temperatures.

Liposomes and similar structures described in the preceding paragraph can be neutral (cholesterol) or bipolar and include phospholipids, such as phosphatidylcholine (PC), phosphatidylethanolamine (PE), phosphatidylinositol (PI), and sphingomyelin (SM) and other type of bipolar lipids including dioleoylphosphatidylethanolamine (DOPE), with a hydrocarbon chain length in the range of 14-22, and saturated or with one or more double C═C bonds. Examples of lipids capable of producing a stable liposome, alone, or in combination with other lipid components are phospholipids, such as hydrogenated soy phosphatidylcholine (HSPC), lecithin, phosphatidylethanolamine, lysolecithin, lysophosphatidylethanolamine, phosphatidylserine, phosphatidylinositol, sphingomyelin, cephalin, cardiolipin, phosphatidic acid, cerebro sides, distearoylphosphatidylethanolamine (DSPE), dioleoylphosphatidylcholine (DOPC), dipalmitoylphosphatidylcholine (DPPC), palmitoyloleoylphosphatidylcholine (POPC), palmitoyloleoylphosphatidylethanolamine (POPE) and dioleoylphosphatidylethanolamine 4-(N-maleimido-methyl)cyclohexane-1-carboxylate (DOPE-mal). Additional non-phosphorous containing lipids that can become incorporated into liposomes include stearylamine, dodecylamine, hexadecylamine, isopropyl myristate, triethanolamine-lauryl sulfate, alkyl-aryl sulfate, acetyl palmitate, glycerol ricinoleate, hexadecyl stereate, amphoteric acrylic polymers, polyethyloxylated fatty acid amides, DDAB, dioctadecyl dimethyl ammonium chloride (DODAC), 1,2-dimyristoyl-3-trimethylammonium propane (DMTAP), DOTAP, DOTMA, DC-Chol, phosphatidic acid (PA), dipalmitoylphosphatidylglycerol (DPPG), dioleoylphosphatidylglycerol, DOPG, and dicetylphosphate. In particular embodiments, lipids used to create liposomes disclosed herein include cholesterol, hydrogenated soy phosphatidylcholine (HSPC) and, the derivatized vesicle-forming lipid PEG-DSPE.

Methods of forming liposomes are described in, for example, U.S. Pat. Nos. 4,229,360; 4,224,179; 4,241,046; 4,737,323; 4,078,052; 4,235,871; 4,501,728; and 4,837,028, as well as in Szoka et al., Ann. Rev. Biophys. Bioeng. 9:467 (1980) and Hope et al., Chem. Phys. Lip. 40:89 (1986). For additional information regarding nanoparticles, see Yetisgin et al., Molecules 2020, 25, 2193.

Nanoparticles can also be formed from polymer and lipid blends. Blends of lipids and polymers in any concentration and in any ratio can also be used. Blending different polymer types in different ratios using various grades can result in characteristics that borrow from each of the contributing polymers. Various terminal group chemistries can also be adopted.

The polymer may be a biodegradable polymer. Examples of polymers that can be used within nanoparticles include polyglutamic acid (PGA); poly(lactic-co-glycolic acid) (PLGA); Polylactic acid (PLA); poly-D-lactic acid (PDLA); PLGA-dimethacrylate; polyamines; polyorganic amines (e.g., polyethyleneimine (PEI), polyethyleneimine celluloses); poly(amidoamines) (PAMAM); polyamino acids (e.g., polylysine (PLL), polyarginine); polysaccharides (e.g, cellulose, dextran, DEAE dextran, starch); spermine, spermidine, poly(vinylbenzyl trialkyl ammonium), poly(4-vinyl-N-alkyl-pyridiumiun), poly(acryloyl-trialkyl ammonium), and Tat proteins. In particular embodiments, the nanoparticle can include a polyglutamic acid (PGA) nanoparticle with a poly(p-amino ester) (PbAE) core. In particular embodiments, nanoparticles are prepared using two steps: 1) genetic engineering components are complexed with a positively-charged PBAE polymer to form nano-sized complexes and 2) an antibody-functionalized PGA is added to the complexes to shield the positive charge and provide targeting (Moffet et al., Nature Communications. 2017. 8(1):389). In particular embodiments, antibody-functionalized PGA includes PGA activated with 1-ethyl-3-(3-dimethylaminopropyl) carbodiimide hydrochloride and combined with antibodies. In particular embodiments, the antibodies include anti-CD90 antibodies. In particular embodiments, the genetic engineering components include mRNA.

In particular embodiments, polymers disclosed herein can include “star shaped polymers,” which refer to branched polymers in which two or more polymer branches extend from a core. The core is a group of atoms having two or more functional groups from which the branches can be extended by polymerization.

Examples of positively charged lipids commonly used in nanoparticles include esters of phosphatidic acid with an aminoalcohol, such as an ester of dipalmitoyl phosphatidic acid or distearoyl phosphatidic acid with hydroxyethylenediamine. More particular examples of positively charged lipids include 3β-[N—(N′,N′-dimethylaminoethyl)carbamoyl) cholesterol (DC-chol); N,N′-dimethyl-N,N′-dioctacyl ammonium bromide (DDAB); N,N′-dimethyl-N,N′-dioctacyl ammonium chloride (DDAC); 1,2-dioleoyloxypropyl-3-dimethyl-hydroxyethyl ammonium chloride (DORI); 1,2-dioleoyloxy-3-[trimethylammonio]-propane (DOTAP); N-(1-(2,3-dioleyloxy)propyl)-N,N,N-trimethylammonium chloride (DOTMA); dipalmitoylphosphatidylcholine (DPPC); 1,2-dioctadecyloxy-3-[trimethylammonio]-propane (DSTAP); and the cationic lipids described in e.g. Martin et al., Current Pharmaceutical Design 2005, 11, 375-394.

Particular embodiments disclosed herein can also utilize porous nanoparticles constructed from any material capable of forming a porous network. Exemplary materials include metals, transition metals and metalloids. Exemplary metals, transition metals and metalloids include lithium, magnesium, zinc, aluminum and silica. In particular embodiments, the porous particles include silica. The exceptionally high surface area of mesoporous silica (exceeding 1,000 m2/g) enables nucleotide loading at levels exceeding conventional DNA carriers such as liposomes.

Nanoparticles can be formed in a variety of different shapes, including spheroidal, cuboidal, pyramidal, oblong, cylindrical, toroidal, and the like. Nanoparticle cargo can be included in the pores of nanoparticles in a variety of ways. For example, nanoparticle cargo can be encapsulated in the porous particles. In other aspects, nanoparticle cargo can be associated (e.g., covalently and/or non-covalently) with the surface or close underlying vicinity of the surface of a porous nanoparticle. In particular embodiments, the nanoparticle cargo can be incorporated in porous nanoparticles e.g., integrated in the material of the porous particles. For example, the nanoparticle cargo can be incorporated into a polymer matrix of polymer nanoparticles.

In particular embodiments, the nanoparticles can include a coating, particularly when used in vivo. A coating can serve to shield the encapsulated cargo and/or reduce or prevent off-target binding. Off-target binding is reduced or prevented by reducing the surface charge of the nanoparticles to neutral or negative. Coatings can include neutral or negatively charged polymer- and/or liposome-based coatings. In particular embodiments, the coating is a dense surface coating of hydrophilic and/or neutrally charged hydrophilic polymer sufficient to prevent the encapsulated cargo from being exposed to the environment before release into a CD90+ cell. In particular embodiments, the coating covers at least 80% or at least 90% of the surface of the nanoparticle. In particular embodiments, the coating includes polyglutamic acid (PGA) or hyaluronic acid. In particular embodiments, PGA can serve as a linker to attach a CD90 binding domain to a nanoparticle.

Examples of neutrally charged polymers that can be used as a nanoparticle coating include polyethylene glycol (PEG); poly(propylene glycol); and polyalkylene oxide copolymers, (PLURONIC®, BASF Corp., Mount Olive, NJ).

Neutrally charged polymers also include zwitterionic polymers. Zwitterionic refers to the property of overall charge neutrality while having both a positive and a negative electrical charge. Zwitterionic polymers can behave like regions of cell membranes that resist cell and protein adhesion.

Zwitterionic polymers include zwitterionic constitutional units including pendant groups (i.e., groups pendant from the polymer backbone) with zwitterionic groups. Exemplary zwitterionic pendant groups include carboxybetaine groups (e.g., —Ra-N+(Rb)(Rc)-Rd-CO₂—, where Ra is a linker group that covalently couples the polymer backbone to the cationic nitrogen center of the carboxybetaine groups, Rb and Rc are nitrogen substituents, and Rd is a linker group that covalently couples the cationic nitrogen center to the carboxy group of the carboxybetaine group).

Examples of negatively charged polymers include alginic acids; carboxylic acid polysaccharides; carboxymethyl cellulose; carboxymethyl cellulose-cysteine; carrageenan (e.g., GELCARIN® 209, GELCARIN® 379, FMC Corporation, Philadelphia, PA); chondroitin sulfate; glycosaminoglycans; mucopolysaccharides; negatively charged polysaccharides (e.g., dextran sulfate); poly(acrylic acid); poly(D-aspartic acid); poly(L-aspartic acid); poly(L-aspartic acid) sodium salt; poly(D-glutamic acid); poly(L-glutamic acid); poly(L-glutamic acid) sodium salt; poly(methacrylic acid); sodium alginate (e.g., PROTANAL® LF 120M, PROTANAL® LF 200M, PROTANAL® LF 200D, FMC Biopolymer Corp., Drammen, Norway); sodium carboxymethyl cellulose (CMC); sulfated polysaccharides (heparins, agaropectins); pectin, gelatin and hyaluronic acid.

The size of particles can vary over a wide range and can be measured in different ways. In preferred embodiments, nanoparticles are <130 nm in size. However, nanoparticles can also have a minimum dimension of equal to or less than 500 nm, less than 150 nm, less than 140 nm, less than 120 nm, less than 110 nm, less than 100 nm, less than 90 nm, less than 80 nm, less than 70 nm, less than 60 nm, less than 50 nm, less than 40 nm, less than 30 nm, less than 20 nm, or less than 10 nm. In particular embodiments, nanoparticles are 90 to 130 nm in size.

Dimensions of the particles can be determined using, e.g., conventional techniques, such as dynamic light scattering and/or electron microscopy.

In particular embodiments, antibody conjugates include antibody-bead conjugates. Antibody-bead conjugates can be prepared by covalently attaching antibodies to a bead. Antibody-bead conjugates can be used for enrichment of CD90+ cells, for example, HSCs or MSCs. The bead can be made of any material suitable for the separation of cells from a cell population. In particular embodiments, the bead can be a magnetic bead or paramagnetic bead.

The sample or composition of cells to be separated is incubated with the antibody-bead conjugates such that the antibody is able to specifically bind to a molecule on the surface of the cell. The cell is then separated from the population of cells by negative or positive selection of the beads. In particular embodiments, the bead is a magnetizable or magnetically responsive material and the cells are selected using a magnetic field. The beads can be Dynabeads or magnetic-activated cell sorting (MACS) (Miltenyi Biotec, Auburn, Calif.). There are many well-known magnetically responsive materials used in magnetic separation methods. Suitable magnetic particles include those described in Molday, U.S. Pat. No. 4,452,773, and in European Patent EP 452342. Colloidal sized particles, such as those described in Owen U.S. Pat. No. 4,795,698, and Liberti et al., U.S. Pat. No. 5,200,084 are other examples.

In particular embodiments, an antibody with multiple binding domains can include binding domains that all bind the same epitope on the same antigen. In particular embodiments, an antibody with multiple binding domains can include binding domains that all bind different epitopes on the same antigen. In particular embodiments, an antibody with multiple binding domains can include binding domains that bind different antigens. A multi-specific antibody has binding domains that bind different epitopes. In particular embodiments, a multi-specific antibody includes a bispecific antibody, a trispecific antibody, or a tetraspecific antibody.

In particular embodiments, anti-CD90 antibody conjugates include antibodies with multiple binding domains wherein at least one binding domain binds CD90. Anti-CD90 bispecific antibodies bind at least two epitopes wherein at least one of the epitopes is located on CD90. Anti-CD90 trispecific antibodies bind at least 3 epitopes, wherein at least one of the epitopes is located on CD90. Anti-CD90 tetraspecific antibodies bind at least 4 epitopes, wherein at least one of the epitopes is located on CD90.

Antibodies with multiple binding domains can be prepared as full-length antibodies or antibody fragments (for example, F(ab′)₂ bispecific antibodies). For example, WO 1996/016673 describes a bispecific anti-ErbB2/anti-Fc gamma RIII antibody; U.S. Pat. No. 5,837,234 describes a bispecific anti-ErbB2/anti-Fc gamma RI antibody; WO 1998/002463 describes a bispecific anti-ErbB2/Fc alpha antibody; and U.S. Pat. No. 5,821,337 describes a bispecific anti-ErbB2/anti-CD3 antibody. In particular embodiments, a bispecific antibody can be in the form of a Bispecific T-cell Engaging (BiTE®) antibody.

Some additional exemplary antibodies with two binding domains (e.g., bispecific antibodies) have two heavy chains (each having three heavy chain CDRs, followed by (N-terminal to C-terminal) a CH1 domain, a hinge, a CH2 domain, and a CH3 domain), and two immunoglobulin light chains that confer antigen-binding specificity through association with each heavy chain. However, as indicated, additional architectures are envisioned, including bi-specific antibodies in which the light chain(s) associate with each heavy chain but do not (or minimally) contribute to antigen-binding specificity, or that can bind one or more of the epitopes bound by the heavy chain antigen-binding regions, or that can associate with each heavy chain and enable binding of one or both of the heavy chains to one or both epitopes.

scFv dimers or diabodies may be used, rather than whole antibodies. Diabodies and scFv can be constructed without an Fc region, using only variable domains (usually including the variable domain components from both light and heavy chains of the source antibody), potentially reducing the effects of anti-idiotypic reaction. Other forms of antibodies with two binding domains include the single chain “Janusins” described in Traunecker et al. (Embo Journal, 10, 3655-3659, 1991).

Exemplary bispecific antibodies with extended half-lives are described in, for example, U.S. Pat. No. 8,921,528 and US Patent Publication No. 2014/0308285.

Methods for making antibodies with two binding domains are known in the art. For example, traditional production of full-length bispecific antibodies is based on the co-expression of two immunoglobulin heavy chain-light chain pairs, where the two chains have different specificities (see, for example, Millstein et al. Nature 305:37-39, 1983). Similar procedures are disclosed in, for example, WO 1993/008829, Traunecker et al., EMBO J. 10:3655-3659, 1991 and Holliger & Winter, Current Opinion Biotechnol. 4, 446-449 (1993).

In particular embodiments, antibodies with two binding domains can be prepared using chemical linkage. For example, Brennan et al. (Science 229: 81, 1985) describes a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated then are converted to thionitrobenzoate (TNB) derivatives. One of the Fab′-TNB derivatives then is reconverted to the Fab′-thiol by reduction with mercaptoethylamine and is mixed with an equimolar amount of the other Fab′-TNB derivative to form the antibody having two binding domains.

Examples of linkers can be found in Chen et al., Adv Drug Deliv Rev. 2013 Oct. 15; 65(10): 1357-1369. Linkers can be flexible, rigid, or semi-rigid, depending on the desired functional domain presentation to a target. Commonly used flexible linkers include linker sequences described elsewhere herein. Linkers that include one or more antibody hinge regions and/or immunoglobulin heavy chain constant regions, such as CH3 alone or a CH2CH3 sequence can also be used.

In some situations, flexible linkers may be incapable of maintaining a distance or positioning of binding domains needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

Anti-CD90 antibodies having three binding domains are artificial proteins that simultaneously bind to three antigens wherein at least one of the antigens is CD90. Anti-CD90 tri-specific antibodies are artificial proteins that simultaneously bind to three different types of antigens, wherein at least one of the antigens is CD90. Tri-specific antibodies are described in, for example, WO2016/105450, WO 2010/028796; WO 2009/007124; WO 2002/083738; US 2002/0051780; and WO 2000/018806.

In particular embodiments, binding domains disclosed herein can be used to create bi- tri, (or more) specific immune cell engaging antibody constructs. An example of a multi-specific immune cell engaging antibody construct includes those which bind both CD90 and an immune cell (e.g., T-cell or NK-cells) activating epitope, with the goal of bringing immune cells to CD90-expressing cells to destroy the CD90-expressing cells. See, for example, US 2008/0145362. Such constructs are referred to herein as immune-activating multi-specifics or I-AMS). BiTEs® (Amgen, Thousand Oaks, CA) are one form of I-AMS. Immune cells that can be targeted for localized activation by I-AMS within the current disclosure include, for example, T-cells, natural killer (NK) cells, and macrophages which are discussed in more detail herein.

T-cell activation can be mediated by two distinct signals: those that initiate antigen-dependent primary activation and provide a T-cell receptor like signal (primary cytoplasmic signaling sequences) and those that act in an antigen-independent manner to provide a secondary or co-stimulatory signal (secondary cytoplasmic signaling sequences). I-AMS disclosed herein can target any T-cell activating epitope that upon binding induces T-cell activation. Examples of such T-cell activating epitopes are on T-cell markers including CD2, CD3, CD7, CD27, CD28, CD30, CD40, CD83, 4-1 BB (CD 137), OX40, lymphocyte function-associated antigen-1 (LFΔ-1), LIGHT, NKG2C, and B7-H3.

In particular embodiments, the CD3 binding domain (e.g., scFv) is derived from the OKT3 antibody (the same as the one utilized in blinatumomab). The OKT3 antibody is described in detail in U.S. Pat. No. 5,929,212. It includes a variable light chain including a CDRL1 sequence including SASSSVSYMN (SEQ ID NO: 92), a CDRL2 sequence including RWIYDTSKLAS (SEQ ID NO: 93), and a CDRL3 sequence including QQWSSNPFT (SEQ ID NO: 94). In particular embodiments, the CD3 T-cell activating epitope binding domain is a human or humanized binding domain (e.g., scFv) including a variable heavy chain including a CDRH1 sequence including KASGYTFTRYTMH (SEQ ID NO: 95), a CDRH2 sequence including INPSRGYTNYNQKFKD (SEQ ID NO: 96), and a CDRH3 sequence including YYDDHYCLDY (SEQ ID NO: 97).

The following sequence is an scFv derived from OKT3 which retains the capacity to bind CD3:

(SEQ ID NO: 98)
QVQLQQSGAELARPGASVKMSCKASGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSSSGGGGSGGGGSGGGGSQIVLTQSPAIMS
ASPGEKVTMTCSASSSVSYMNWYQQKSGTSPKRWIYDTSKLASGVPAHF
RGSGSGTSYSLTISGMEAEDAATYYCQQWSSNPFTFGSGTKLEINR.

It may also be used as a CD3 binding domain.

In particular embodiments, the CD3 T-cell activating epitope binding domain is a human or humanized binding domain (e.g., scFv) including a variable light chain including a CDRL1 sequence including QSLVHNNGNTY (SEQ ID NO: 99), a CDRL2 sequence including KVS, and a CDRL3 sequence including GQGTQYPFT (SEQ ID NO: 100). In particular embodiments, the CD3 T-cell activating epitope binding domain is a human or humanized binding domain (e.g., scFv) including a variable heavy chain including a CDRH1 sequence including GFTFTKAW (SEQ ID NO: 101), a CDRH2 sequence including IKDKSNSYAT (SEQ ID NO: 102), and a CDRH3 sequence including RGVYYALSPFDY (SEQ ID NO:103). These reflect CDR sequences of the 20G6-F3 antibody.

In particular embodiments, the CD3 T-cell activating epitope binding domain is a human or humanized binding domain (e.g., scFv) including a variable light chain including a CDRL1 sequence including QSLVHDNGNTY (SEQ ID NO: 104), a CDRL2 sequence including KVS, and a CDRL3 sequence including GQGTQYPFT (SEQ ID NO: 100). In particular embodiments, the CD3 T-cell activating epitope binding domain is a human or humanized binding domain (e.g., scFv) including a variable heavy chain including a CDRH1 sequence including GFTFSNAW (SEQ ID NO: 105), a CDRH2 sequence including IKARSNNYAT (SEQ ID NO: 106), and a CDRH3 sequence including RGTYYASKPFDY (SEQ ID NO: 107). These reflect CDR sequences of the 4B4-D7 antibody.

In particular embodiments, the CD3 T-cell activating epitope binding domain is a human or humanized binding domain (e.g., scFv) including a variable light chain including a CDRL1 sequence including QSLEHNNGNTY (SEQ ID NO: 108), a CDRL2 sequence including KVS; not included in Sequence Listing), and a CDRL3 sequence including GQGTQYPFT (SEQ ID NO: 100). In particular embodiments, the CD3 T-cell activating epitope binding domain is a human or humanized binding domain (e.g., scFv) including a variable heavy chain including a CDRH1 sequence including GFTFSNAW (SEQ ID NO: 105), a CDRH2 sequence including IKDKSNNYAT (SEQ ID NO: 109), and a CDRH3 sequence including RYVHYGIGYAMDA (SEQ ID NO: 110). These reflect CDR sequences of the 4E7-C9 antibody.

In particular embodiments, the CD3 T-cell activating epitope binding domain is a human or humanized binding domain (e.g., scFv) including a variable light chain including a CDRL1 sequence including QSLVHTNGNTY (SEQ ID NO: 110), a CDRL2 sequence including KVS, and a CDRL3 sequence including GQGTHYPFT (SEQ ID NO: 111). In particular embodiments, the CD3 T-cell activating epitope binding domain is a human or humanized binding domain (e.g., scFv) including a variable heavy chain including a CDRH1 sequence including GFTFTNAW (SEQ ID NO: 112), a CDRH2 sequence including KDKSNNYAT (SEQ ID NO: 113), and a CDRH3 sequence including RYVHYRFAYALDA (SEQ ID NO: 114). These reflect CDR sequences of the 18F5-H10 antibody.

Additional examples of anti-CD3 antibodies, binding domains, and CDRs can be found in WO2016/116626. TR66 may also be used.

CD28 is a surface glycoprotein present on 80% of peripheral T-cells in humans and is present on both resting and activated T-cells. CD28 binds to B7-1 (CD80) and B7-2 (CD86) and is the most potent of the known co-stimulatory molecules (June et al., Immunol. Today 15:321, 1994; Linsley et al., Ann. Rev. Immunol. 11:191, 1993). In particular embodiments, the CD28 binding domain (e.g., scFv) is derived from CD80, CD86 or the 9D7 antibody. Additional antibodies that bind CD28 include 9.3, KOLT-2, 15E8, 248.23.2, and EX5.3D10. Further, 1YJD provides a crystal structure of human CD28 in complex with the Fab fragment of a mitogenic antibody (5.11A1).

In particular embodiments, a CD28 binding domain is derived from TGN1412. In particular embodiments, the variable heavy chain of TGN1412 includes: QVQLVQSGAEVKKPGASVKVSCKASGYTFTSYYIHWVRQAPGQGLEWIGCIYPGNVNTNYNE KFKDRATLTVDTSISTAYMELSRLRSDDTAVYFCTRSHYGLDWNFDVWGQGTTVTVSS (SEQ ID NO: 115) and the variable light chain of TGN1412 includes:

(SEQ ID NO: 116)
DIQMTQSPSSLSASVGDRVTITCHASQNIYVWLNWYQQKPGKAPKLLIY
KASNLHTGVPSRFSGSGSGTDFTLTISSLQPEDFATYYCQQGQTYPYTF
GGGTKVEIK.

In particular embodiments, the CD28 binding domain includes a variable light chain including a CDRL1 sequence including HASQNIYVWLN (SEQ ID NO: 117), CDRL2 sequence including KASNLHT (SEQ ID NO: 118), and CDRL3 sequence including QQGQTYPYT (SEQ ID NO: 119), a variable heavy chain including a CDRH1 sequence including GYTFTSYYIH (SEQ ID NO: 120), a CDRH2 sequence including CIYPGNVNTNYNEK (SEQ ID NO: 121), and a CDRH3 sequence including SHYGLDWNFDV (SEQ ID NO: 122).

In particular embodiments, the CD28 binding domain including a variable light chain including a CDRL1 sequence including HASQNIYVWLN (SEQ ID NO: 117), a CDRL2 sequence including KASNLHT (SEQ ID NO: 118), and a CDRL3 sequence including QQGQTYPYT (SEQ ID NO: 119) and a variable heavy chain including a CDRH1 sequence including SYYIH (SEQ ID NO: 123), a CDRH2 sequence including CIYPGNVNTNYNEKFKD (SEQ ID NO: 124), and a CDRH3 sequence including SHYGLDWNFDV (SEQ ID NO: 122).

Activated T-cells express 4-1BB (CD137). In particular embodiments, the 4-1BB binding domain includes a variable light chain including a CDRL1 sequence including RASQSVS (SEQ ID NO: 125), a CDRL2 sequence including ASNRAT (SEQ ID NO: 126), and a CDRL3 sequence including QRSNWPPALT (SEQ ID NO: 127) and a variable heavy chain including a CDRH1 sequence including YYWS (SEQ ID NO: 128), a CDRH2 sequence including INH, and a CDRH3 sequence including YGPGNYDWYFDL (SEQ ID NO: 129).

In particular embodiments, the 4-1BB binding domain includes a variable light chain including a CDRL1 sequence including SGDNIGDQYAH (SEQ ID NO: 130), a CDRL2 sequence including QDKNRPS (SEQ ID NO: 131), and a CDRL3 sequence including ATYTGFGSLAV (SEQ ID NO: 132) and a variable heavy chain including a CDRH1 sequence including GYSFSTYWIS (SEQ ID NO: 133), a CDRH2 sequence including KIYPGDSYTNYSPS (SEQ ID NO: 134) and a CDRH3 sequence including GYGIFDY (SEQ ID NO: 135).

Particular embodiments disclosed herein including binding domains that bind epitopes on CD8. In particular embodiments, the CD8 binding domain (e.g., scFv) is derived from the OKT8 antibody. For example, in particular embodiments, the CD8 T-cell activating epitope binding domain is a human or humanized binding domain (e.g., scFv) including a variable light chain including a CDRL1 sequence including RTSRSISQYLA (SEQ ID NO: 136), a CDRL2 sequence including SGSTLQS (SEQ ID NO: 137), and a CDRL3 sequence including QQHNENPLT (SEQ ID NO: 138). In particular embodiments, the CD8 T-cell activating epitope binding domain is a human or humanized binding domain (e.g., scFv) including a variable heavy chain including a CDRH1 sequence including GFNIKD (SEQ ID NO: 139), a CDRH2 sequence including RIDPANDNT (SEQ ID NO: 140), and a CDRH3 sequence including GYGYYVFDH (SEQ ID NO: 141). These reflect CDR sequences of the OKT8 antibody.

In particular embodiments natural killer cells (also known as NK-cells, K-cells, and killer cells) are targeted for localized activation by I-AMS. NK cells can induce apoptosis or cell lysis by releasing granules that disrupt cellular membranes and can secrete cytokines to recruit other immune cells.

Examples of activating proteins expressed on the surface of NK cells include NKG2D, CD8, CD16, KIR2DL4, KIR2DS1, KIR2DS2, KIR3DS1, NKG2C, NKG2E, and several members of the natural cytotoxicity receptor (NCR) family. Examples of NCRs that activate NK cells upon ligand binding include NKp30, NKp44, NKp46, NKp80, and DNAM-1.

Examples of commercially available antibodies that bind to an NK cell receptor and induce and/or enhance activation of NK cells include: 5C6 and 1 D11, which bind and activate NKG2D (available from BioLegend® San Diego, CA); mAb 33, which binds and activates KIR2DL4 (available from BioLegend®); P44-8, which binds and activates NKp44 (available from BioLegend®); SK1, which binds and activates CD8; and 3G8 which binds and activates CD16.

In particular embodiments, the I-AMS can bind to and block an NK cell inhibitory receptor to enhance NK cell activation. Examples of NK cell inhibitory receptors that can be bound and blocked include KIR2DL1, KIR2DL2/3, KIR3DL1, NKG2A, and KLRG1. In particular embodiments, a binding domain that binds and blocks the NK cell inhibitory receptors KIR2DL1 and KIR2DL2/3 includes a variable light chain region of the sequence EIVLTQSPVTLSLSPGERATLSCRASQSVSSYLAWYQQKPGQAPRLLIYDASNRATGIPARFSG SGSGTDFTLTISSLEPEDFAVYYCQQRSNWMYTFGQGTKLEIKRT (SEQ ID NO: 142) and a variable heavy chain region of the sequence QVQLVQSGAEVKKPGSSVKVS CKASGGTFSFYAISWVRQAPGQGLEWMGGFIPIFGAANYAQKFQGRVTITADESTSTAYMELS SLRSDDTAVYYCARIPSGSYYYDYDMDVWGQGTTVTVSS (SEQ ID NO: 143). Additional NK cell activating antibodies are described in WO/2005/0003172 and U.S. Pat. No. 9,415,104.

In particular embodiments macrophages are targeted for localized activation by I-AMS. Macrophages are a type of leukocyte (or white blood cell) that can engulf and digest cells, cellular debris, and/or foreign substances in a process known as phagocytosis.

The I-AMS can be designed to bind to a protein expressed on the surface of macrophages. Examples of activating proteins expressed on the surface of macrophages (and their precursors, monocytes) include CD11 b, CD11c, CD64, CD68, CD119, CD163, CD206, CD209, F4/80, IFGR2 Toll-like receptors (TLRs) 1-9, IL-4Ra, and MARCO. Commercially available antibodies that bind to proteins expressed on the surface of macrophages include M1/70, which binds and activates CD11b (available from BioLegend®); KP1, which binds and activates CD68 (available from ABCAM®, Cambridge, United Kingdom); and ab87099, which binds and activates CD163 (available from ABCAM®).

In particular embodiments, I-AMS can target a pathogen recognition receptor (PRR). PRRs are proteins or protein complexes that recognize a danger signal and activate and/or enhance the innate immune response. Examples of PRRs include the TLR4/MD-2 complex, which recognizes gram negative bacteria; Dectin-1 and Dectin-2, which recognize mannose moieties on fungus and other pathogens; TLR2/TLR6 or TLR2/TLR1 heterodimers, which recognize gram positive bacteria; TLR5, which recognizes flagellin; and TLR9 (CD289), which recognizes CpG motifs in DNA. In particular embodiments, I-AMS can bind and activate TLR4/MD-2, Dectin-1, Dectin-2, TRL2/TLR6, TLR2/TLR1, TLR5, and/or TLR9.

In particular embodiments, I-AMS can target the complement system. The complement system refers to an immune pathway that is induced by antigen-bound antibodies and involves signaling of complement proteins, resulting in immune recognition and clearance of the antibody-coated antigens.

### (iv) Compositions for Administration

Any of the antibodies or binding fragments thereof described herein in any exemplary format or conjugation form can be formulated alone or in combination into compositions for administration to subjects. Salts and/or pro-drugs of the antibodies or binding fragments thereof can also be used.

A pharmaceutically acceptable salt includes any salt that retains the activity of the antibody and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage or by hydrolysis of a biologically labile group.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles. Exemplary carriers include saline, buffered saline, physiological saline, water, Hanks' solution, Ringer's solution, Normosol-R (Abbott Labs), Plasma-Lyte A® (Baxter Laboratories, Inc., Morton Grove, IL), glycerol, ethanol, and combinations thereof.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA (ethylene-diamine-tetra-acetic acid).

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the antibodies or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral, sublingual, and/or subcutaneous administration.

For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas.

Additionally, compositions can be formulated as sustained-release systems utilizing semipermeable matrices of solid polymers including at least one type of antibody or binding fragment thereof.

In particular embodiments, the compositions include antibodies or binding fragments thereof of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

### (v) Ex Vivo Cell Manufacturing & Formulation

Anti-CD90 antibodies and binding fragments thereof in any exemplary format or conjugation form described herein can be used in ex vivo cell manufacturing to isolate CD90+ cells from a sample and/or to target CD90+ cells for genetic engineering component delivery.

HSCs can be harvested from, for example, the bone marrow or from peripheral blood of a subject. Mobilization is a process whereby stem cells are stimulated out of the bone marrow (BM) niche into the peripheral blood (PB), and likely proliferate in the PB. Mobilization allows for a larger frequency of stem cells within the PB minimizing the number of days of apheresis, reaching target number collection of stem cells, and minimizing discomfort to the donor source. Agents that enhance mobilization can either enhance proliferation in the PB, or enhance migration from the BM to PB, or both. Mobilizing agents include cytotoxic drugs, cytokines, and/or small molecules. A historically used regimen is a combination of cyclophosphamide (Cy) plus granulocyte-colony stimulating factor (G-CSF) (Bonig et al., Stem Cells, 2009, 27(4): 836-837). Additional mobilizing agents include alpha4-integrin blockade with anti-functional antibodies and CXCR4 blockade with the small-molecule inhibitor AMD3100. AMD3100 is a bicyclam molecule that specifically and reversibly blocks SDF-1 binding to CXCR4. Another embodiment is the combined regiment of GM-CSF or GCSF with AMD3100. In certain embodiments, C4, a CXC chemokine ligand for the CXCR2 receptor. GRObeta rapidly mobilizes short- and long-term repopulating cells in mice and/or monkeys and synergistically enhances mobilization responses with G-CSF (Pelus et al., 2006). Furthermore, GRObeta can be combined with antagonists of VLA4 to synergistically increase circulating HSPC numbers. In certain embodiments, the CXC4 inhibitor, plerixafor, is used as a single agent for mobilization of HSPCs. Plerixafor is also known commercially under the trade names Mozobil, REvixil, UMK121, AMD 3000, AMD 3100, AMD3000, AMD3100, GZ 316455, GZ316455, JM 3100, JM3100, SDZ SID 791, SDZSID791.

MSC are adult, multipotent non-hematopoietic stem cells that have the ability to differentiate into mesodermal lineage (e.g., osteocytes, adipocytes, and chondrocytes), ectodermal lineage (e.g., neurocytes), and endodermal lineage (e.g., hepatocytes). MSC can be isolated from many fetal and adult tissues. Although first being isolated from bone marrow (Pittenger, et al., Science. 1999; 2 84:143-147), MSC can also be isolated from adipose tissue (Wagner, et al., Exp. Hematol. 2005; 33:1402-1416; and Zhang, et al., Calcif. Tissue Int. 2006; 79:169-178), amniotic fluid (In't Anker, et al., Blood. 2003; 102:1548-1549; Tsai, et al., Hum. Reprod. 2004; 19:1450-1456), amniotic membrane (Cai, et al., J. Biol. Chem. 2010; 285:11227-11234), dental tissues (Huang, et al., J. Dent. Res. 2009; 88:792-806; and Seifrtova, et al., Int. Endod. J. 2012; 45:401-412), placenta and fetal membrane (Raynaud, et al., Stem Cells Int. 2012; 2012:658356), sub-amniotic umbilical cord lining membrane (Kita, et al., Stem Cells Dev. 2010; 19:491-502), salivary gland (Rotter, et al., Stem Cells Dev. 2008; 17:509-518), endometrium (Schuring, et al., Fertil. Steril. 2011; 95:423-426), limb bud (Jiao, et al., Cell Reprogram. 2012; 14:324-333), menstrual blood (Allickson, et al., Open Stem Cell J. 2011; 3:4-10), peripheral blood (Ab Kadir, et al., ScientificWorldJournal. 2012; 2012:843843), skin and foreskin (Bartsch, et al., Stem Cells Dev. 2005; 14:337-348; and Riekstina, et al., Cytotechnology. 2008; 58:153-162), synovial fluid (Morito, et al., Rheumatology. 2008; 47:1137-1143) and Wharton's jelly (Wang, et al., Stem Cells. 2004; 22:330-1337; and Hou, et al., Tissue Eng. Part A. 2009; 15:2325-2334). Cell surface markers expressed by MSC include CD29, CD44, CD73, CD90, CD105. MSC lack expression of CD14, CD34, CD45 and human leucocyte antigen (HLA)-DR (Ullah, et al., Biosci Rep. 2015; 35(2): e00191)

CD90+ cells can be collected and isolated from a sample using any appropriate technique. Appropriate collection and isolation procedures include magnetic separation; fluorescence activated cell sorting (FACS; Radtke, et al., 2020, Molecular Therapy 18:679-691; Williams et al., 1985. J Immunol. 135(2): 1004-1011; Lu et al., 1986. Blood 68(1): 126-133); nanosorting based on fluorophore expression; affinity chromatography; cytotoxic agents joined to a monoclonal antibody or used in conjunction with a monoclonal antibody, e.g., complement and cytotoxins; “panning” with antibody attached to a solid matrix (Broxmeyer et al., 1984. J Clin Invest. 73(4):939-953); selective agglutination using a lectin such as soybean (Reisner et al., 1980. Proc Natl Acad Sci. USA 77(11): 6778-6782); immunomagnetic bead-based sorting or combinations of these techniques, etc. These techniques can also be used to assay for successful engraftment or manipulation of cells in vivo, for example for gene transfer, genetic editing or cell population expansion.

Particular embodiments isolate cells based on the marker profile CD34⁺/CD45RA⁻/CD90⁺; CD34⁺/CD45RA⁻/CD90⁺/CD133⁺; CD34⁺/CD45RA⁻/CD90⁻/CD133⁺; CD34⁺/CD45RA⁻/CD90⁺/CD117⁺; CD34⁺/CD45RA⁻/CD90⁻/CD117⁺; CD34⁺/CD45RA⁺/CD90⁻/CD117⁺; CD34⁺/CD45RA⁻/CD90⁻/CD117⁻; or CD34⁺/CD45RA⁻/CD90⁻/CD117⁺/CD123⁺. Particular embodiments isolate cells based on the marker profile CD34⁺/CD45RA⁻/CD90⁺ and/or do not utilize CD38 or CD49f.

In particular embodiments, anti-CD90 antibodies can be used to isolate CD90+ cells. In particular embodiments, compositions described herein can be used to capture CD90+ cells using affinity chromatography. Affinity chromatography refers generally to chromatographic procedures that rely on the specific affinity between a substance to be isolated and a molecule that it can specifically bind to. This is a receptor-ligand type interaction. For example, in particular embodiments, affinity chromatography can be accomplished using columns or beads or other surfaces coated in antibodies or other relevant binding domains. Column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

In particular embodiments, anti-CD90 antibodies or binding fragments thereof are conjugated to magnetic beads for enrichment or isolation of CD90+ cells. In particular embodiments, enrichment or isolation of CD90+ cells includes column-based, affinity-based, or magnetic selection. In particular embodiments, additional antibodies can be used to further refine the desired cell population.

All collected stem cells and stem cell sources can be screened for undesirable components and discarded, treated, or used according to accepted current standards at the time. These stem cell sources can be steady state/nave or primed with mobilizing or growth factor agents.

Undesirable components can be removed from a sample using, for example, biochemical and/or mechanical methods to remove the undesired components and/or remaining cell populations. Examples include lysis cells using detergents, hetastarch, hetastarch with centrifugation, cell washing, cell washing with density gradient, Ficoll-hypaque, Sepx, Optipress, Filters, and other protocols that have been used both in the manufacture of cell and/or gene therapies for research and therapeutic purposes.

In particular embodiments, cell populations can be isolated and/or analyzed based on flow cytometry.

Once cells have been collected and isolated, expansion can be performed. Expansion can occur in the presence of one or more growth factors, such as: angiopoietin-like proteins (Angptls, e.g., Angptl2, Angptl3, Angpt17, Angpt15, and Mfap4); erythropoietin; fibroblast growth factor-1 (FGF-1); FLT3-ligand (FLT3-L); granulocyte colony stimulating factor (G-CSF); granulocyte-macrophage colony stimulating factor (GM-CSF); insulin growth factor-2 (IFG-2); interleukin-3 (IL-3); interleukin-6 (IL-6); interleukin-7 (IL-7); interleukin-11 (IL-11); stem cell factor (SCF; also known as the c-kit ligand or mast cell growth factor); thrombopoietin (TPO); and analogs thereof (wherein the analogs include any structural variants of the growth factors having the biological activity of the naturally occurring growth factor; see, e.g., WO 2007/1145227 and U.S. Patent Publication No. 2010/0183564). For clarity, growth factor agents can also be used as mobilizing agents. Particular embodiments utilize expansion in stem cell supportive media (e.g. StemSpan) supplemented with either SCF, TPO, and FLT3-L or SCF and IL-3, or other combinations of growth factors.

In particular embodiments, the type, amount and/or concentration of growth factors suitable for expanding cells is the amount or concentration effective to promote proliferation. Cell populations are preferably expanded until a sufficient number of cells are obtained to provide for at least one infusion into a human subject, typically around 10⁴ cells/kg to 10⁹ cells/kg.

The amount or concentration of growth factors suitable for expanding cells depends on the activity of the growth factor preparation, and the species correspondence between the growth factors and HSC/MSC, etc. Generally, when the growth factor(s) and cells are of the same species, the total amount of growth factor in the culture medium ranges from 1 ng/ml to 5 μg/ml, from 5 ng/ml to 1 μg/ml, or from 5 ng/ml to 250 ng/ml. In additional embodiments, the amount of growth factors can be in the range of 5-1000 or 50-100 ng/ml.

Cells can be expanded in a tissue culture dish onto which an extracellular matrix protein such as fibronectin (FN), or a fragment thereof (e.g., CH-296 (Dao et. al., 1998, Blood 92(12):4612-21)) or RetroNectin® (a recombinant human fibronectin fragment; (Clontech Laboratories, Inc., Madison, WI) is bound.

During ex vivo manufacturing cells can be genetically modified. Genetic modification is described in the following section.

Ex vivo manufactured cells can be formulated for delivery. Exemplary carriers include saline, buffered saline, physiological saline, water, Hanks' solution, Ringer's solution, Normosol-R (Abbott Labs), Plasma-Lyte A® (Baxter Laboratories, Inc., Morton Grove, IL), glycerol, ethanol, and combinations thereof.

In particular embodiments, carriers can be supplemented with human serum albumin (HSA) or other human serum components or fetal bovine serum. In particular embodiments, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, formulations can include a local anesthetic such as lidocaine to ease pain at a site of injection.

Therapeutically effective amounts of cells within formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

The formulations disclosed herein can be prepared for administration by, for example, injection, infusion, perfusion, or lavage.

It can be necessary or beneficial to cryopreserve a cell and/or cell-based composition. The terms “frozen/freezing” and “cryopreserved/cryopreserving” can be used interchangeably.

Freezing includes freeze drying. As is understood by one of ordinary skill in the art, the freezing of cells can be destructive (see Mazur, P., 1977, Cryobiology 14:251-272) but there are numerous procedures available to prevent such damage. For example, damage can be avoided by (a) use of a cryoprotective agent, (b) control of the freezing rate, and/or (c) storage at a temperature sufficiently low to minimize degradative reactions. Exemplary cryoprotective agents include dimethyl sulfoxide (DMSO) (Lovelock and Bishop, 1959, Nature 183:1394-1395; Ashwood-Smith, 1961, Nature 190:1204-1205), glycerol, polyvinylpyrrolidine (Rinfret, 1960, Ann. N.Y. Acad. Sci. 85:576), polyethylene glycol (Sloviter and Ravdin, 1962, Nature 196:548), albumin, dextran, sucrose, ethylene glycol, i-erythritol, D-ribitol, D-mannitol (Rowe et al., 1962), D-sorbitol, i-inositol, D-lactose, choline chloride (Bender et al., 1960), amino acids (Phan The Tran and Bender, 1960), methanol, acetamide, glycerol monoacetate (Lovelock, 1954), and inorganic salts (Phan The Tran and Bender, 1960; Phan The Tran and Bender, 1961). In particular embodiments, DMSO can be used. Addition of plasma (e.g., to a concentration of 20-25%) can augment the protective effects of DMSO. After addition of DMSO, cells can be kept at 0° C. until freezing, because DMSO concentrations of 1% can be toxic at temperatures above 4° C.

Further considerations and procedures for the manipulation, cryopreservation, and long-term storage of cells, can be found in the following exemplary references: U.S. Pat. Nos. 4,199,022; 3,753,357; and 4,559,298; Gorin, 1986; Bone-Marrow Conservation, Culture and Transplantation, Proceedings of a Panel, Moscow, Jul. 22-26, 1968, International Atomic Energy Agency, Vienna, pp. 107-186; Livesey and Linner, 1987; Linner et al., 1986; Simione, 1992).

Following cryopreservation, frozen cells can be thawed for use in accordance with methods known to those of ordinary skill in the art. Frozen cells are preferably thawed quickly and chilled immediately upon thawing. In particular embodiments, the vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed on ice.

As is understood by one of ordinary skill in the art, if a cryoprotective agent that is toxic to humans is used, it should be removed prior to therapeutic use. DMSO has no serious toxicity.

### (vi) In Vivo or Ex Vivo Genetic Modification of CD90+ Cells

In certain examples, cells will be genetically modified in vivo or ex vivo. For example, CD90+ cells can be genetically modified using a viral vector or nanoparticle to deliver nucleic acids of interest. In particular embodiments, nanoparticle conjugates include anti-CD90 antibodies or binding fragments thereof conjugated to a nanoparticle encapsulating a payload (e.g., a nucleic acid, protein, or small molecule drug) for targeted delivery of genetic engineering components to a CD90+ cell in vivo or ex vivo.

In particular embodiments, gene modification or gene-modifying by gene transfer can be accomplished using any number of DNA or RNA viral vector based or non-viral vector based gene transfer technologies. As indicated previously, viral-mediated gene transfer can be conducted using lentiviral vectors, retroviral vectors (e.g., foamy viral vectors), adenoviral vectors, or adeno-associated viral vectors. In particular embodiments, viral-mediated gene transfer is conducted using pseudotyped viral vectors. Other nucleic acid gene delivery methods include transposon-mediated delivery, plasmid DNA, nanoparticle delivery, or mRNA delivery using transfection, electroporation or nucleofection.

In certain examples, targeted gene editing systems, such as base editors, clustered regularly interspaced short palindromic repeats (CRISPR), zinc fingers (ZFNs), tal effector nucleases (TALENs), meganucleases or meganuclease-TALEN fusions (MegaTALEs) can be used.

Base editors directly convert one base or base pair into another, enabling the efficient installation of point mutations in non-dividing cells without generating excess undesired editing by-products, such as insertions and deletions (indels). With base editors, point mutations can be inserted into DNA or RNA without making double-stranded breaks. DNA base editors include a catalytically disabled nuclease fused to a nucleobase deaminase enzyme and, in some cases, a DNA glycosylase inhibitor. RNA base editors achieve analogous changes using components that base modify RNA. Any nuclease of the CRISPR system can be disabled and used within a base editing system (e.g., dCas9). The nucleobase deaminase enzyme can include a cytidine deaminase domain, an adenine deaminase domain. Further, particular embodiments utilize a uracil glycosylase inhibitor (UGI) as a glycosylase inhibitor.

In particular embodiments, a deaminase domain (cytidine and/or adenine) is fused to the N-terminus of the catalytically disabled nuclease. In these embodiments, a glycosylase inhibitor (e.g., UGI domain) can be fused to the C-terminus of the catalytically disabled nuclease. When multiple glycosylase inhibitors are used, each can be fused to the C-terminus of the catalytically disabled nuclease. Components of base editors can be fused directly (e.g., by direct covalent bond) or via linkers. For additional information regarding base editors, see Seo & Kim, Nature Medicine, 24, 1493-1495 (2018) and Rees & Liu, Nature Reviews Genetics, 19, pages 770-788 (2018).

CRISPR systems and components thereof are described in for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

For information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933, 113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140 as well as Gaj et al., Nat Methods, 2012, 9(8):805-7; Ramirez et al., Nucl Acids Res, 2012, 40(12):5560-8; Kim et al., Genome Res, 2012, 22(7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

TALENs, are described in, for example, U.S. Pat. Nos. 8,440,431; 8,440,432; 8,450,471; 8,586,363; and 8,697,853; as well as Joung and Sander, Nat Rev Mol Cell Biol, 2013, 14(1):49-55; Beurdeley et al., Nat Commun, 2013, 4: 1762; Scharenberg et al., Curr Gene Ther, 2013, 13(4):291-303; Gaj et al., Nat Methods, 2012, 9(8):805-7; Miller, et al. Nature biotechnology 29, 143-148 (2011); Christian, et al. Genetics 186, 757-761 (2010); Boch, et al. Science 326, 1509-1512 (2009); and Moscou, & Bogdanove, Science 326, 1501 (2009).

In certain examples, CD90+ cells are genetically modified to express a gene encoding a therapeutic molecule (e.g., nucleotide sequence or protein). A “gene” refers to a sequence of nucleotides including coding sequences and regulatory regions such as promoters, enhancers, insulators, and/or post-regulatory elements, such as termination regions. The term further can include all introns and other DNA sequences spliced from the mRNA transcript, along with variants resulting from alternative splice sites. The sequences can also include degenerate codons of a reference sequence or sequences that may be introduced to provide codon preference in a specific organism or cell type.

Exemplary genes include ABCA3, ABCD1, Akt, amyloid beta precursor protein (APP), angiopoietin 1 (Ang1), angiotensin-converting enzyme 2 (ACE2), antibodies to CD4, antibodies to CD5, antibodies to CD7, antibodies to CD52, etc., antibodies to IL1, antibodies to IL2, antibodies to IL6, antibodies to TCR specifically present on autoreactive T cells, antibodies to TNF, arylsulfatase A, Bcl-2, brain derived neurotrophic factor (BDNF), cerebral dopamine neurotrophic factor (CDNF), C1q/tumor necrosis factor-related protein-3 (CTRP3), C90RF72, C-C Motif Chemokine Receptor 1 (CCR1), CCR2, chemokine receptor 2 (CXCR2), CXCR4, CXCR7, ciliary neurotrophic factor (CNTF), CLN3, connexin 43 (Cx43), Csx/Nkx 2.5, CTLA, cystic fibrosis transmembrane conductance regulator (CFTR), Cytochrome b5 reductase 3 (CYB5R3), DRB1*1501/DQB1*0602, Dyskerin Pseudouridine Synthase (DKC1), dystrophin, E2F4, endothelial nitric oxide synthase (eNOS), erythropoietin (EPO), extracellular regulating kinase 1/2, F8 (coagulation factor VIII), F9 (coagulation factor IX), Fanconi anaemia complementation group (FANC) family genes, Fas L, fibroblast growth factor-2 (FGF-2), fibroblast growth factor 4 (FGF4), Follistatin-like 1, forkhead box protein (Foxa2), fused in sarcoma (FUS), GATA1, GATA-4, glial cell line-derived neurotrophic factor (GDNF), globin family genes, granulocyte colony-stimulating factor (G-CSF), HBB, heme oxygenase-1 (HO-1), hepatocyte growth factor (HGF), hepatocyte nuclear factor 4α (HNF4α), hypoxia-inducible factor 1α (HIF-1α), insulin-like growth factor (IGF)-1, Intercellular Adhesion Molecule (ICAM)-1, interferon-beta (IFN-β), integrin α4, interleukin-1 receptor antagonist (IL-1Ra), interleukin 4 (IL4), IL10, IL12, IL13, IL-33, Islet-1, Klotho gene, let-7d, leucine-rich repeat kinase 2 (LRRK2), lipocalin 2 (Len2), Mash1, microRNA-1 (miR-1), miR-16-5p, miR-21, miR-25, miR-30b-3p, miR-34, miR-101-3p, miR-124, mIR-126, miR-133, miR-133b, mIR-181a, miR-199a, miR-199a-3p, miR-211, miR-705, nerve growth factor (NGF), neuregulin 4 (Nrg4), neurotrophin-3 (NT3), NLX2.1, Notch ligand Delta-like-4, Notch1 receptor, nuclear factor erythroid-derived 2-like 2 (Nrf2), orphan receptor tyrosine kinase 2 (ROR2), P53, p130, Parkinson disease 2 (PARK2), PARK7, PARKIN, phox, platelet-derived growth factor (PDGF), presenilin 1 (PSEN1), PSEN2, protein tyrosine phosphatase, non-receptor type 22 (PTPN22), PTEN-induced kinase 1 (PINK1), pyruvate kinase, ribosomal protein genes, secreted Klotho protein (SKL), sirtuin 1, soluble CD40, soluble interleukin 1 receptor II (sIL1 RII), sIL1 RI, soluble TNF alpha receptor I (sTNFRI), sTNFRII, somatostatin 2 (sST2), sonic hedgehog (Shh), superoxide dismutase 1 (SOD1), surfactant protein B (SFTPB), SFTPC, synuclein alpha (SNCA), TAR DNA-binding protein 43 (TDP43), telomerase reverse transcriptase (TERT), telomerase RNA component (TERC), TERF interacting nuclear factor 2 (TINF2), TNF-related apoptosis-inducing ligand (TRAIL), transforming growth factor β1 (TGF-β1), type 2 angiotensin II receptor (AT2R), tyrosine kinase receptor type 3 (TrkC), ubiquilin 2, vascular endothelial growth factor (VEGF), WASP Actin nucleation promoting factor (WAS), Wnt/p-catenin,

### (vii) Methods of Use

The compositions or ex vivo manufactured cell-based formulations disclosed herein can be used for treating subjects (humans, veterinary animals (dogs, cats, reptiles, birds, etc.), livestock (horses, cattle, goats, pigs, chickens, etc.), and research animals (monkeys, rats, mice, fish, etc.). Treating subjects includes delivering therapeutically effective amounts. Therapeutically effective amounts include those that provide effective amounts, prophylactic treatments, and/or therapeutic treatments.

An “effective amount” is the amount of a formulation necessary to result in a desired physiological change in a subject. Effective amounts are often administered for research purposes.

A “prophylactic treatment” includes a treatment administered to a subject who does not display signs or symptoms of a condition to be treated or displays only early signs or symptoms of the condition to be treated such that treatment is administered for the purpose of diminishing, preventing, or decreasing the risk of developing the condition. Thus, a prophylactic treatment functions as a preventative treatment against a condition.

A “therapeutic treatment” includes a treatment administered to a subject who displays symptoms or signs of a condition and is administered to the subject for the purpose of reducing the severity or progression of the condition.

Therapeutically effective amounts can achieve more than one type of effect or treatment.

As one example, a gene can be selected to provide a therapeutically effective response against a condition that, in particular embodiments, is inherited. In particular embodiments, the condition can be Grave's Disease, rheumatoid arthritis, pernicious anemia, Multiple Sclerosis (MS), inflammatory bowel disease, systemic lupus erythematosus (SLE), adenosine deaminase deficiency (ADA-SCID) or severe combined immunodeficiency disease (SCID) stemming from deficiency in gamma chain (X-linked SCID), JAK 3 kinase deficiency, purine nucleoside phsosphorylase deficiency, adenosine deaminase (ADA) deficiency, MHC Class II deficiency or recombinase activating gene (RAG) deficiency, Wiskott-Aldrich syndrome (WAS), chronic granulomatous disease (CGD), Fanconi anemia (FΔ), Battens disease, adrenoleukodystrophy (ALD) or metachromatic leukodystrophy (MLD), muscular dystrophy, pulmonary aveolar proteinosis (PAP), pyruvate kinase deficiency, Shwachmann-Diamond-Blackfan anemia, dyskeratosis congenita, xeroderma pigmentosa, cystic fibrosis, Parkinson's disease, Alzheimer's disease, or amyotrophic lateral sclerosis (Lou Gehrig's disease). In particular embodiments, depending on the condition, the therapeutic gene may be a gene that encodes a protein and/or a gene whose function has been interrupted. Exemplary therapeutic gene and gene products include: soluble CD40; CTLA; Fas L; antibodies to CD4, CD5, CD7, CD52, etc.; antibodies to IL1, IL2, IL6; an antibody to TCR specifically present on autoreactive T cells; IL4; IL10; IL12; IL13; IL1Ra, sIL1RI, sIL1RII; sTNFRI; sTNFRII; antibodies to TNF; P53, PTPN22, and DRB1*1501/DQB1*0602; globin family genes; WAS; phox; FANC family genes; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; and/or C90RF72. Therapeutically effective amounts may provide function to immune and other blood cells and/or microglial cells or may alternatively—depending on the treated condition—inhibit lymphocyte activation, induce apoptosis in lymphocytes, eliminate various subsets of lymphocytes, inhibit T cell activation, eliminate or inhibit autoreactive T cells, inhibit Th-2 or Th-1 lymphocyte activity, antagonize IL1 or TNF, reduce inflammation, induce selective tolerance to an inciting agent, reduce or eliminate an immune-mediated condition; and/or reduce or eliminate a symptom of the immune-mediated condition. Therapeutic effective amounts may also provide functional DNA repair mechanisms; surface protein expression; telomere maintenance; lysosomal function; breakdown of lipids or other proteins such as amyloids; permit ribosomal function; and/or permit development of mature blood cell lineages which would otherwise not develop such as macrophages other white blood cell types.

In particular embodiments, HSCs can be genetically modified to provide a therapeutically effective response against diseases related to red blood cells and clotting. In particular embodiments, a gene can be selected to provide a therapeutically effective response against diseases related to red blood cells and clotting. In particular embodiments, the disease is a hemoglobinopathy like thalassemia, or a sickle cell disease/trait. The therapeutic gene may be, for example, a gene that induces or increases production of hemoglobin; induces or increases production of beta-globin, or alpha-globin; or increases the availability of oxygen to cells in the body. The therapeutic gene may be, for example, HBB or CYB5R3. Exemplary effective treatments may, for example, increase blood cell counts, improve blood cell function, or increase oxygenation of cells in patients. In another particular embodiment, the disease is hemophilia. The therapeutic gene may be, for example, a gene that increases the production of coagulation/clotting factor VIII or coagulation/clotting factor IX, causes the production of normal versions of coagulation factor VIII or coagulation factor IX, a gene that reduces the production of antibodies to coagulation/clotting factor VIII or coagulation/clotting factor IX, or a gene that causes the proper formation of blood clots. Exemplary therapeutic genes include F8 and F9. Exemplary effective treatments may, for example, increase or induce the production of coagulation/clotting factors VIII and IX; improve the functioning of coagulation/clotting factors VIII and IX, or reduce clotting time in subjects.

Other uses with supporting detail are found in PCT/US2016/014378.

MSC are widely used in the field of regenerative medicine and have shown therapeutic potential in diseases such as cancer, cardiovascular diseases, lung diseases, gastrointestinal and liver diseases, kidney diseases, and neurological disorders. Several genetic engineering techniques have been used in recent years with the aim of enhancing the therapeutic potential of MSC in order to improve the outcomes after transplantation, increase survival and proliferation, improve pro-regenerative capacity, and induce expression of desired factors (Damasceno et al., 2020. Front. Cell Dev Biol. 8: 737).

MSC can be used to deliver anticancer immunostimulatory agents, such as chemokines and cytokines, to cancer site. In particular embodiments, MSC can be genetically engineered to increase the expression of anticancer agents including: chemokine receptor 4 (CXCR4) (Hmadcha et al., 2020. Front. Bioeng. Biotechnol. 8:43; and Bobis-Wozowicz et al., 2011. Exp. Hematol. 39, 686-696), CXC receptor 7 (CXCR7) (Liu et al., 2018. Sci. Rep. 8, 1-10), cytokine interferon-beta (IFN-β) (Han et al., 2015. Anticancer. Res. 35, 4749-4756), TNF-related apoptosis-inducing ligand (TRAIL) (Spano et al., 2019. Sci. Rep. 9, 1-14), sirtuin 1 (Yu et al., 2016. 7, 71112-71122), hepatocyte nuclear factor 4α (HNF4α) (Wu et al., 2016. Cancer Biol. Ther. 17, 558-565), and microRNAs such as miR-16-5p (Xu et al., 2019. J. Cell. Physiol. 234, 21380-21394), miR-34 (Wang et al., 2019. Stem Cell Res. 41:101605), miR-101-3p and miR-199a (Xie et al., 2019. Mol. Cell. Biochem. 458, 11-26).

MSC can be used to treat cardiovascular disease because of their ability to promote neovascularization, cytoprotection, cardiac regeneration, modulation of inflammatory and fibrogenic processes, cardiac contractility and cardiac metabolism (Gnecchi and Cervio. 2013. Mesenchymal Stem Cell Therapy, eds L. G. Chase and M. C. Vemuri (Totowa, NJ: Humana Press), 241-270). In particular embodiments, MSC can be used in the treatment of cardiovascular diseases by genetic modification to overexpress factors including vascular endothelial growth factor (VEGF) (Locatelli, et al., 2015. Gene Ther. 22, 449-457), hepatocyte growth factor (HGF) (Zhao et al., 2016. Exp. Cell Res. 344, 30-39), insulin-like growth factor (IGF)-1 (Haider et al., 2008. Circ. Res. 103, 11,1300-1308), C1q/tumor necrosis factor-related protein-3 (CTRP3), CCR1 (Zhang et al., 2019. Stem Cell Res. Ther. 10:74), granulocyte colony-stimulating factor (G-CSF) (Silva et al., 2018. Front. Immunol. 9:1449), Csx/Nkx 2.5, GATA-4 (Gao et al., 2011. Circ. J. 75, 11,2683-2691), Akt (Gnecchi et al., 2009. Stem Cells 27, 971-979), CXCR4 (Huang et al., 2012. Cell Biochem. and Funct. 30, 139-144), IL-10 (Meng et al., 2019. J. Biol. Eng. 13:49), IL-33 (Schmitz et al., 2005. Immunity 23, 479-490), endothelial nitric oxide synthase (eNOS) (Chen L. et al., 2017. Cardiovasc. Drugs. Ther. 31, 9-18), heme oxygenase-1 (HO-1) (Liu and Qian, 2015. Int. J. Clin. Exp. Med. 8, 19867-19873), Follistatin-like 1 (Shen et al., 2019. Stem Cell Res. Ther. 10, 1-14), Islet-1 (Xiang et al., 2018. Stem Cell Res. Ther. 9:51), Notch ligand Delta-like-4, Bcl-2, Cx43 and the overexpression of micro RNAs such as mIR-126 (Huang et al., 2013. Int. J. Mol. Med. 31, 484-492), microRNA-1 (Huang et al., 2013. Cardiology 125, 18-30), mIR-181a (Wei et al., 2019. Life Sci. 232:116632), miR-21 (Zeng et al., 2017. Oncotarget. 8, 29161-29173), miR-133 (Chen Y. et al., 2017. Stem Cell Res. Ther. 8:268), and miR-211 (Hu et al., 2016. Stem Cells 34, 1846-1858).

In particular embodiments, MSC can be used and genetically modified to express different factors to promote a patients' recovery from lung disease. In particular embodiments, these factors include HGF, G-CSF (Guo et al., 2013. Heart Vessels 29, 520-531), secreted Klotho protein (SKL) (Varshney et al., 2016. Hypertension 68, 1255-1263), angiopoietin 1 (Ang1) (Mei et al., 2007. PLoS Med. 4:e269), Notch1 receptor (Cheng et al., 2017. Int. J. Chron. Obstruct. Pulmon. Dis. 12, 3133-3147), Wnt/β-catenin (Cai et al. 2015. Stem Cell Res. Ther. 6:65), IL-10, CXCR4, CXCR7 (Wang et al., 2018. DNA Cell Biol. 37, 53-61; Jerkic et al., 2019. J. Clin. Med. 8:847; Shao et al., 2019. Biomed. Pharmacother. 109,1233-1239; Zhang et al., 2019. Stem Cells Int. 2019:2457082), angiotensin-converting enzyme 2 (ACE2) (He et al., 2015. Cell Transplant. 24, 1699-1715), soluble IL-1 receptor type 1 (sST2) (Martinez-Gonzdlez et al., 2014. Stem Cells Dev. 23, 2352-2363), heme-oxygenase-1 (HO-1) (Chen et al., 2018. J. Cell. Physiol. 234, 7301-7319), type 2 angiotensin II receptor (AT2R) (Xu et al., 2018. Stem Cells Transl. Med. 7, 721-730), orphan receptor tyrosine kinase 2 (ROR2) (Cai et al., 2016. Cell Transplant. 25, 1561-1574), nuclear factor erythroid-derived 2-like 2 (Nrf2) (Zhang et al., 2017. J. Cell. Biochem. 119, 1627-1636), and microRNAs such as miR-30b-3p (Yi et al., 2019. Exp. Cell Res. 383:111454), let-7d (Huleihel et al., 2017. Am. J. Physiol. Lung Cell. Mol. Physiol. 313, L92-L103), and p130 and E2F4 (Zhang et al., 2019. Stem Cell Res. Ther. 10:74).

In particular embodiments, MSC can be used and genetically modified to express different factors in the treatment of gastrointestinal and liver diseases. In particular embodiments, these factors include: HGF (Zhang et al., 2018. Cell Death Dis. 9, 1-12), fibroblast growth factor 4 (FGF4) (Wang et al., 2015. Int. J. Clin. Exp. Med. 8, 12774-12782), IGF-1 (Fiore et al., 2015. Stem Cell. Dev. 24, 791-801), neuregulin 4 (Nrg4) (Wang W. et al., 2019. Exp. Biol. Med. 244, 565-578), interleukin-1 receptor antagonist (IL-1 Ra) (Zheng et al., 2012. PLoS One 7:e41392), IL-10 (Choi et al., 2019. Biomater. Sci. 7, 1078-1087), CXCR2 (Shen et al., 2018. Cell Death Dis. 9, 1-14), Intercellular Adhesion Molecule (ICAM)-1 (Li et al., 2019. Stem Cell Res. Ther. 10, 1-11), HNF4a (Ye et al., 2019), and forkhead box protein (Foxa2) (Chae et al., 2019).

In particular embodiments, MSC can be used and genetically modified to express different factors in the treatment of kidney diseases. In particular embodiments, these factors include: glial cell line-derived neurotrophic factor (GDNF) (Huang et al., 2012. J. Control. Release 162, 464-473; Wang. et al., 2019. Stem Cell Res. 41:101605), IGF-1, erythropoietin (EPO) (Kucic et al., 2008. Am. J. Physiol. Renal Physiol. 295, F488-F496), TGF-31 (Cai et al., 2019. Cytotherapy 21, 535-545), lipocalin 2 (Len2) (Roudkenar et al., 2011. Free Radic. Res. 45, 810-819), CXCR4 (Liu et al., 2013. Am. J. Physiol. Renal Physiol. 305, F1064-F1073), Klotho gene (Kuro-o et al., 1997. Nature 390, 45-51), and miR-199a-3p (Zhu et al., 2019. J. Cell. Physiol. 234, 23736-23749).

In particular embodiments, MSC can be used and genetically modified to express different factors in the treatment of several neurological disorders. In particular embodiments, these factors include brain derived neurotrophic factor (BDNF), Glial cell-derived neurotrophic factor (GDNF), fibroblast growth factor-2 (FGF-2), HGF, IGF-1, nerve growth factor (NGF) and platelet-derived growth factor (PDGF) (Joyce et al., 2010. Regen. Med. 5, 933-946), ciliary neurotrophic factor (CNTF) (Abbaszadeh et al., 2015. Cytotherapy 17, 912-921), sonic hedgehog (Shh) (Jia et al., 2014. Neurosci. Lett. 573, 46-51), brain dopamine factor (CDNF) (Zhao et al., 2014. Mol. Neurobiol. 53, 187-199), neurotrophin-3 (NT3) (Moradian et al., 2017. Mater. Sci. Eng. C Mater. Biol. Appl. 76, 934-943), VEGF (Man et al., 2016. Cell. Mol. Bioeng. 9, 96-106), IL-4 (Payne et al., 2012. Cell Adh. Migr. 6, 179-189), IL-10 (Nakajima et al., 2017. Mol. Ther. Methods Clin. Dev. 6, 102-111), C-C Motif Chemokine Receptor 2 (CCR2) (Huang et al., 2018. Theranostics 8, 5929-5944), PARKIN (Bonilla-Porras et al., 2018. Cytotherapy 20, 45-61), extracellular regulating kinase 1/2 or integrin α4 (Gao et al., 2019. Brain Res. Bull. 149, 42-52), miR-705 (Ji et al., 2017. Mol. Med. Rep. 16, 8323-8328), miR-25 (Huang et al., 2018. Exp. Cell Res. 371, 269-277; Zhao et al., 2018. J. Thorac. Cardiovasc. Surg. 157, 508-517), miR-21, miR-124, or miR-133b (Zou et al., 2014. Neural Regen. Res. 9, 1241-1248; and Xin et al., 2017. Cell Transplant. 26, 243-257; Zhang H. et al., 2018. Front. Neurol. 9:931), hypoxia-inducible factor 1α (HIF-1α) (Yang et al., 2014. Int. J. Mol. Med. 34, 1622-1628), Mash1 (Wang et al., 2013. Brain Res. Bull. 99, 84-94), and tyrosine kinase receptor type 3 (TrkC) (Ding et al., 2015. Sci. Rep. 5, 1-14).

For administration, therapeutically effective amounts (also referred to herein as doses) can be initially estimated based on results from in vitro assays and/or animal model studies. For example, a dose can be formulated in animal models to achieve a circulating concentration range that includes an IC₅₀ as determined in cell culture against a particular target. Such information can be used to more accurately determine useful doses in subjects of interest.

The actual dose amount administered to a particular subject can be determined by a physician, veterinarian or researcher taking into account parameters such as physical and physiological factors including target, body weight, severity of condition, type of disease, previous or concurrent therapeutic interventions, idiopathy of the subject and route of administration.

Useful doses of active ingredients within compositions range from, for example, 0.1 μg/kg-5 mg/kg. Administered ex vivo manufactured cells are generally in a volume of a liter or less, 500 mls or less, 250 mls or less or 100 mls or less. Hence the density of administered cells is typically greater than 10⁴ cells/ml, 10⁷ cells/ml, or 10⁸ cells/ml.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly. In particular embodiments, therapeutically effective amounts can be achieved by administering repeated doses during the course of a treatment regimen.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (viii) Exemplary Embodiments

1. A modified antibody or antigen binding fragment thereof including
a variable heavy chain complementarity determining region (CDRH) 1 as set forth in SEQ ID NO: 41, a CDRH2 as set forth in SEQ ID NO: 42, and a CDRH3 as set forth in SEQ ID NO: 43, and
a variable light chain complementarity determining region (CDRL) 1 as set forth in SEQ ID NO: 171, a CDRL2 having the sequence ATS, and a CDRL3 as set forth in SEQ ID NO: 54 according to IMGT;
a CDRH1 as set forth in SEQ ID NO: 44, a CDRH2 as set forth in SEQ ID NO: 45, and a CDRH3 as set forth in SEQ ID NO: 46, and
a CDRL1 as set forth in SEQ ID NO: 173, a CDRL2 as set forth in SEQ ID NO: 56, and a CDRL3 as set forth in SEQ ID NO: 54 according to Kabat;
a CDRH1 as set forth in SEQ ID NO: 47, a CDRH2 as set forth in SEQ ID NO: 48, and a CDRH3 as set forth in SEQ ID NO: 46, and
a CDRL1 as set forth in SEQ ID NO: 173, a CDRL2 as set forth in SEQ ID NO: 56, and a CDRL3 as set forth in SEQ ID NO: 54 according to Chothia; or
a CDRH1 as set forth in SEQ ID NO: 49, a CDRH2 as set forth in SEQ ID NO: 50, and a CDRH3 as set forth in SEQ ID NO: 43, and
a CDRL1 as set forth in SEQ ID NO: 174, a CDRL2 as set forth in SEQ ID NO: 58, and a CDRL3 as set forth in SEQ ID NO: 54 according to North.

2. The modified antibody or antigen binding fragment of embodiment 1, wherein the variable heavy chain has the sequence set forth in SEQ ID NO: 40.

53. The modified antibody or antigen binding fragment of embodiments 1 or 2, wherein the variable light chain has the sequence set forth in SEQ ID NO: 145.

4. An antigen binding fragment of any of embodiments 1-3 including a single chain variable fragment (scFv).

5. The scFv of embodiment 4, wherein the heavy chain has the sequence as set forth in SEQ ID NO: 40.

6. The scFv of embodiments 4 or 5, wherein the light chain includes the sequence as set forth in SEQ ID NO: 145.

7. The scFv of any of embodiments 4-6, wherein the linker sequence has the sequence as set forth in SEQ ID NO: 64, SEQ ID NO: 66, SEQ ID NO: 79, SEQ ID NO: 80, SEQ ID NO: 81, SEQ ID NO: 82. SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, or SEQ ID NO: 91.

8. The scFv of any of embodiments 4-7, wherein the linker sequence has the sequence as set forth in SEQ ID NO: 66.

9. The scFv of any of embodiments 4-8, wherein the linker sequence has the sequence as set forth in SEQ ID NO: 64.

10. The scFv of any of embodiments 4-10, having the sequence as set forth in SEQ ID NO: 156.

11. The scFv of any of embodiments 4-10, having the sequence as set forth in SEQ ID NO: 157.

12. The scFv of any of embodiments 4-11, linked to a viral structural protein.

13. The scFv of embodiment 12, wherein the viral structural protein includes measles virus hemagglutinin (H), measles virus fusion (F), vesicular stomatis virus G (VSV-G), influenza hemagglutinin, ebolavirus glycoprotein (GP)1 or GP2, coronavirus spike, lassa virus glycoprotein precursor (GP) or spike, nipah virus NiV-G or NiV-F, rabies virus glycoprotein (G), respiratory syncytial virus fusion protein (F) or attachment protein (G), human immunodeficiency virus (HIV) gp41, HIV gp120, paramyxovirus F protein, filovirus GP2 protein, flavivirus E protein, alphavirus E1 protein, herpes simplex virus gH or gB protein, or arenavirus G2 protein.

14. The scFv of any of embodiments 12 or 13, wherein the viral structural protein includes measles virus hemagglutinin.

15. The scFv of embodiment 14, wherein the measles virus hemagglutinin includes a cytoplasmic tail truncation of up to 30 residues.

16. The scFv of any of embodiments 14 or 15, wherein the measles virus hemagglutinin includes mutations Y481A, R533A, S548L, and F549S.

17. The scFv of any of embodiments 12-16, encoded by the sequence as set forth in SEQ ID NO: 158.

18. The scFv of any of embodiments 12-17, encoded by the sequence as set forth in SEQ ID NO: 159.

19. The scFv of any of embodiments 12-18, encoded by the sequence as set forth in SEQ ID NO: 160.

20. The modified antibody or antigen binding fragment of any of embodiments 1-19, including mutations in the Fc region.

21. The modified antibody or antigen binding fragment of any of embodiments 1-20, wherein the modified antibody or binding fragment thereof is humanized.

22. The modified antibody or antigen binding fragment of any of embodiments 1-21, wherein the modified antibody or binding fragment thereof is PEGylated.

23. The modified antibody or antigen binding fragment of any of embodiments 1-22, wherein the modified antibody or binding fragment thereof is conjugated to polyglutamic acid (PGA).

24. The modified antibody or antigen binding fragment of any of embodiments 23-24, wherein the PGA shields the positive charge of a polymeric nanoparticle.

25. The modified antibody or antigen binding fragment of any of embodiments 1-24, including M428L/N434S mutations.

26. The modified antibody or antigen binding fragment of any of embodiments 1-25, including G236A/S239D/A330L/1332E mutations.

27. The modified antibody or antigen binding fragment of any of embodiments 1-26, wherein the modified antibody or antigen binding fragment thereof is linked to a viral protein thereby providing a recombinant protein.

28. The modified antibody or antigen binding fragment of embodiment 27, wherein the viral protein includes a structural protein.

29. The modified antibody or antigen binding fragment of embodiment 28, wherein the structural protein includes measles virus hemagglutinin (H), measles virus fusion (F), vesicular stomatis virus G (VSV-G), influenza hemagglutinin, ebolavirus glycoprotein (GP)1 or GP2, coronavirus spike, lassa virus glycoprotein precursor (GP) or spike, nipah virus NiV-G or NiV-F, rabies virus glycoprotein (G), respiratory syncytial virus fusion protein (F) or attachment protein (G), human immunodeficiency virus (HIV) gp41, HIV gp120, paramyxovirus F protein, filovirus GP2 protein, flavivirus E protein, alphavirus E1 protein, herpes simplex virus gH or gB protein, or arenavirus G2 protein.

30. The modified antibody or antigen binding fragment of any of embodiments 1-29, wherein the modified antibody or antigen binding fragment thereof is linked to an toxin, a drug, a detectable label, a radioisotope, a nanoparticle, a bead, or a secondary binding domain.

31. The modified antibody or antigen binding fragment of embodiment 30, wherein the toxin includes a holotoxin or a hemitoxin.

32. The modified antibody or antigen binding fragment of embodiment 30, wherein the drug includes actinomycin D, anthracycline, auristatin, calicheamicin, camptothecin, CC1065, colchicin, cytochalasin B, daunorubicin, 1-dehydrotestosterone, dihydroxy anthracinedione, dolastatin, doxorubicin, duocarmycin, elinafide, emetine, ethidium bromide, etoposide, gramicidin D, glucocorticoids, lidocaine, maytansinoid (including monomethyl auristatin E [MMAE]; vedotin), mithramycin, mitomycin, mitoxantrone, nemorubicin, PNU-159682, procaine, propranolol, puromycin, pyrrolobenzodiazepine (PBD), taxane, taxol, tenoposide, tetracaine, trichothecene, vinblastine, vinca alkaloid, or vincristine.

33. The modified antibody or antigen binding fragment of embodiment 30, wherein the detectable label includes a fluorescent protein, an enzyme label, fluorescent label, or a chemiluminescent label.

34. The modified antibody or antigen binding fragment of embodiment 33, wherein the fluorescent protein includes blue fluorescent protein, cyan fluorescent protein, green fluorescent proteins, luciferase, orange fluorescent protein, red fluorescent protein, far red fluorescent protein, or yellow fluorescent protein.

35. The modified antibody or antigen binding fragment of embodiment 33, wherein the enzyme label includes horseradish peroxidase, hydrolase, or alkaline phosphatase.

36. The modified antibody or antigen binding fragment of embodiment 33, wherein the fluorescent label includes rhodamine, phycoerythrin, or fluorescein.

37. The modified antibody or antigen binding fragment of embodiment 30, wherein the radioisotope includes actinium-225, iodine-131, arsenic-211, iodine-131, indium-111, yttrium-90, lutetium-177, astatine-211, bismuth-212, or bismuth-213.

38. The modified antibody or antigen binding fragment of embodiment 30, wherein the radioisotope includes ²²⁸Ac, ¹¹¹Ag, ¹²⁴Am, ⁷⁴As, ²¹¹As, ²⁰⁹At, ¹⁹⁴Au, ¹²⁸Ba, ⁷Be, ²⁰⁶Bi, ²⁴⁵Bk, ²⁴⁶Bk, ⁷⁶Br, ¹¹C, ⁴⁷Ca, ²⁵⁴Cf, ²⁴²Cm, ⁵¹Cr, ⁶⁷Cu, ¹⁵³Dy, ¹⁵⁷Dy, ¹⁵⁹Dy, ¹⁶⁵Dy, ¹⁶⁶Dy, ¹⁷¹Er, ²⁵⁰Es, ²⁵⁴Es, ¹⁴⁷Eu, ¹⁵⁷Eu, ⁵²Fe, ⁵⁹Fe, ²⁵¹Fm, ²⁵²Fm, ²⁵³Fm, ⁶⁶Ga, ⁷²Ga, ¹⁴⁶Gd, ¹⁵³Gd, ⁶⁸Ge, ¹⁷⁰Hf, ¹⁷¹Hf, ¹⁹³Hg, ¹⁹³mHg, ¹⁶⁰mHo, ¹³⁰I, ¹³¹I, ¹³⁵I, ¹¹⁴mIn, ¹⁸⁵Ir, ⁴²K, ⁴³K, ⁷⁶Kr, ⁷⁹Kr, ⁸¹mKr, ¹³²La, ²⁶²Lr, ¹⁶⁹Lu, ¹⁷⁴mLu, ¹⁷⁶mLu, ²⁵⁷Md, ²⁶⁰Md, ²⁸Mg, ⁵²Mn, ⁹⁰Mo, ²⁴Na, ⁹⁵Nb, ¹³⁸Nd, ⁵⁷Ni, ⁶⁶Ni, ²³⁴Np, ¹⁵O, ¹⁸²Os, ¹⁸⁹mOs, ¹⁹¹Os, ³²P, ²⁰¹Pb, ¹⁰¹Pd, ¹⁴³Pr, ¹⁹¹Pt, ²⁴³Pu, ²²⁵Ra, ⁸¹Rb, ¹⁸⁸Re, ¹⁰⁵Rh, ²¹¹Rn, ¹⁰³Ru, ³⁵S, ⁴⁴Sc, ⁷²Se, ¹⁵³Sm, ¹²⁵Sn, ⁹¹Sr, ¹⁷³Ta, ¹⁵⁴Tb, ¹²⁷Te ²³⁴Th, ⁴⁵Ti, ¹⁶⁶Tm, ²³⁰U, ²³⁷U, ²⁴⁰U, ⁴⁸V, ¹⁷⁸W, ¹⁸¹W, ¹⁸⁸W, ¹²⁵Xe, ¹²⁷Xe, ¹³³Xe, ¹³³mXe, ¹³⁵Xe, ⁸⁵mY, ⁸⁶Y, ⁹⁰Y, ⁹³Y, ¹⁶⁹Yb, ¹⁷⁵Yb, ⁶⁵Zn, ⁷¹mZn, ⁸⁶Zr, ⁹⁵Zr, or ⁹⁷Zr.

39. The modified antibody or antigen binding fragment of embodiment 30, wherein the nanoparticle includes a metal nanoparticle, liposome, a polymer nanoparticle, or a lipid nanoparticle.

40. The modified antibody or antigen binding fragment of embodiment 30, wherein the bead includes a magnetic or a paramagnetic bead.

41. The modified antibody or antigen binding fragment of embodiment 30, wherein the modified antibody or antigen binding fragment thereof and the secondary binding domain form an antibody with multiple binding domains.

42. The modified antibody or antigen binding fragment of embodiment 41, wherein the antibody with multiple binding domains includes binds one epitope, two epitopes, three epitopes, or four epitopes.

43. The modified antibody or antigen binding fragment of any of embodiments 41 or 42, wherein the antibody with multiple binding domains includes a bispecific antibody, a trispecific antibody, or a tetraspecific antibody.

44. The modified antibody or antigen binding fragment of any of embodiments 30-43, wherein the secondary binding domain binds an immune cell activating epitope.

45. The modified antibody or antigen binding fragment of embodiment 44, wherein the secondary binding domain binds a T cell activating epitope or an NK cell activating epitope.

46. The modified antibody or antigen binding fragment of embodiment 45, wherein the T cell activating epitope includes CD3, CD28, 4-1 BB, or CD27.

47. The modified antibody or antigen binding fragment of embodiment 45, wherein the NK cell activating epitope includes CD8, CD16, NKG2A, NKG2D, KIR2DL1, KIR2DL2/3, KIR2DL4, KIR3DL1, NKp44, or KLRG1.

48. A codon-optimized nucleotide sequence encoding the modified antibody or antigen binding fragment of any of embodiments 1-47.

49. The codon-optimized nucleotide sequence of embodiment 48, including a variable heavy chain encoding sequence as set forth in SEQ ID NO: 22, a variable light chain encoding sequence as set forth in SEQ ID NO: 144, and a sequence encoding a linker.

50. The codon-optimized nucleotide sequence of embodiment 49, wherein the sequence encoding the linker includes the sequence as set forth in SEQ ID NO: 78.

51. The codon-optimized nucleotide sequence of any of embodiments 49 or 50, linked to a sequence encoding a viral structural protein with a sequence encoding a second linker.

52. The codon-optimized nucleotide sequence of embodiment 51, wherein the viral structural protein includes measles virus hemagglutinin (H), measles virus fusion (F), vesicular stomatis virus G (VSV-G), influenza hemagglutinin, ebolavirus glycoprotein (GP)1 or GP2, coronavirus spike, lassa virus glycoprotein precursor (GP) or spike, nipah virus NiV-G or NiV-F, rabies virus glycoprotein (G), respiratory syncytial virus fusion protein (F) or attachment protein (G), human immunodeficiency virus (HIV) gp41, HIV gp120, paramyxovirus F protein, filovirus GP2 protein, flavivirus E protein, alphavirus E1 protein, herpes simplex virus gH or gB protein, or arenavirus G2 protein.

53. The codon-optimized nucleotide sequence of any of embodiments 51 or 52, wherein the viral structural protein includes measles virus hemagglutinin.

54. The codon-optimized nucleotide sequence of any of embodiments 51 or 52, wherein the viral structural protein includes VSV-G.

55. The codon-optimized nucleotide sequence of any of embodiments 51-54, wherein the sequence encoding a viral structural protein includes the sequence as set forth in SEQ ID NO: 161 or SEQ ID NO: 162 or a sequence having at least 95% sequence identity to a sequence as set forth in SEQ ID NO: 161 or SEQ ID NO: 162.

56. The codon-optimized nucleotide sequence of any of embodiments 51-55, wherein the sequence encoding the second linker includes the sequence as set forth in SEQ ID NO: 64, SEQ ID NO: 66, SEQ ID NO: 78, SEQ ID NO: 164, or SEQ ID NO: 165 or a sequence having at least 95% sequence identity to a sequence as set forth in SEQ ID NO: 64, SEQ ID NO: 66, SEQ ID NO: 78, SEQ ID NO: 164, or SEQ ID NO: 165.

57. A composition including an antibody or antigen binding fragment of any of embodiments 1-47 and a pharmaceutically acceptable carrier.

58. A pseudotyped viral vector including an antibody or antigen binding fragment of any of embodiments 1-19 linked to a viral structural protein.

59. The pseudotyped viral vector of embodiment 58, wherein the viral structural protein includes measles virus hemagglutinin (H), measles virus fusion (F), vesicular stomatis virus G (VSV-G), influenza hemagglutinin, ebolavirus glycoprotein (GP)1 or GP2, coronavirus spike, lassa virus glycoprotein precursor (GP) or spike, nipah virus NiV-G or NiV-F, rabies virus glycoprotein (G), respiratory syncytial virus fusion protein (F) or attachment protein (G), human immunodeficiency virus (HIV) gp41, HIV gp120, paramyxovirus F protein, filovirus GP2 protein, flavivirus E protein, alphavirus E1 protein, herpes simplex virus gH or gB protein, or arenavirus G2 protein.

60. The pseudotyped viral vector of any of embodiments 58 or 59, wherein the viral structural protein includes a measles virus hemagglutinin.

61. The pseudotyped viral vector of any of embodiments 58 or 59, wherein the viral structural protein includes VSV-G.

62. The pseudotyped viral vector of any of embodiments 58 or 59, wherein the viral vector is a lentiviral vector, a retroviral vector, an adenoviral vector, or adeno-associated viral (AAV) vector.

63. A pseudotyped viral vector expressing a recombinant protein encoded by the codon-optimized nucleotide sequence of any of embodiments 48-56.

64. The pseudotyped viral vector of embodiment 63, wherein the viral vector includes a lentiviral vector, a retroviral vector, or an adenoviral vector.

65. Use of an antibody or antigen binding fragment of any of embodiments 1-47 to target CD90+ cells for delivery of genetic engineering components.

66. The use of embodiment 65 wherein the genetic engineering components provide a therapeutic gene to the CD90+ cell.

67. The use of any of embodiments 65 or 66, wherein the genetic engineering components insert or alter a gene selected from ABCA3, ABCD1, Akt, amyloid beta precursor protein (APP), angiopoietin 1 (Ang1), angiotensin-converting enzyme 2 (ACE2), antibodies to CD4, antibodies to CD5, antibodies to CD7, antibodies to CD52, etc., antibodies to IL1, antibodies to IL2, antibodies to IL6, antibodies to TCR specifically present on autoreactive T cells, antibodies to TNF, arylsulfatase A, Bcl-2, brain derived neurotrophic factor (BDNF), cerebral dopamine neurotrophic factor (CDNF), C1q/tumor necrosis factor-related protein-3 (CTRP3), C90RF72, C-C Motif Chemokine Receptor 1 (CCR1), CCR2, chemokine receptor 2 (CXCR2), CXCR4, CXCR7, ciliary neurotrophic factor (CNTF), CLN3, connexin 43 (Cx43), Csx/Nkx 2.5, CTLA, cystic fibrosis transmembrane conductance regulator (CFTR), Cytochrome b5 reductase 3 (CYB5R3), DRB1*1501/DQB1*0602, Dyskerin Pseudouridine Synthase (DKC1), dystrophin, E2F4, endothelial nitric oxide synthase (eNOS), erythropoietin (EPO), extracellular regulating kinase 1/2, F8 (coagulation factor VIII), F9 (coagulation factor IX), Fanconi anaemia complementation group (FANC) family genes, Fas L, fibroblast growth factor-2 (FGF-2), fibroblast growth factor 4 (FGF4), Follistatin-like 1, forkhead box protein (Foxa2), fused in sarcoma (FUS), GATA1, GATA-4, glial cell line-derived neurotrophic factor (GDNF), globin family genes, granulocyte colony-stimulating factor (G-CSF), HBB, heme oxygenase-1 (HO-1), hepatocyte growth factor (HGF), hepatocyte nuclear factor 4α (HNF4α), hypoxia-inducible factor 1α (HIF-1α), insulin-like growth factor (IGF)-1, Intercellular Adhesion Molecule (ICAM)-1, interferon-beta (IFN-β), integrin α4, interleukin-1 receptor antagonist (IL-1Ra), interleukin 4 (IL4), IL10, IL12, IL13, IL-33, Islet-1, Klotho gene, let-7d, leucine-rich repeat kinase 2 (LRRK2), lipocalin 2 (Len2), Mash1, microRNA-1 (miR-1), miR-16-5p, miR-21, miR-25, miR-30b-3p, miR-34, miR-101-3p, miR-124, mIR-126, miR-133, miR-133b, mIR-181a, miR-199a, miR-199a-3p, miR-211, miR-705, nerve growth factor (NGF), neuregulin 4 (Nrg4), neurotrophin-3 (NT3), NLX2.1, Notch ligand Delta-like-4, Notch1 receptor, nuclear factor erythroid-derived 2-like 2 (Nrf2), orphan receptor tyrosine kinase 2 (ROR2), P53, p130, Parkinson disease 2 (PARK2), PARK7, PARKIN, phox, platelet-derived growth factor (PDGF), presenilin 1 (PSEN1), PSEN2, protein tyrosine phosphatase, non-receptor type 22 (PTPN22), PTEN-induced kinase 1 (PINK1), pyruvate kinase, ribosomal protein genes, secreted Klotho protein (SKL), sirtuin 1, soluble CD40, soluble interleukin 1 receptor II (sIL1 RII), sIL1 RI, soluble TNF alpha receptor I (sTNFRI), sTNFRII, somatostatin 2 (sST2), sonic hedgehog (Shh), superoxide dismutase 1 (SOD1), surfactant protein B (SFTPB), SFTPC, synuclein alpha (SNCA), TAR DNA-binding protein 43 (TDP43), telomerase reverse transcriptase (TERT), telomerase RNA component (TERC), TERF interacting nuclear factor 2 (TINF2), TNF-related apoptosis-inducing ligand (TRAIL), transforming growth factor p31 (TGF-p1), type 2 angiotensin II receptor (AT2R), tyrosine kinase receptor type 3 (TrkC), ubiquilin 2, vascular endothelial growth factor (VEGF), WASP Actin nucleation promoting factor (WAS), or Wnt/β-catenin.

68. The use of any of embodiments 65-67, wherein the CD90+ cells are hematopoietic stem cells or mesenchymal stem/stromal cells.

69. The use of any of embodiments 65-68, wherein the genetic engineering components include naked DNA, naked mRNA, guideRNA, a base editor, a clustered regularly interspaced short palindromic repeats (CRISPR) nuclease, a zinc finger (ZFN), a tal effector nuclease (TALEN), a meganucleases or a meganuclease-TALEN fusion (MegaTALE).

### (ix) Experimental Example

CD90-targeted measles-pseudotyped lentiviral vectors for HSC gene therapy.

Abstract. Hematopoietic stem cell (HSC) gene therapy is currently performed on CD34⁺ hematopoietic stem and progenitor cells (HSPCs) containing less than 1% true HSCs and requiring a highly specialized infrastructure for cell manufacturing and transplantation. The CD34⁺CD90⁺ subset has previously been identified to be exclusively responsible for short- and long-term engraftment. However, purification and enrichment of this subset is laborious and expensive. HSC-specific delivery agents for the direct modification of rare HSCs are currently lacking. Here, novel targeted viral vectors were developed to specifically transduce CD90-expressing HSCs. Anti-CD90 single chain variable fragments (scFvs) were engineered onto measles- and VSV-G-pseudotyped lentiviral vectors that were knocked out for native targeting. A custom hydrodynamic titration methodology was developed to assess the loading of surface-engineered capsids, measure antigen recognition of the scFv, and predict the performance on cells. Engineered vectors formed with minimal impairment in the functional titer, maintained their ability to fuse with the target cells, and showed highly specific recognition of CD90 on cells ex vivo. Most importantly, targeted vectors selectively transduced human HSCs with secondary colony-forming potential. The disclosed HSC-targeted viral vectors significantly enhance the feasibility of ex vivo gene therapy and pave the way for future in vivo applications.

Introduction. Currently existing gene therapy approaches modify CD34⁺ hematopoietic stem and progenitor cells (HSPCs), a heterogeneous population that contains less than 1% HSCs with long-term multilineage engraftment potential (Majeti, et al., 2007, Cell Stem Cell 1, 635-645). Consequently, massive use of expensive reagents, potential off-target effects in non-HSCs, and low on-target efficiency currently impact the feasibility of HSC gene therapy. In addition, current gene therapy approaches rely on the modification of HSCs outside the patient's body (ex vivo) and require highly specialized facilities similar to bone marrow transplantation, severely limiting the accessibility of this treatment option. Injection of gene therapy agents and modification of HSCs directly in the patient (in vivo) would overcome all these limitations. However, a major obstacle to perform gene therapy in vivo is the lack of HSC-targeted gene therapy agents to highly specifically deliver the therapeutic cargo and avoid off-target effects.

This targeted delivery depends on the uptake of the vector after attaching to the cell/antigen, which has not been achieved with CD34-targeted HSC gene therapy vectors (Benedict, et al., 1999, Hum Gene Ther 10, 545-557). As a result, alternative markers of HSCs such as CD117 and CD133 have been considered (Radtke, et al., 2020, Mol Ther Methods Clin Dev 18, 679-691; and Brendel, et al., 2015, Mol Ther 23, 63-70). However, CD117⁺/CD133⁺ cells contain various erythro-myeloid and lympho-myeloid progenitors (Gorgens, et al., 2013, Cell Rep 3, 1539-1552; and Radtke, et al., 2015, Br J Haematol 169, 868-878) that bear the risk for unwanted off-target effects. Further, CD133 as well as CD117 are readily expressed on cells in the lung, liver, and reproductive organs (proteinatlas.org), likely leading to rapid clearance of CD133/CD117-targeted vectors as well as unknown side-effects when applied in vivo.

Previous work has shown that the CD90⁺ subset of CD34⁺ HSPCs is exclusively responsible for rapid recovery onset, robust long-term multilineage engraftment, and an entire reconstitution of the bone marrow (BM) stem cell compartment (Radtke, et al., 2020, Mol Ther Methods Clin Dev 18, 679-691; Radtke, et al., 2017, Sci Transl Med 9. 10.1126/scitranslmed.aan1145; and Humbert, et al., 2019, Sci Transl Med 11, eaaw3768. 10.1126/scitranslmed.aaw3768). Furthermore, CD34⁺CD90⁺ cells are almost entirely depleted for phenotypically, transcriptionally, and functionally committed progenitor cells (Radtke, et al., 2020, Mol Ther Methods Clin Dev 18, 679-691; Masiuk, et al., 2017, Mol Ther 25, 2163-2175; Zonari, et al., 2017, Stem Cell Reports 8, 977-990; and Gordon, et al., 2003, Bone Marrow Transplant 31, 17-22). Isolation and direct targeting of this HSC-enriched subset from mobilized human apheresis products ex vivo significantly increased lentivirus-mediated gene transfer without impacting the cells long-term multilineage engraftment potential (Benedict, et al., 1999, Hum Gene Ther 10, 545-557).

To enable highly-specific targeting of CD90⁺ HSCs, CD90-targeted viral vectors engineering an anti-CD90 single chain variable fragment (scFv) onto measles- and VSV-G-pseudotyped lentiviral vectors were herein designed. CD90-targeted viral vectors were further knocked out for their native targeting to optimize on-target specificity. Lastly, a hydrodynamics-based methodology was developed to easily and reliably titrate and validate the disclosed surface-engineered viral vectors.

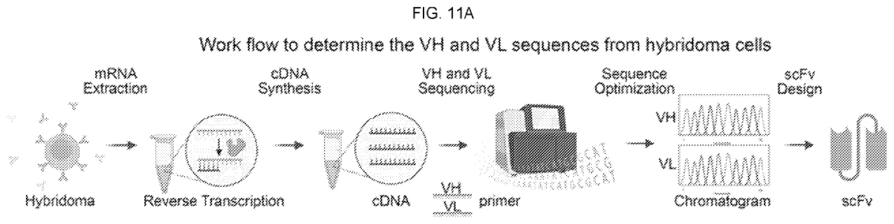

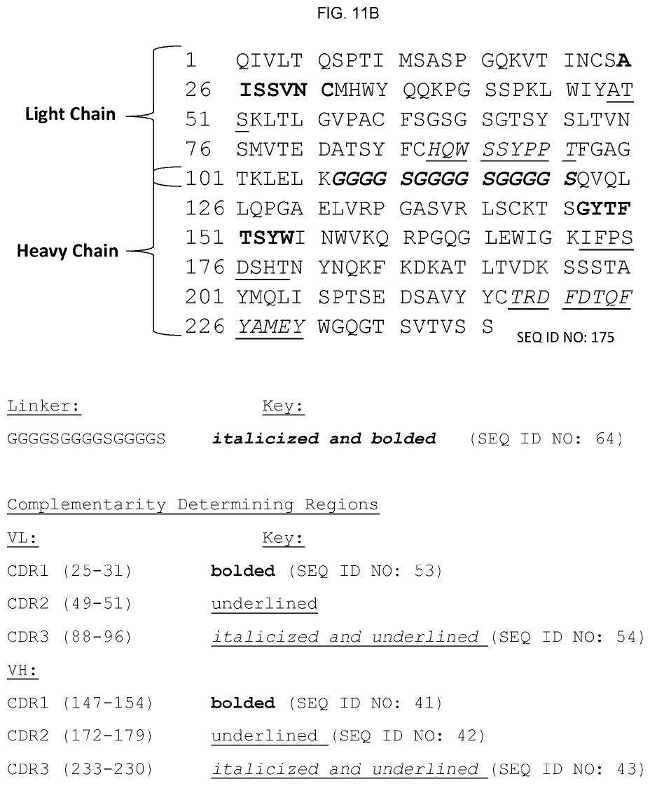

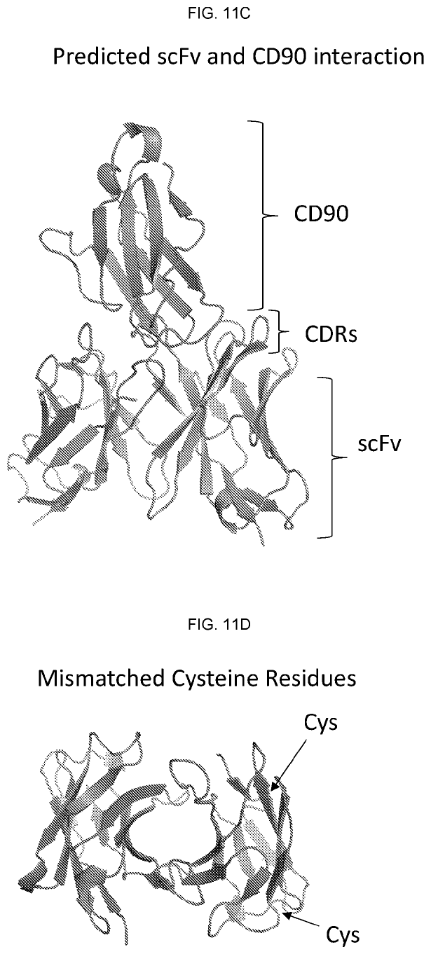

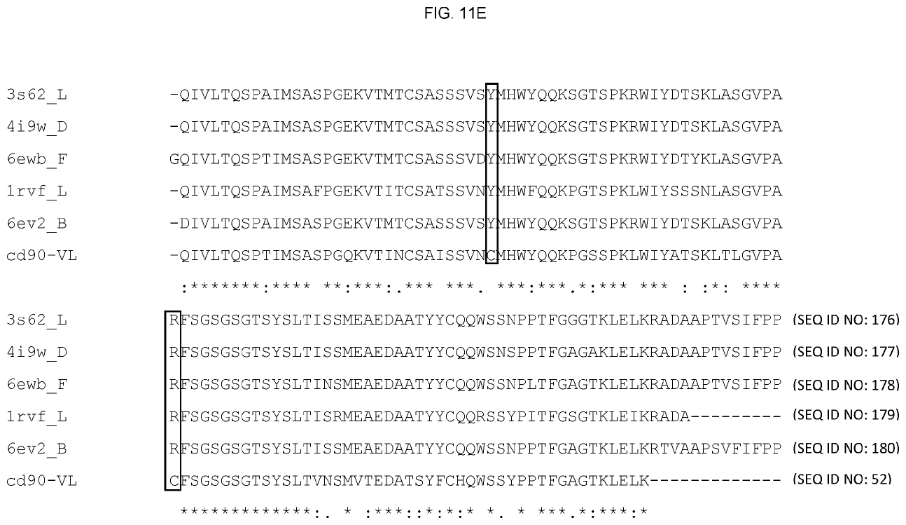

Results. CD90 scFv Design, Modification, and Validation. In order to generate HSC-targeted viral vectors for gene therapy, an scFv recognizing CD90 was designed and computationally validated. Sequences for the scFv design were extracted from the hybridoma cell line producing the CD90 antibody clone 5E10 (Craig, et al., 1993, J Exp Med 177, 1331-1342). Briefly, mRNA was extracted from the cell line, reverse transcribed, and primers designed to sequence the cDNA fragments encoding for the variant light and heavy regions (FIG. 11A). To determine the framework and complementary defining regions of the antibody, sequences were annotated using IMGT (Lefranc, et al., 2015, Nucleic Acids Res 43, D413-D422; and Lefranc, 2014, Front Immunol 5, 22). Next, scFvs were designed linking the light- and heavy-chain sequences using a 3x(GGGGS) (SEQ ID NO: 64) linker (FIG. 11B). Finally, to predict the binding ability of the anti-CD90 scFv with the extracellular domain of CD90, interactions were modeled using SwissDock (Grosdidier, et al., 2011, Nucleic Acids Res 39, W270-W277) (FIG. 11C). Although bioinformatics predicted successful binding, no expression of the anti-CD90 scFv could be achieved (data not shown). Comprehensive comparison of the sequences with the database revealed two cysteine residues (position 31 and 59) that were not found in any other reference and were not predicted to form disulfate bonds running SAbPred modeling (Dunbar, et al., 2016, Nucleic Acids Res 44, W474-W478). Cysteines were therefore exchanged (position 31 Tyrosine [Y], position 59 Arginine [R]) to match the reference sequences. In addition, the linker was exchanged to an alternate linker sequence (GSDSNAGHASAGNTS) (SEQ ID NO: 66) (Anliker, et al., 2010, Nature Methods 7, 929-935) to enable easier production and sequencing of variants.

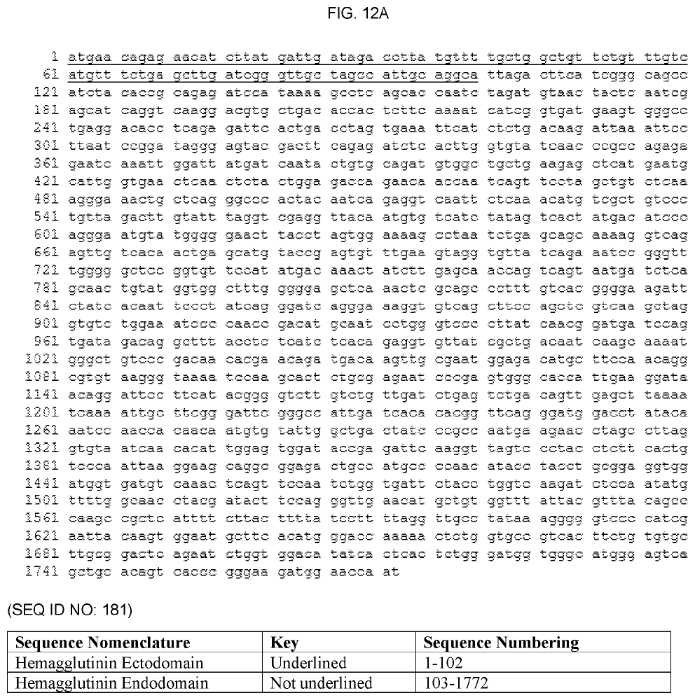

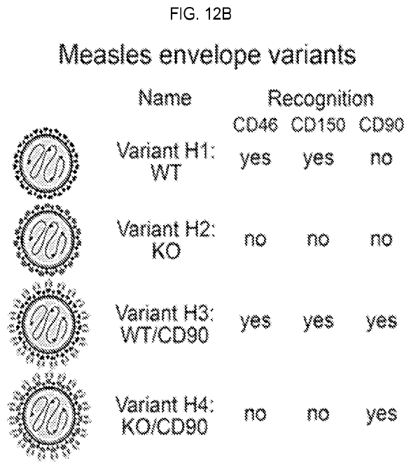

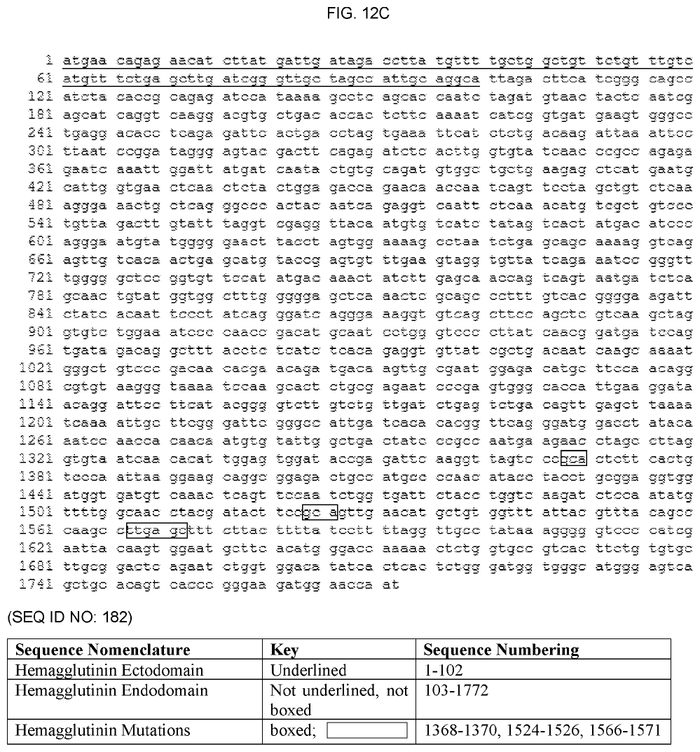

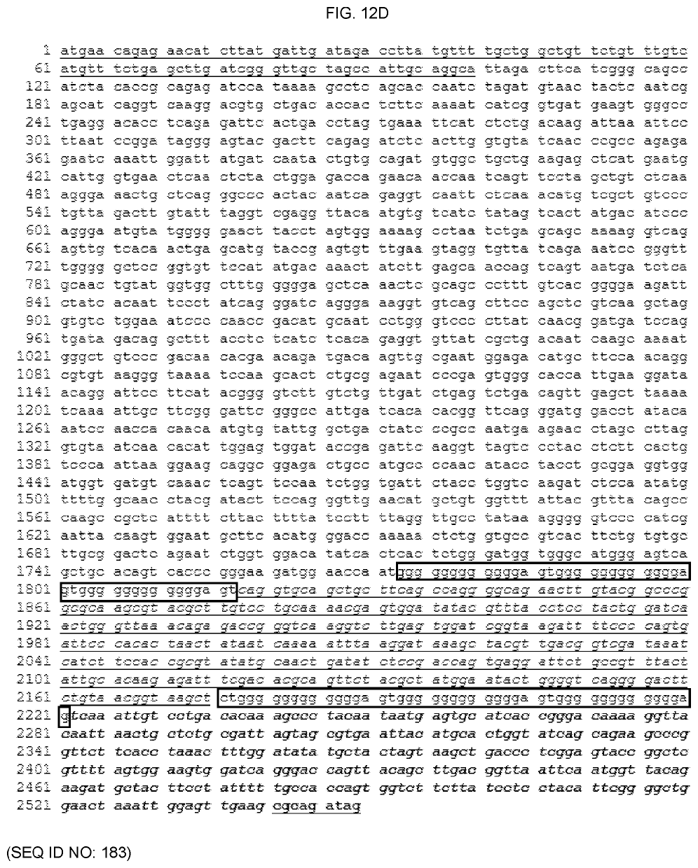

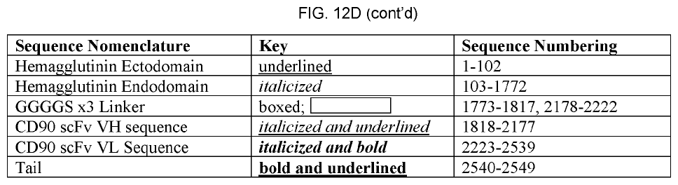

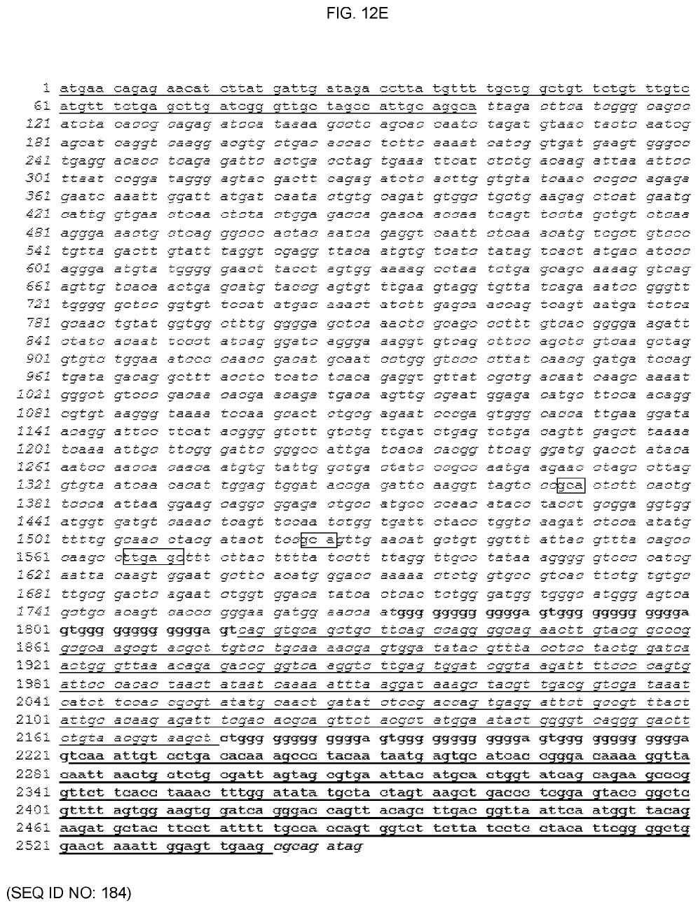

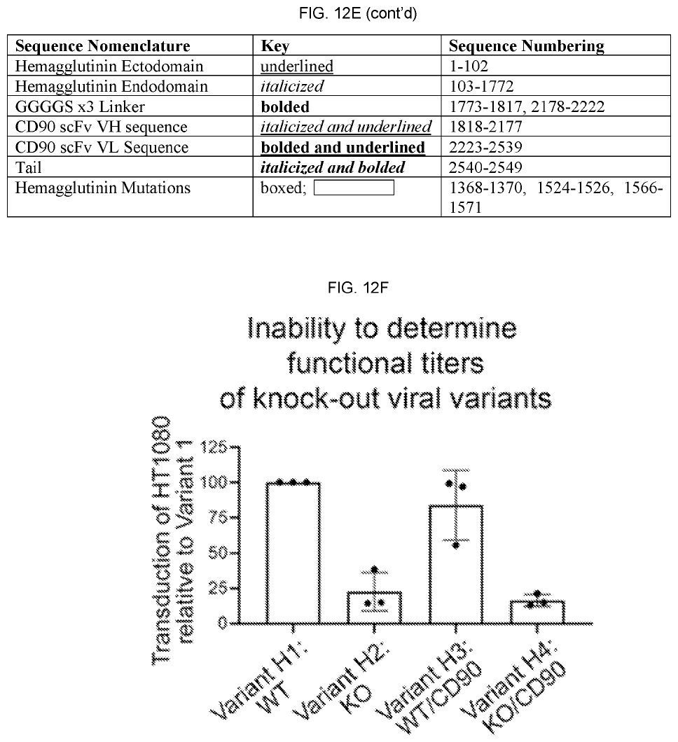

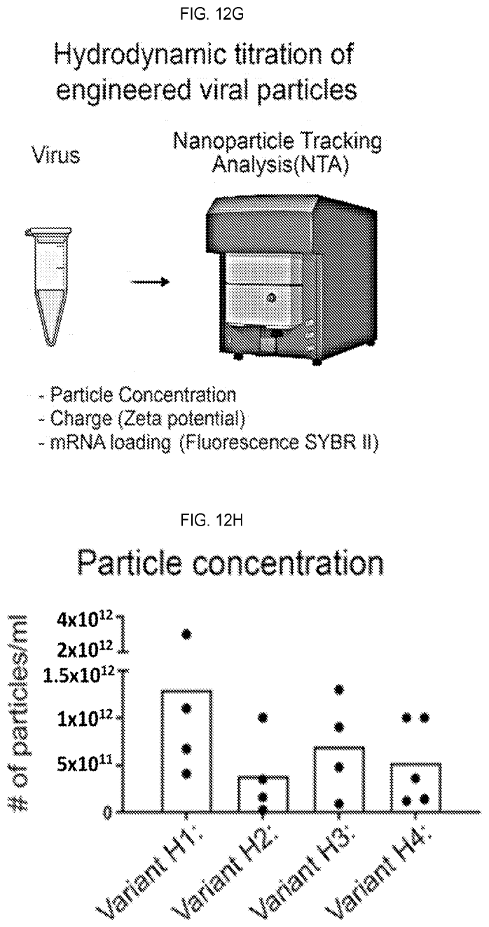

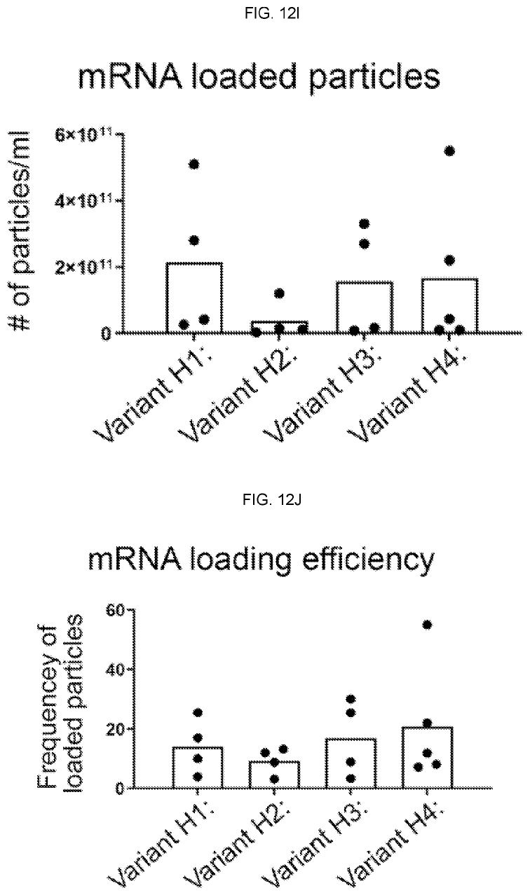

Design, Production, and Hydrodynamic Titration of CD90-Targeted Measles-Pseudotyped Viral Vectors. Measles-pseudotyped lentiviral vectors efficiently transduce a wide variety of cell types that recognize the broadly expressed cell surface antigens CD46 and CD150 (Levy, et al., 2017, Blood Adv 1, 2088-2104; and Frecha, et al., 2008, Blood 112, 4843-4852). Here the measles envelope was stepwise developed to remove the native targeting and instead exclusively recognize CD90 as previously described for other target cells (Anliker, et al., 2010, Nature Methods 7, 929-935). In addition to the wildtype envelope (Variant H1: WT, FIG. 12A), three variants of the measles envelope were generated (FIG. 12B). To remove its ability to target CD46/150 and thus create a virus that should not be able to elicit fusion and transduce cells (Funke, et al., 2008, Mol Ther 16, 1427-1436), four modifications were introduced to the amino acid sequence of the measles glycoprotein (Variant H2: knockout (KO), FIG. 12C). A variant was created that is capable to detect CD46, CD150, and CD90, inserting the CD90 scFv at the end of the glycoprotein (Variant H3: WT/CD90, FIG. 12D). Finally, the CD46/CD150 KO and CD90 recognition was combined to lead to a variant that should only bind and fuse on CD90-expressing cells (Variant H4: KO/CD90, FIG. 12E).

Due to the inability of the KO variants to be titrated on standard cell lines such as HT1080 (FIG. 12F), a methodology was developed using hydrodynamics combined with fluorescence to determine the number of total as well as RNA-filled viral particles. Size and number of viral particles were determined using the ZetaView@(Particle Metrix), a Nanoparticle Tracking Analysis (NTA) instrument for measuring particle size, zeta potential, concentration, and fluorescence (FIG. 12G). Regardless of the modifications and addition of the CD90 scFv, all four envelope variants equally produced viral particles (FIG. 12H). Successful loading of viral capsids and loading efficiency was determined staining the particles with SYBR 1l. The number of RNA-loaded viral particles was highly variable across batches (FIG. 12I). However, the loading efficiency of viral particles (frequency of RNA-loaded particles within total particles) was very consistent, averaging at 10% to 20% with no obvious differences due to the modification made (FIG. 12J).

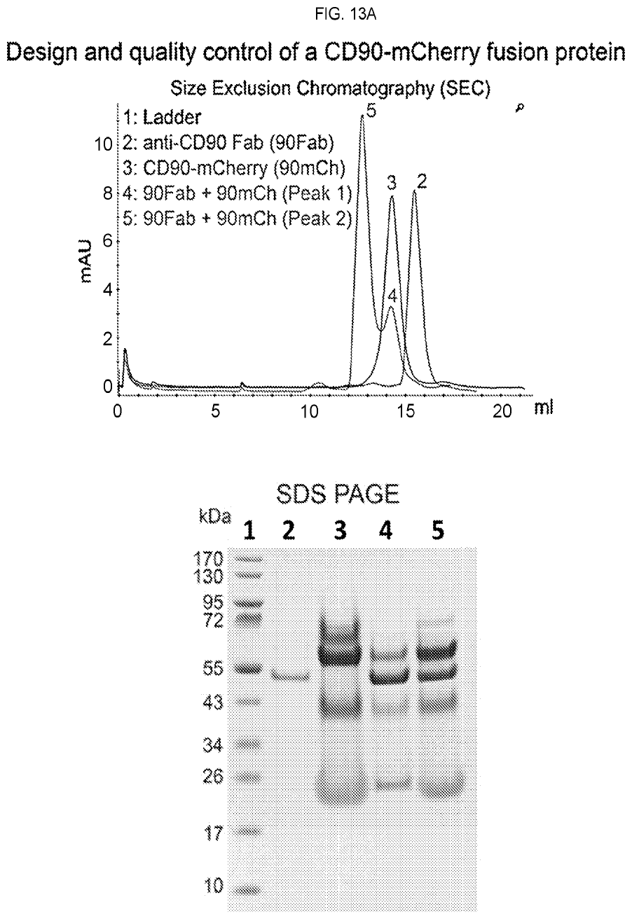

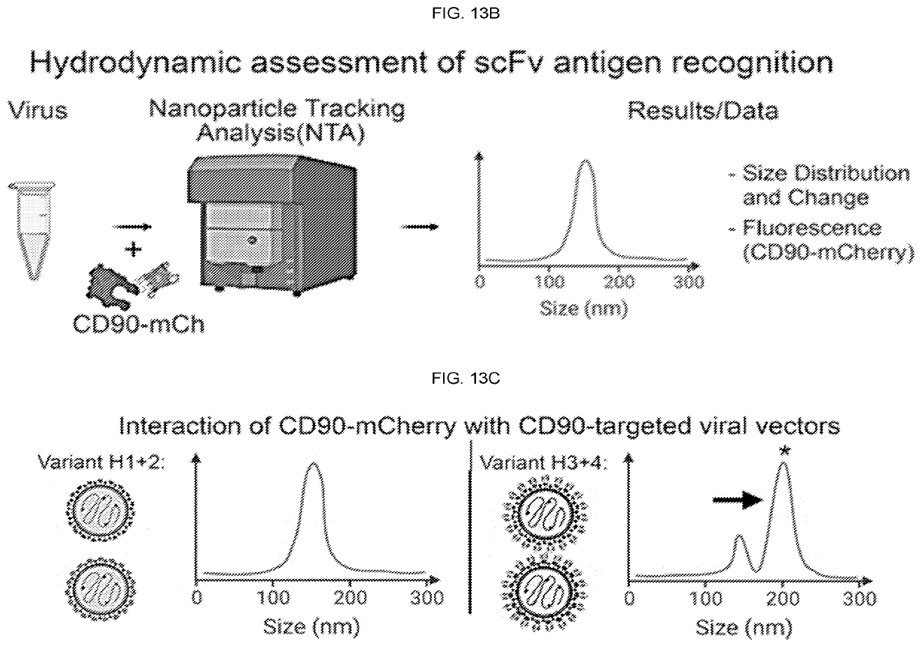

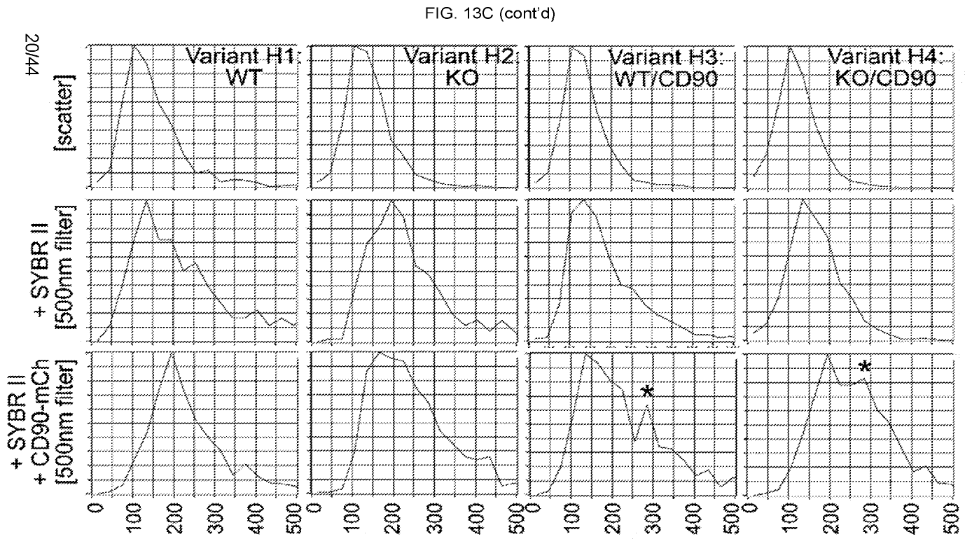

CD90 scFv on Viral Vectors Bind CD90 Protein. To ensure the ability of CD90-targeted viral vectors to recognize CD90 antigen, a custom assay was designed using NTA. The extracellular domain of CD90 was fused with the fluorochrome mCherry (CD90-mCh) and the fusionprotein produced with the Daedalus system (Bandaranayake, et al., 2011, Nucleic Acids Res 39, e143). The quality of the CD90-mCh fusion protein was confirmed via its interaction with anti-CD90 Fabs from the hybridoma cell line measured through size exclusion chromatography (SEC) using an A200 column run in PBS buffer. Each protein exhibited a single peak at the expected molecular weight (MW) (FIG. 13A). Fab binding to CD90-mCh was confirmed by the shift of the elution peak to a larger MW species and by SDS-PAGE of the peak showing presence of both proteins.

Next, the size of the vector variants were measured in the presence and absence of the CD90-mCh protein (FIG. 13B). All four vectors were very similar in size (range 110-130 nm) regardless of the modifications made measuring the scattered light (FIG. 13C, scatter, top row). Adding SYBR I to stain only RNA-loaded particles and observing the fluorescence in the 500 nm filter, particles appeared slightly larger across all four variants and a broader size range due to variability in RNA loading (FIG. 13C, +SYBR II, middle row). Finally, vectors incubated with CD90-mCh protein were measured (FIG. 13C, +SYBR II+CD90-mCh, bottom row). Due to the background noise of unbound protein in the 600 nm filter, measurements were performed in the 500 nm filter focusing only on RNA-loaded particles. CD90-decorated viral variants, H3 and H4 with CD90-mCh, formed new peaks at 300 nm, indicating formation of a complex. Only a minor change in size was seen for variant H1 and H2, which lacked the CD90 scFv, due to additional background noise signal from the CD90-mCh protein.

In summary, an anti-CD90 scFv was successfully engineered onto the measles envelope without impairing the vector production and maintaining viral titers. Most importantly, anti-CD90 scFvs on the viral capsid are fully functional and able to recognize CD90 antigen in the custom hydrodynamic readout confirming CD90 specificity of the scFv.

Targeted Viral Vectors Transduce CD90-Expressing Jurkat Cells and Human HSCs. The measles envelope has been extensively modified and previous attempts to use similar systems to specifically transduce CD34+ cells failed due to the lack of fusion with the target cells (Benedict, et al., 1999, Hum Gene Ther 10, 545-557). Consequently, the CD90-targeted viral vectors were tested for fusion and transduction ability. For the initial tests, CD90-expressing Jurkat cells were used and incubated with all four viral variants. Jurkat cells were kept in culture for 5-7 days to guarantee stable integration of the transgene and wash out transiently delivered fluorescent protein sticking on the viral particles.

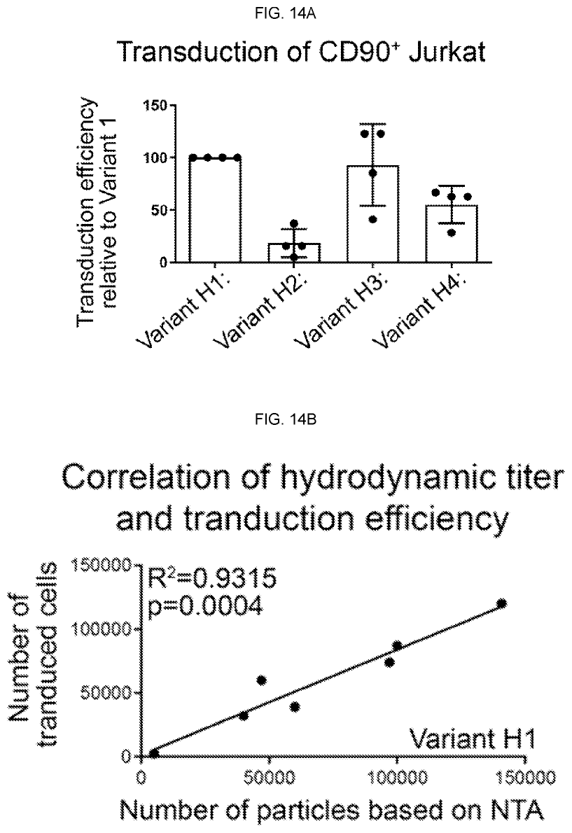

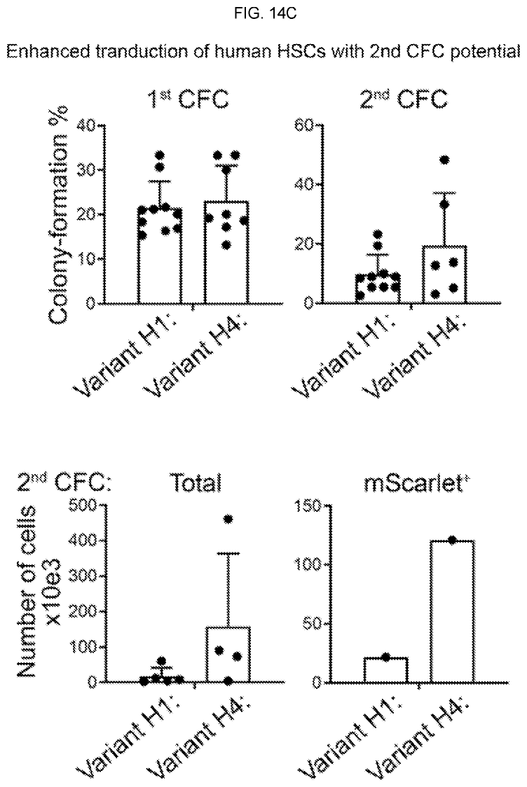

Determined by flow-cytometry, transduction of Jurkat cells were observed with variant H1 (FIG. 14A). Relative to variant H1, knock-out of CD46 and CD150 recognition reduced the ability of variant H2 to transduce Jurkat cells by 81.6±13.4% (FIG. 14A). Addition of the CD90 scFv on the H WT virus (variant H3) had no impact on the transduction ability in comparison to the WT variant H1. Finally, variant H4 (KO/CD90) regained transduction capability over variant H2 (KO), reaching on average 44.8±17.9% transduction efficiency. The reliability was further evaluated of hydrodynamic titers determined with NTA for variant H1 (FIG. 14B). A very high correlation (R²=0.9315) was found in between the number of mRNA-loaded particles and the number of transduced Jurkat cells.

Next, human GCSF-mobilized CD34⁺ cells were transduced with variant H1 and H4 vectors to analyze the targeting efficiency of the CD34⁺CD90⁺ subset (FIG. 14C). Due to the internalization/blocking and resulting inability to detect CD90 surface expression after exposure to the variant 4 virus by flow-cytometry, mScarlet expressing CD34⁺ cells from both conditions were FACS-purified and introduced into colony-forming cell (CFC) assays to functionally determine differences in the targeting of both viruses. No differences in the total colony-formation were seen comparing the WT and CD90-targeted virus in primary CFCs, whereas more colonies were found in secondary CFCs when the targeted virus was used, indicating selective transduction of more primitive human HSPCs with enhanced proliferation potential with variant 4. Colonies in secondary CFC assays from variant H4-transduced cells contained more total cells as well as a greater number of stably mScarlet-expressing cells, confirming greater proliferation and expansion potential of cells transduced with the CD90-targeted virus.

These data confirm that CD90-targeted viral vectors are capable of recognizing CD90 antigen on the surface of Jurkat cells as well as human HSCs and effectively fuse with the cells. Furthermore, hydrodynamic titers were highly predictable for the transduction efficiency, confirming accuracy and validity of our new titration methodology for surface-engineered viral vectors.

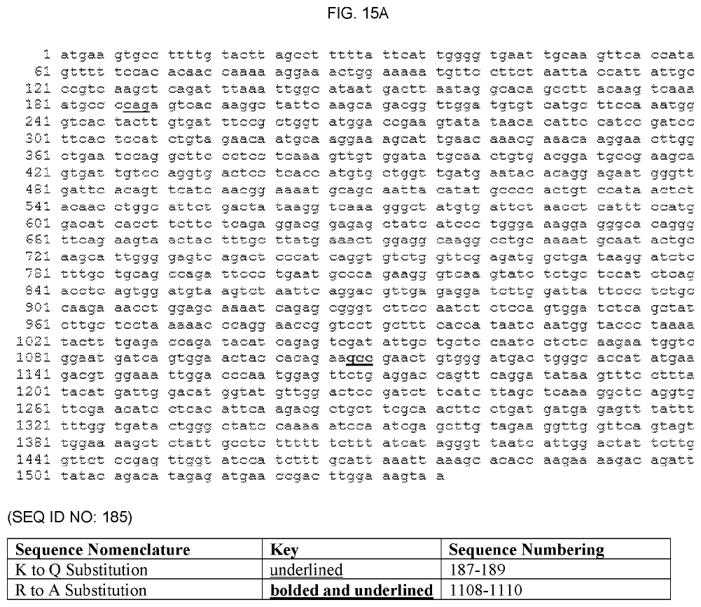

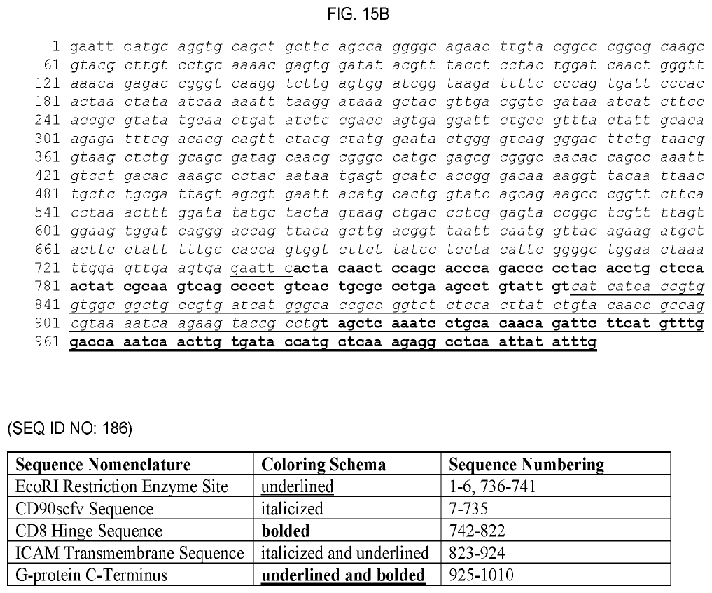

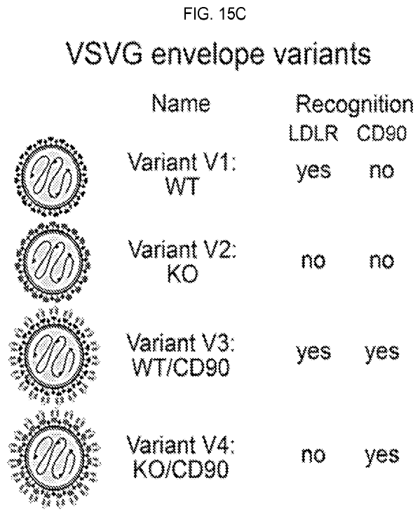

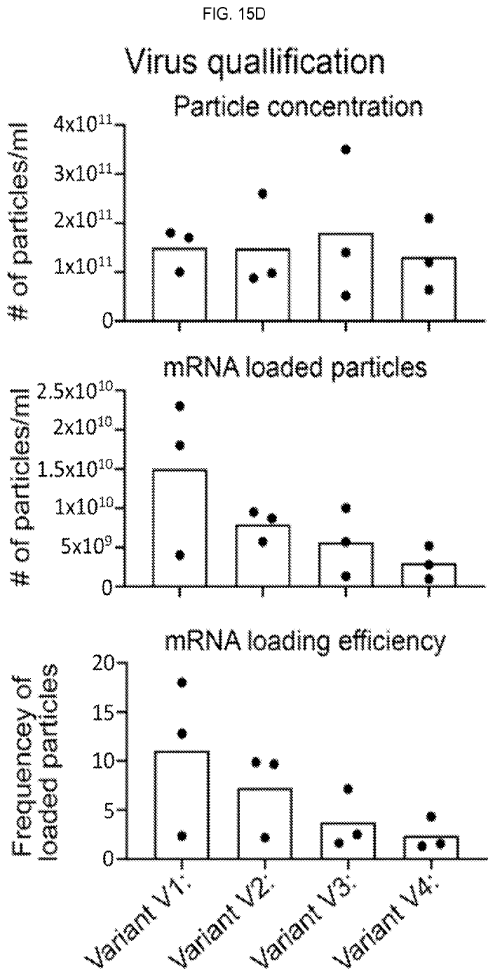

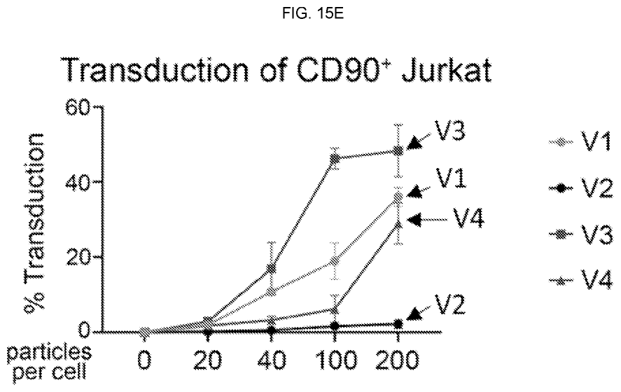

VSV-G-Based Viral Vectors Can Be Targeted to CD90. In addition to measles-pseudotyped viral vectors, the VSV-G-based system was evaluated for surface engineering following the previously described strategy by Dobson et al. Dobson, et al., 2022, Nat Methods 19, 449-460 Native targeting of the VSV-G envelope for the LDL-receptor (LDL-R) were further knocked out to introduce specific point mutations as described by Nikolic et al (FIG. 15A) (Nikolic, et al., 2018, Nat Commun 9, 1029). The CD90 scFv was introduced onto the capsid using a CD8 hinge and ICAM domain (FIG. 15B). Similar to the measles vectors, four VSV-G variants were generated with V1 (WT) recognizing LDL-R, V2 (KO) removing LDLR recognition, V3 (WT/CD90) recognizing LDL-R and CD90, and V4 (KO/CD90) only binding to CD90 (FIG. 15C).

The number of total viral as well as RNA-loaded particles was determined using the ZetaView® instrument (FIG. 15D). Regardless of the modifications and addition of the CD90 scFv, all four envelope variants equally produced viral particles. However, the loading efficiency of viral particles (frequency of RNA-loaded particles within total particles) decreased as more engineering was done with the lowest loading efficiency seen in V4.

To test functionality of the surface-engineered VSV-G vectors, K562 cells were exposed to increasing doses of all four viruses (FIG. 15E). Similar to the measles variants, the V2 (KO) variant failed to fuse with the cells, the V3 (WT/CD90) version demonstrated a gain of function relative to V1 (WT), and V4 (KO/CD90) regained the ability to fuse with cells comparable to V1 (WT). Transduction efficiency of K562 for variants V1, V3, and V4 was dose-dependent, with no unwanted uptake seen for V2 even in the highest dose.

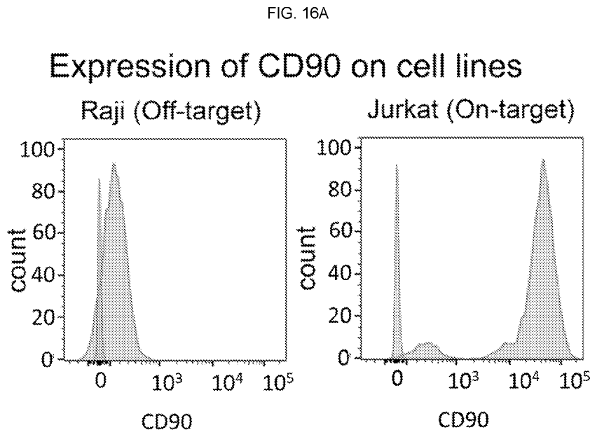

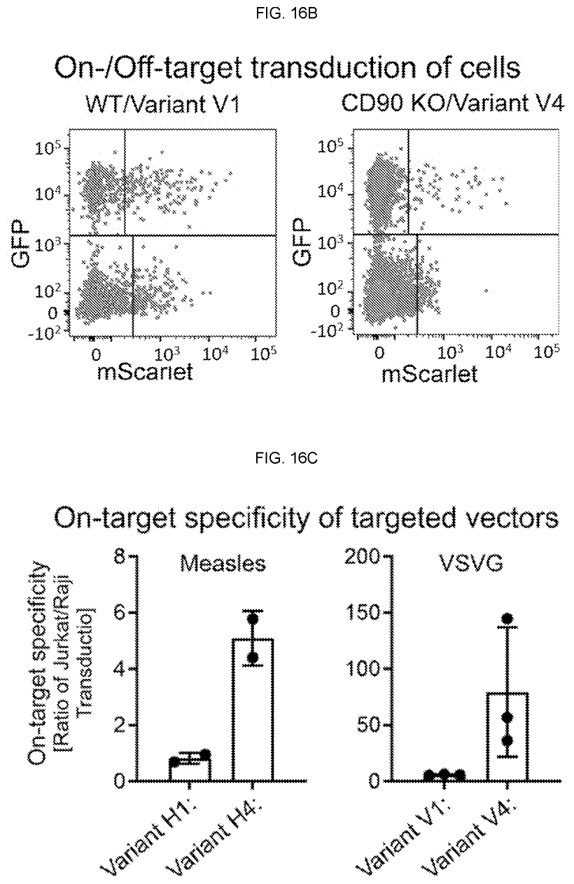

On-Target Efficiency of CD90-Targeted Viral Vectors. To test the on- and off-target activity of the CD90-targeted measles—as well as VSV-G-pseudotyped viral vectors, co-culture experiments were performed mixing CD90-lacking (off-target) and CD90-expressing (on-target) suspension cell lines. Raji cells, human B lymphoblastoid cells, do present very low levels of CD90 protein on the cell surface (off-target), whereas Jurkat cells, immortalized human T lymphocytes, express CD90 protein at high levels (on-target) (FIG. 16A). GFP-transduced Jurkat cells and GFP⁻ Raji cells were mixed and exposed to measles and VSV-G variants 1 and 4 in a serum-free, transduction enhancer-free, suspension culture system, and mScarlet expression flow-cytometrically determined on day 3-5 post-transduction (FIG. 16B). When the variant H1/V1 (WT) virus was used, mScarlet expression was seen for Raji as well as Jurkat cells by flow cytometry. However, when the cell mix was exposed to variant H4/V4 (KO/CD90), a strong preference of mScarlet signal was seen in the CD90⁺ Jurkat cells (on-target) and only minimal transduction in the CD90^{low/-} Raji cells (off-target) (FIG. 16B). To quantify the on-target specificity, the ratio was calculated of mScarlet⁺GFP⁻ cells within total GFP⁻ Raji to mScarlet⁺GFP⁺ cells within total GFP⁺ Jurkat cells (FIG. 16C). For both virus types, the CD90-targeted variant 4 showed significantly higher on-target specificity in comparison to the variant 1 (WT) confirmed CD90-mediated target specificity.

Discussion Here, the successful design of CD90-targeted measles- and VSV-G-based viral vectors equipping viral capsids was demonstrated with a novel anti-CD90 scFv. Surface engineering did neither impact titers, binding, nor fusion of viral vectors to CD90-expressing cell lines and primary human HSCs. Most importantly, targeted vectors demonstrated enhanced on-target specificity in mixed cultures of cell lines as well as for human HSCs within bulk CD34⁺ HSPCs ex vivo. Novel NTA-based methods were further described to reliably validate surface engineered viral vectors measuring their size, loading-efficiency, and targeting functionality. Hydrodynamic titration of engineered vectors is highly reproducible and predictive of the transduction efficiency, providing a reliable surrogate to functional readouts on cell lines which are not applicable to surface engineered vectors.

The development of targeted gene therapy agents has been very successful for non-hematological diseases (Kedmi, et al., 2018, Nat Nanotechnol 13, 214-219; De, et al., 2018, Hum Gene Ther Methods 29, 146-155; van Haasteren, et al., 2018, Expert Opin Biol Ther 18, 959-972; Kleinlutzum, et al., 2017, Front Oncol 7, 127; and Wei, et al., 2020, Nat Commun 11, 3232). Previous work has shown that by conjugating antibodies or scFvs to the surface of nanoparticles (NPs) and viruses, one can reliably target cells of interest (Benedict, et al., 1999, Hum Gene Ther 10, 545-557; Brendel, et al., 2015, Mol Ther 23, 63-70; Frecha, et al., 2008, Blood 112, 4843-4852; Kedmi, et al., 2018, Nat Nanotechnol 13, 214-219; Kleinlutzum, et al., 2017, Front Oncol 7, 127; Niu, et al., 2018, Biomaterials 167, 132-142; Rosenblum, et al., 2020, Sci Adv 6; Zheng, et al., 2013, Journal of Controlled Release 172, 426-435; Kneissl, et al., 2012, PLoS One 7, e46667; Moffett, et al., 2017, Nat Commun 8, 389; and Veiga, et al., 2018, Nat Commun 9, 4493). Targeted delivery however depends on the uptake of the vector after attaching to the cell/antigen, which could not be achieved with CD34-targeted HSC gene therapy vectors (Benedict, et al., 1999, Hum Gene Ther 10, 545-557). Here, CD90-targeted viral vectors were designed that successfully fused and transduced CD90-expressing cell lines as well as CD34⁺CD90⁺ HSCs with secondary colony-forming potential. Although the exact mechanism of CD90-mediated cellular uptake is not known, cytomegalovirus (CMV) and Human Immunodeficiency Virus (HIV) use CD90 as a crucial part of their cellular entry process (Rudnicka, et al., 2009, J Virol 83, 6234-6246; Li, et al., 2016, J Virol 90, 9766-9781; and Li, et al., 2015, PLoS Pathog 11, el004999). CMV entry was interrupted when CD90 was either blocked with an antibody or downregulated using an siRNA approach (Li, et al., 2015, PLoS Pathog 11, e1004999). In addition, liposomes conjugated with anti-CD90 antibodies have been shown to be capable of binding and internalizing through the CD90 antigen on liver cancer cells, demonstrating efficient uptake of CD90-targeted lipid nanoparticles (LNPs) also in vivo (Yang, et al., 2016, Oncotarget 7, 35894-35916). Disclosed viral vectors have been genetically modified to lose their native binding (LDL-R for VSV-G; CD46 and CD150 for measles), but their ability to fuse with the target cell remains intact. The CD90 scFv is therefore likely acting as a binder, whereas cellular entry remains to be dependent on viral capsid. More in-depth studies can determine whether CD90 is actively involved in the uptake, which pathways are triggered, and whether this system can also be used completely independent from viral fusion proteins. Regardless of the exact pathways involved, CD90 protein was internalized on primary human HSCs without any obvious impact on the cells proliferation and differentiation potential.

A major bottleneck for the development of surface engineered viral vectors for HSC gene therapy is the inability to quickly and reliably verify the vectors before use. Genetically engineered viral vectors lose their native binding and functional titration methods on HEK293T or HT1080 cell lines are not possible. In addition, validation of antigen/target recognition is needed to ensure that surface-engineered viral vectors are functional and targeting ligands have been successfully integrated into the capsid. To close this gap, a novel methodology was developed to titrate and validate viral vectors using nanoparticle tracking analysis (NTA). NTA allows for the efficient and reliable quantification of viral vector concentration, size, and mRNA loading. Most importantly, hydrodynamic viral titers correlated with the transduction efficiency on cell lines for wildtype variants, providing a surrogate method for the traditional titration on cell lines or other qPCR-based strategies. NTA was further used to demonstrate successful antigen recognition of targeted vectors. Custom-designed fluorochrome-conjugated CD90 protein was bound by the scFv-decorated VSV-G- and measles-pseudotyped viral vectors, resulting in a size increase measured by NTA and confirming fully functional antigen recognition when the targeting ligand was presented on the virus. The measurement of these parameters is nearly instantaneous and provides a new alternative to traditional titration methods of wildtype, novel surface-engineered viral vectors, as well as virus-like particles. As viral vectors often undergo freezing and thawing, quality control and quantification of viral particles can even be performed right before applying them to the cells or injecting them in vivo, providing an opportunity to adjust and carefully control viral titers in real-time.

A major advantage of the disclosed CD90-targeted viral vectors is the ability to incorporate them into already existing ex vivo gene therapy pipelines focusing on the gene-modification of CD34⁺ HSPCs. Further purification of the CD90⁺ subset for direct targeting and to enhance the transduction efficiency (Radtke, et al., 2020, Mol Ther Methods Clin Dev 18, 679-691) would potentially no longer be needed to transduce long-term engrafting HSCs and likely require even lower viral titers reducing the overall costs. Even the purification of CD34⁺ cells from the leukapheresis products may no longer be necessary, providing a novel vector to realize closed-system vein-to-vein applications with the transduction of cells in bags and immediate infusion of the transduced cells back into the patient. Finally, direct intravenous or intraosseous injection of the HSC-targeted viral vectors would be warranted to further simplify HSC gene therapy, providing full portability, and consequently better accessibility.

The disclosed CD90-targeted vectors improve already existing ex vivo HSC gene therapies as well as overcome current hurdles to apply gene therapy in vivo. The development of targeted viral vectors increases the on-target efficiency, safety, and accessibility of HSC gene transfer for a variety of different diseases and will be a crucial step in democratizing access to gene therapies.

Materials and Methods. mRNA extraction, cDNA synthesis, and heavy/light chain sequencing of the hybridoma cells. mRNA was extracted from the hybridoma cells using an RNAeasy kit (Qiagen USA, MD). Extracted mRNA was run on TapeStation (Agilent Technologies, Santa Clara, CA) for integrity. cDNA was generated from mRNA using an applied biosystem high capacity RNA-to-cDNA kit (Thermo Fisher Scientific, Waltham, MA). Primers for the variant regions of the antibody were acquired from the literature (Sun, et al., 2012, World J Microbiol Biotechnol 28, 381-386) and ordered using IDT. CD90 ab internal reverse primer (TTC AGT CAC CAT GCT GTT GAC) (SEQ ID NO: 187) forward primer (CCA GCA GAA GCC AGG ATC) (SEQ ID NO: 188). Antibody variable regions were sequenced using on an Applied Biosystems 3730x1 DNA Analyzers (Thermo Fisher Scientific, Waltham, MA). Raw sequencing data was analyzed using International ImMunoGeneTics V-QUEry and STandardization program to align and annotate the antibody sequence.

Modeling and visualization of proteins. The antibody structure was predicted with SabPred (Dunbar, et al., 2016, Nucleic Acids Res 44, W474-W478). Antibody-protein docking was predicted using Swissdock (Grosdidier, et al., 2011, Nucleic Acids Res 39, W270-W277). Visualizations were generated with Pymol (The PyMOL Molecular Graphics System, Version 2.0 Schrödinger, LLC).

Cloning of virus plasmids. Measles plasmid were provided by Els Verhoeyen (Levy, et al., 2017, Blood Adv 1, 2088-2104). Cloning was performed using restriction enzymes from New England Biolabs ([NEB] Ipswich, MA) and Gibson Assembly Master Mix (NEB). DNA tiles containing the desired sequences to be cloned into the plasmids were acquired from Codex DNA (Codex DNA, San Diego, CA). Cloned products were confirmed via sequencing on an Applied Biosystems 3730xl DNA Analyzers (Thermo Fisher Scientific, Waltham, MA). The relevant sequences are in FIGS. 12A, 12C-12E, 15A, and 15B.

VSV-G Knockout (KO) and targeted binder plasmids were provided by the Baker Laboratory (Institute of Protein Design, University of Washington, Seattle, WA). Cloning of CD90 scFv into the targeted binder plasmid was performed by Eco721-FastDigest (Thermo Fisher Scientific, cat #FD0364) and Eco1471-FastDigest (Thermo Fisher Scientific, cat #FD0424) restriction enzyme digest of targeted binder plasmid followed by Gibson Assembly (NEB, cat #E2611S) with synthesized Gibson block sequence (Integrated Data Technologies, Coralville, IA) containing the CD90 scfv sequence (FIGS. 12D, 12E, and 16A-16C). Successful cloning was confirmed by EcoRI (NEB, cat #R3101 S) restriction enzyme digest, followed by sequencing on Applied Biosystems 373xl DNA Analyzers (Thermo Fisher Scientific, Waltham, MA).

Virus production. HEK293T cells were expanded on gelatin coated plates and transfected with plasmids mixed with polyethylenimine (PEI) (Polysciences, Warrington, PA) at a ¼ μg plasmid to ug PEI ratio. Measles virus plasmid ratios were 27 μg pLenti-EF1a-mScarlet-I-WPRE transfer plasmid, 17 μg 2^{nd} generation Gag/Pol Helper plasmid pCMVdelta8.74, 10 μg measles hemagglutinin Delta 24H, and 10 μg measles fusion Delta F per 15 cm cell plate. VSV-G virus plasmid ratios were 27 μg pLenti-EF1a-mScarlet-I-WPRE transfer, 17 μg 2^{nd} generation Gag/Pol Helper plasmid pCMVdelta8.74, 10 μg VSV-G fusion, and 10 μg of relevant targeting ligand plasmid per 15 cm cell plate. Twenty-four (24) hours after transfection, the media was changed to LV-MAX (Thermo Fisher Scientific) media with 20 mM HEPES (Thermo Fisher Scientific) and 1× L-glutamine (Thermo Fisher Scientific). Virus production media was allowed to sit for a further 24 hours, then the supernatant was collected, spun down at 800 g to remove cellular debris, filtered with an 8 μm filter, and spun down for 24 hours at 4800 g. The concentrated supernatant was resuspended at a 1/100 volume.

Hydrodynamic titration. Hydrodynamic titration of viral particles was performed on a Zetaviewer machine (Particle Metrix, GmbH, Inning am Ammersee, Germany). The machine was calibrated with a 100 nm bead test sample prior to all measurements. Viruses were diluted 1 to 1000 in molecular grade water and analyzed for their size characteristics. Readouts from the machine were back calculated to account for dilution. Diluted virus samples were next incubated with 1×SYBR II Green RNA stain (Thermo Fisher Scientific) for 5 minutes before running on the Zetaviewer with a 488 nm filter in place to only identify virus particles loaded with mRNA and stained with SYBR II. Additionally, virus's targeted to CD90 were incubated with SYBR II and CD90-mCherry protein for 30 minutes. Virus-protein complexes were analyzed on the Zetaviewer with a 488 or 520 nm filter in place to capture particles loaded with mRNA carrying CD90-mCherry.

CD90 fab production. Anti-CD90 IgGs were produced by the Antibody Technology Core at the Fred Hutchinson Cancer Center from the previously reported hybridoma cell lines producing the CD90 antibody clone 5E10 (Craig, et al., 1993, J Exp Med 177, 1331-1342). Fab fragments were generated from full-length CD90 antibodies with immobilized papain (Thermo Fisher Scientific Cat. Number 20341) per manufacture protocol. After verifying full digestion by SDSPAGE, the reaction mixture was buffer exchanged into PBS and the Fab separated from the Fc by anion exchange chromatography using a HiTrap Q column on an AKTA Pure.

CD90-mCherry production. The CD90-mCherry Fusion protein was expressed in HEK293 cells as the following amino acid sequence:

(SEQ ID NO: 189)
METDTLLLWVLLLWVPGSTGQKVTSLTACLVDQSLRLDCRHENTSSSPI
QYEFSLTRETKKHVLFGTVGVPEHTYRSRTNFTSKYNMKVLYLSAFTSK
DEGTYTCALHHSGHSPPISSQNVTVLRDKLVKCEGGSGGGSGGGVSKGE
EDNMAIIKEFMRFKVHMEGSVNGHEFEIEGEGEGRPYEGTQTAKLKVTK
GGPLPFAWDILSPQFMYGSKAYVKHPADIPDYLKLSFPEGFKWERVMNF
EDGGVVTVTQDSSLQDGEFIYKVKLRGTNFPSDGPVMQKKTMGWEASSE
RMYPEDGALKGEIKQRLKLKDGGHYDAEVKTTYKAKKPVQLPGAYNVNI
KLDITSHNEDYTIVEQYERAEGRHSTGGMDELYK*

The DNA was codon optimized for human expression, cloned into a modified lentivirus expression system, and expressed as described in Bandaranayake et al. (Bandaranayake, et al., 2011, Nucleic Acids Res 39, e143) The media containing the secreted CD90-mCherry was concentrated, and purified using a P200 SEC column equilibrated in PBS buffer.

Cell lines. Jurkat (ATCC CLR-2899) cells, Raji (ATCC CCL-86), and K-562 (ATCC CCL-243) cells were all cultured in Roswell Park Memorial Institute (RPMI) with Penicillin Streptomycin at a final concentration of 50 to 100 I.U./mL (Thermo Fisher Scientific) and supplemented with 10% cosmic calf serum (Thermo Fisher Scientific).

Primary human HSCs. Primary human CD34⁺ cells were purchased from the Co-operative Center for Excellence in Hematology (CCEH) at the Fred Hutchinson Cancer Center (Fred Hutch). Collections were performed according to the Declaration of Helsinki and were approved by a local ethics committee/institutional review board of Fred Hutch. All healthy adult donors were mobilized with GCSF. Human CD34⁺ cells were enriched as previously described on a CliniMACS Prodigy according to the manufacturer's instructions (Miltenyi Biotec, Bergisch Gladbach, Germany).

Human CD34⁺ cells were cultured in StemSpan (STEMCELL Technologies, Vancouver, BC, Canada) medium supplemented with penicillin-streptomycin (PS) (100 U/mL) (Gibco by Life Technologies, Waltham, MA, USA) and 100 ng/mL of each stem cell factor (PeproTech, Cranbury, NJ, USA), thrombopoietin (TPO; PeproTech), and Fms-related tyrosine kinase 3 ligand (FLT3-L; Miltenyi Biotec). Cells were cultured at 37° C., 85% relative humidity, and 5% C02.

Virus transduction. Cells (Jurkat, Raji, human HSCs) were resuspended in serum-free media at a density of 150,000 cells per 150 μL per well of a 96-well plate (Corning, Corning, NY, USA). Concentrated viral supernatant was added to the cells and incubated for 8-12 hours. After transduction cells were washed and media replaced with relevant growth media specific to cell type (see above). Transgene expression was determined flow-cytometrically 72-120 hours post-transduction.

Viability assessment and counting of human CD34+ cells and cell lines. The cell viability was analyzed using the Countess II FL automated cell counter (Thermo Fisher Scientific). A 10 μL volume of trypan blue stain (0.4%) (Invitrogen, Waltham, MA, USA) was mixed with 10 μL of cell suspension, and 10 μL of the mixture was applied to a disposable cell counting chamber slide and inserted into the device. The percentage of cell viability of each sample was recorded in duplicate and reported as the mean±standard error of mean (SEM).

Flow cytometry analysis and cell sorting. Flow cytometric analysis and sorting of cell lines and human CD34+ cells were performed using the fluorochrome-conjugated antibodies listed in Table 4. Dead cells and debris were excluded via forward light scatter (FSC)/side light scatter (SSC) gating and DAPI staining. Flow cytometric analysis and cell sorting were performed on a FACSymphony A5, FACSCelesta, FACSAria IIu, and Symphony S6 (BD Biosciences, San Jose, CA). Data were acquired using FACSDiva version 6.1.3 and newer (BD Biosciences). Data analysis was performed using FlowJo version 8 and higher (BD Biosciences).

TABLE 4

| | | Catalog | Clone | | |
|---|---|---|---|---|---|
| Antigen | Vendor | Number | Name | Fluorochrome | Application |
| CD45 | BD | 560367 | HI30 | V450 | Human HSCs |
| | Biosciences | | | | |
| CD34 | BD | 561209 | 563 | APC | Human HSCs |
| | Biosciences | | | | |
| CD90 | BD | 328124 | 5E10 | PE-Cy7 | Human HSCs, |
| | biosciences | | | | Jurkat cells |
| | | | | | Raji Cells |

CFC assay. For CFC assays, 200 FACS-purified CD34⁺mScarlet⁺ cells were seeded on 30 mm plates in 1 mL of methylcellulose (MethoCult H4435, STEMCELL Technologies). Colonies were counted and scored after 12-14 days according to morphology into colony-forming unit (CFU)-granulocyte (CFU-G), CFU-macrophage (CFU-M), granulocyte-macrophage (CFU-GM), and burst-forming unit-erythrocyte (BFU-E). Colonies including erythroid and myeloid cells were scored as CFU-Mix. For secondary CFC assays, primary colonies were harvested, washed twice with PBS, and 5% of cell suspension replated into 1 mL of methylcellulose. Secondary colonies were counted and scored after 12-14 days.

### (x) Closing Paragraphs

The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (nonpolar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs.

Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.](1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of an antibody binding domain to its cognate binding molecule with an affinity or Ka (i.e., an equilibrium association constant of a particular binding interaction with units of 1/M) equal to or greater than 10⁵ M⁻¹, while not significantly associating with any other molecules or components in a relevant environment sample. “Specifically binds” is also referred to as “binds” herein. Binding domains may be classified as “high affinity” or “low affinity”. In particular embodiments, “high affinity” binding domains refer to those binding domains with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Ka of up to 10⁷ M⁻¹, up to 10⁶ M⁻¹, up to 10⁵ M⁻¹. Alternatively, affinity may be defined as an equilibrium dissociation constant (Kd) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Ka (equilibrium association constant) for the cognate binding molecule that is higher than the reference binding domain or due to a Kd (dissociation constant) for the cognate binding molecule that is less than that of the reference binding domain, or due to an off-rate (Koff) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

Each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically-significant reduction in binding between an antibody and antigen.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or +1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A modified antibody or antigen binding fragment thereof comprising
a variable heavy chain complementarity determining region (CDRH) 1 as set forth in SEQ ID NO: 41, a CDRH2 as set forth in SEQ ID NO: 42, and a CDRH3 as set forth in SEQ ID NO: 43, and
a variable light chain complementarity determining region (CDRL) 1 as set forth in SEQ ID NO: 171, a CDRL2 having the sequence ATS, and a CDRL3 as set forth in SEQ ID NO: 54 according to IMGT;
a CDRH1 as set forth in SEQ ID NO: 44, a CDRH2 as set forth in SEQ ID NO: 45, and a CDRH3 as set forth in SEQ ID NO: 46, and
a CDRL1 as set forth in SEQ ID NO: 173, a CDRL2 as set forth in SEQ ID NO: 56, and a CDRL3 as set forth in SEQ ID NO: 54 according to Kabat;
a CDRH1 as set forth in SEQ ID NO: 47, a CDRH2 as set forth in SEQ ID NO: 48, and a CDRH3 as set forth in SEQ ID NO: 46, and
a CDRL1 as set forth in SEQ ID NO: 173, a CDRL2 as set forth in SEQ ID NO: 56, and a CDRL3 as set forth in SEQ ID NO: 54 according to Chothia; or
a CDRH1 as set forth in SEQ ID NO: 49, a CDRH2 as set forth in SEQ ID NO: 50, and a CDRH3 as set forth in SEQ ID NO: 43, and
a CDRL1 as set forth in SEQ ID NO: 174, a CDRL2 as set forth in SEQ ID NO: 58, and a CDRL3 as set forth in SEQ ID NO: 54 according to North.

2. The modified antibody or antigen binding fragment of claim 1, wherein the variable heavy chain has the sequence set forth in SEQ ID NO: 40.

3. The modified antibody or antigen binding fragment of claim 1, wherein the variable light chain has the sequence set forth in SEQ ID NO: 145.

4. An antigen binding fragment of claim 1 comprising a single chain variable fragment (scFv).

5. The scFv of claim 4, wherein the heavy chain has the sequence as set forth in SEQ ID NO: 40.

6. The scFv of claim 4, wherein the light chain comprises the sequence as set forth in SEQ ID NO: 145.

7. The scFv of claim 4, wherein the linker sequence has the sequence as set forth in SEQ ID NO: 64, SEQ ID NO: 66, SEQ ID NO: 79, SEQ ID NO: 80, SEQ ID NO: 81, SEQ ID NO: 82. SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, or SEQ ID NO: 91.

8. The scFv of claim 4, wherein the linker sequence has the sequence as set forth in SEQ ID NO: 66.

9. The scFv of claim 4, wherein the linker sequence has the sequence as set forth in SEQ ID NO: 64.

10. The scFv of claim 4, having the sequence as set forth in SEQ ID NO: 156.

11. The scFv of claim 4, having the sequence as set forth in SEQ ID NO: 157.

12. The scFv of claim 4, linked to a viral structural protein.

13. The scFv of claim 12, wherein the viral structural protein comprises measles virus hemagglutinin (H), measles virus fusion (F), vesicular stomatis virus G (VSV-G), influenza hemagglutinin, ebolavirus glycoprotein (GP)1 or GP2, coronavirus spike, lassa virus glycoprotein precursor (GP) or spike, nipah virus NiV-G or NiV-F, rabies virus glycoprotein (G), respiratory syncytial virus fusion protein (F) or attachment protein (G), human immunodeficiency virus (HIV) gp41, HIV gp120, paramyxovirus F protein, filovirus GP2 protein, flavivirus E protein, alphavirus E1 protein, herpes simplex virus gH or gB protein, or arenavirus G2 protein.

14. The scFv of claim 12, wherein the viral structural protein comprises measles virus hemagglutinin.

15. The scFv of claim 14, wherein the measles virus hemagglutinin comprises a cytoplasmic tail truncation of up to 30 residues.

16. The scFv of claim 14, wherein the measles virus hemagglutinin comprises mutations Y481A, R533A, S548L, and F549S.

17. The scFv of claim 12, encoded by the sequence as set forth in SEQ ID NO: 158.

18. The scFv of claim 12, encoded by the sequence as set forth in SEQ ID NO: 159.

19. The scFv of claim 12, encoded by the sequence as set forth in SEQ ID NO: 160.

20. The modified antibody or antigen binding fragment of claim 1, comprising mutations in the Fc region.

21. The modified antibody or antigen binding fragment of claim 1, wherein the modified antibody or binding fragment thereof is humanized.

22. The modified antibody or antigen binding fragment of claim 1, wherein the modified antibody or binding fragment thereof is PEGylated.

23. The modified antibody or antigen binding fragment of claim 1, wherein the modified antibody or binding fragment thereof is conjugated to polyglutamic acid (PGA).

24. The modified antibody or antigen binding fragment of claim 23, wherein the PGA shields the positive charge of a polymeric nanoparticle.

25. The modified antibody or antigen binding fragment of claim 1, comprising M428L/N434S mutations.

26. The modified antibody or antigen binding fragment of claim 1, comprising G236A/S239D/A330L/1332E mutations.

27. The modified antibody or antigen binding fragment of claim 1, wherein the modified antibody or antigen binding fragment thereof is linked to a viral protein thereby providing a recombinant protein.

28. The modified antibody or antigen binding fragment of claim 27, wherein the viral protein comprises a structural protein.

29. The modified antibody or antigen binding fragment of claim 28, wherein the structural protein comprises measles virus hemagglutinin (H), measles virus fusion (F), vesicular stomatis virus G (VSV-G), influenza hemagglutinin, ebolavirus glycoprotein (GP)1 or GP2, coronavirus spike, lassa virus glycoprotein precursor (GP) or spike, nipah virus NiV-G or NiV-F, rabies virus glycoprotein (G), respiratory syncytial virus fusion protein (F) or attachment protein (G), human immunodeficiency virus (HIV) gp41, HIV gp120, paramyxovirus F protein, filovirus GP2 protein, flavivirus E protein, alphavirus E1 protein, herpes simplex virus gH or gB protein, or arenavirus G2 protein.

30. The modified antibody or antigen binding fragment of claim 1, wherein the modified antibody or antigen binding fragment thereof is linked to an toxin, a drug, a detectable label, a radioisotope, a nanoparticle, a bead, or a secondary binding domain.

31. The modified antibody or antigen binding fragment of claim 30, wherein the toxin comprises a holotoxin or a hemitoxin.

32. The modified antibody or antigen binding fragment of claim 30, wherein the drug comprises actinomycin D, anthracycline, auristatin, calicheamicin, camptothecin, CC1065, colchicin, cytochalasin B, daunorubicin, 1-dehydrotestosterone, dihydroxy anthracinedione, dolastatin, doxorubicin, duocarmycin, elinafide, emetine, ethidium bromide, etoposide, gramicidin D, glucocorticoids, lidocaine, maytansinoid (comprising monomethyl auristatin E [MMAE]; vedotin), mithramycin, mitomycin, mitoxantrone, nemorubicin, PNU-159682, procaine, propranolol, puromycin, pyrrolobenzodiazepine (PBD), taxane, taxol, tenoposide, tetracaine, trichothecene, vinblastine, vinca alkaloid, or vincristine.

33. The modified antibody or antigen binding fragment of claim 30, wherein the detectable label comprises a fluorescent protein, an enzyme label, fluorescent label, or a chemiluminescent label.

34. The modified antibody or antigen binding fragment of claim 33, wherein the fluorescent protein comprises blue fluorescent protein, cyan fluorescent protein, green fluorescent proteins, luciferase, orange fluorescent protein, red fluorescent protein, far red fluorescent protein, or yellow fluorescent protein.

35. The modified antibody or antigen binding fragment of claim 33, wherein the enzyme label comprises horseradish peroxidase, hydrolase, or alkaline phosphatase.

36. The modified antibody or antigen binding fragment of claim 33, wherein the fluorescent label comprises rhodamine, phycoerythrin, or fluorescein.

37. The modified antibody or antigen binding fragment of claim 30, wherein the radioisotope comprises actinium-225, iodine-131, arsenic-211, iodine-131, indium-111, yttrium-90, lutetium-177, astatine-211, bismuth-212, or bismuth-213.

38. The modified antibody or antigen binding fragment of claim 30, wherein the radioisotope comprises ²²⁸Ac, ¹¹¹Ag, ¹²⁴Am, ⁷⁴As, ²¹¹As, ²⁰⁹At, ¹⁹⁴Au, ¹²⁸Ba, ⁷Be, ²⁰⁶Bi, ²⁴⁵Bk, ²⁴⁶Bk, ⁷⁶Br, ¹¹C, ⁴⁷Ca, ²⁵⁴Cf, ²⁴²Cm, ⁵¹Cr, ⁶⁷Cu, ¹⁵³Dy, ¹⁵⁷Dy, ¹⁵⁹Dy, ¹⁶⁵Dy, ¹⁶⁶Dy ¹⁷¹Er, ²⁵⁰Es, ²⁵⁴Es, ¹⁴⁷Eu, ¹⁵⁷Eu, ⁵²Fe, ⁵⁹Fe, ²⁵¹Fm, ²⁵²Fm, ²⁵³Fm, ⁶⁶Ga, ⁷²Ga, ¹⁴⁶Gd, ¹⁵³Gd, ⁶⁸Ge, ¹⁷⁰Hf, ¹⁷¹Hf, ¹⁹³Hg, ¹⁹³mHg, ¹⁶⁰mHo, ¹³⁰I, ¹³¹I, ¹³⁵I, ¹¹⁴mIn, ¹⁸⁵Ir, ⁴²K, ⁴³K, ⁷⁶Kr, ⁷⁹Kr, ⁸¹mKr, ¹³²La, ²⁶²Lr, ¹⁶⁹Lu, ¹⁷⁴mLu, ¹⁷⁶mLu, ²⁵⁷Md, ²⁶⁰Md, ²⁸Mg, ⁵²Mn, ⁹⁰Mo, ²⁴Na, ⁹⁵Nb, ¹³⁸Nd, ⁵⁷Ni, ⁶⁶Ni, ²³⁴Np, ¹⁵O, ¹⁸²Os, ¹⁸⁹mOs, ¹⁹¹Os, ³²p, ²⁰¹Pb, ¹⁰¹Pd, ¹⁴³Pr, ¹⁹¹Pt, ²⁴³Pu, ²²⁵Ra, ⁸¹Rb, ¹⁸⁸Re, ¹⁰⁵Rh, ²¹¹ Rn, ¹⁰³Ru, ³⁵S, ⁴⁴Sc, ⁷²Se, ¹⁵³Sm, ¹²⁵Sn, ⁹¹Sr, ¹⁷³Ta, ¹⁵⁴Tb, ¹²⁷Te, ²³⁴Th, ⁴⁵Ti, ¹⁶⁶Tm ²³⁰U, ²³⁷U, ²⁴⁰U, ⁴⁸V, ¹⁷⁸W, ¹⁸¹W, ¹⁸⁸W, ¹²⁵Xe, ¹²⁷Xe, ¹³³Xe, ¹³³mXe, ¹³⁵Xe, ⁸⁵mY, ⁸⁶Y, ⁹⁰Y, ⁹³Y, ¹⁶⁹Yb, ¹⁷⁵Yb, ⁶⁵Zn, ⁷¹mZn, ⁸⁶Zr, ⁹⁵Zr, or ⁹⁷Zr.

39. The modified antibody or antigen binding fragment of claim 30, wherein the nanoparticle comprises a metal nanoparticle, liposome, a polymer nanoparticle, or a lipid nanoparticle.

40. The modified antibody or antigen binding fragment of claim 30, wherein the bead comprises a magnetic or a paramagnetic bead.

41. The modified antibody or antigen binding fragment of claim 30, wherein the modified antibody or antigen binding fragment thereof and the secondary binding domain form an antibody with multiple binding domains.

42. The modified antibody or antigen binding fragment of claim 41, wherein the antibody with multiple binding domains comprises binds one epitope, two epitopes, three epitopes, or four epitopes.

43. The modified antibody or antigen binding fragment of claim 41, wherein the antibody with multiple binding domains comprises a bispecific antibody, a trispecific antibody, or a tetraspecific antibody.

44. The modified antibody or antigen binding fragment of claim 30, wherein the secondary binding domain binds an immune cell activating epitope.

45. The modified antibody or antigen binding fragment of claim 44, wherein the secondary binding domain binds a T cell activating epitope or an NK cell activating epitope.

46. The modified antibody or antigen binding fragment of claim 45, wherein the T cell activating epitope comprises CD3, CD28, 4-11BB, or CD27.

47. The modified antibody or antigen binding fragment of claim 45, wherein the NK cell activating epitope comprises CD8, CD16, NKG2A, NKG2D, KIR2DL1, KIR2DL2/3, KIR2DL4, KIR3DL1, NKp44, or KLRG1.

48. A codon-optimized nucleotide sequence encoding the modified antibody or antigen binding fragment of claim 1.

49. The codon-optimized nucleotide sequence of claim 48, comprising a variable heavy chain encoding sequence as set forth in SEQ ID NO: 22, a variable light chain encoding sequence as set forth in SEQ ID NO: 144, and a sequence encoding a linker.

50. The codon-optimized nucleotide sequence of claim 49, wherein the sequence encoding the linker comprises the sequence as set forth in SEQ ID NO: 78.

51. The codon-optimized nucleotide sequence of claim 49, linked to a sequence encoding a viral structural protein with a sequence encoding a second linker.

52. The codon-optimized nucleotide sequence of claim 51, wherein the viral structural protein comprises measles virus hemagglutinin (H), measles virus fusion (F), vesicular stomatis virus G (VSV-G), influenza hemagglutinin, ebolavirus glycoprotein (GP)1 or GP2, coronavirus spike, lassa virus glycoprotein precursor (GP) or spike, nipah virus NiV-G or NiV-F, rabies virus glycoprotein (G), respiratory syncytial virus fusion protein (F) or attachment protein (G), human immunodeficiency virus (HIV) gp41, HIV gp120, paramyxovirus F protein, filovirus GP2 protein, flavivirus E protein, alphavirus E1 protein, herpes simplex virus gH or gB protein, or arenavirus G2 protein.

53. The codon-optimized nucleotide sequence of claim 51, wherein the viral structural protein comprises measles virus hemagglutinin.

54. The codon-optimized nucleotide sequence of claim 51, wherein the viral structural protein comprises VSV-G.

55. The codon-optimized nucleotide sequence of claim 51, wherein the sequence encoding a viral structural protein comprises the sequence as set forth in SEQ ID NO: 161 or SEQ ID NO: 162 or a sequence having at least 95% sequence identity to a sequence as set forth in SEQ ID NO: 161 or SEQ ID NO: 162.

56. The codon-optimized nucleotide sequence of claim 51, wherein the sequence encoding the second linker comprises the sequence as set forth in SEQ ID NO: 64, SEQ ID NO: 66, SEQ ID NO: 78, SEQ ID NO: 164, or SEQ ID NO: 165 or a sequence having at least 95% sequence identity to a sequence as set forth in SEQ ID NO: 64, SEQ ID NO: 66, SEQ ID NO: 78, SEQ ID NO: 164, or SEQ ID NO: 165.

57. A composition comprising an antibody or antigen binding fragment of claim 1 and a pharmaceutically acceptable carrier.

58. A pseudotyped viral vector comprising an antibody or antigen binding fragment of claim 1 linked to a viral structural protein.

59. The pseudotyped viral vector of claim 58, wherein the viral structural protein comprises measles virus hemagglutinin (H), measles virus fusion (F), vesicular stomatis virus G (VSV-G), influenza hemagglutinin, ebolavirus glycoprotein (GP)1 or GP2, coronavirus spike, lassa virus glycoprotein precursor (GP) or spike, nipah virus NiV-G or NiV-F, rabies virus glycoprotein (G), respiratory syncytial virus fusion protein (F) or attachment protein (G), human immunodeficiency virus (HIV) gp41, HIV gp120, paramyxovirus F protein, filovirus GP2 protein, flavivirus E protein, alphavirus E1 protein, herpes simplex virus gH or gB protein, or arenavirus G2 protein.

60. The pseudotyped viral vector of claim 58, wherein the viral structural protein comprises a measles virus hemagglutinin.

61. The pseudotyped viral vector of claim 58, wherein the viral structural protein comprises VSV-G.

62. The pseudotyped viral vector of claim 58, wherein the viral vector is a lentiviral vector, a retroviral vector, an adenoviral vector, or adeno-associated viral (AAV) vector.

63. A pseudotyped viral vector expressing a recombinant protein encoded by the codon-optimized nucleotide sequence of claim 48.

64. The pseudotyped viral vector of claim 63, wherein the viral vector comprises a lentiviral vector, a retroviral vector, or an adenoviral vector.

65. Use of an antibody or antigen binding fragment of claim 1 to target CD90+ cells for delivery of genetic engineering components.

66. The use of claim 65 wherein the genetic engineering components provide a therapeutic gene to the CD90+ cell.

67. The use of claim 65, wherein the genetic engineering components insert or alter a gene selected from ABCA3, ABCD1, Akt, amyloid beta precursor protein (APP), angiopoietin 1 (Ang1), angiotensin-converting enzyme 2 (ACE2), antibodies to CD4, antibodies to CD5, antibodies to CD7, antibodies to CD52, etc., antibodies to IL1, antibodies to IL2, antibodies to IL6, antibodies to TCR specifically present on autoreactive T cells, antibodies to TNF, arylsulfatase A, Bcl-2, brain derived neurotrophic factor (BDNF), cerebral dopamine neurotrophic factor (CDNF), C1q/tumor necrosis factor-related protein-3 (CTRP3), C90RF72, C-C Motif Chemokine Receptor 1 (CCR1), CCR2, chemokine receptor 2 (CXCR2), CXCR4, CXCR7, ciliary neurotrophic factor (CNTF), CLN3, connexin 43 (Cx43), Csx/Nkx 2.5, CTLA, cystic fibrosis transmembrane conductance regulator (CFTR), Cytochrome b5 reductase 3 (CYB5R3), DRB1*1501/DQB1*0602, Dyskerin Pseudouridine Synthase (DKC1), dystrophin, E2F4, endothelial nitric oxide synthase (eNOS), erythropoietin (EPO), extracellular regulating kinase 1/2, F8 (coagulation factor VIII), F9 (coagulation factor IX), Fanconi anaemia complementation group (FANC) family genes, Fas L, fibroblast growth factor-2 (FGF-2), fibroblast growth factor 4 (FGF4), Follistatin-like 1, forkhead box protein (Foxa2), fused in sarcoma (FUS), GATA1, GATA-4, glial cell line-derived neurotrophic factor (GDNF), globin family genes, granulocyte colony-stimulating factor (G-CSF), HBB, heme oxygenase-1 (HO-1), hepatocyte growth factor (HGF), hepatocyte nuclear factor 4α (HNF4α), hypoxia-inducible factor 1α (HIF-1α), insulin-like growth factor (IGF)-1, Intercellular Adhesion Molecule (ICAM)-1, interferon-beta (IFN-β), integrin α4, interleukin-1 receptor antagonist (IL-1Ra), interleukin 4 (IL4), IL10, IL12, IL13, IL-33, Islet-1, Klotho gene, let-7d, leucine-rich repeat kinase 2 (LRRK2), lipocalin 2 (Len2), Mash1, microRNA-1 (miR-1), miR-16-5p, miR-21, miR-25, miR-30b-3p, miR-34, miR-101-3p, miR-124, mIR-126, miR-133, miR-133b, mIR-181a, miR-199a, miR-199a-3p, miR-211, miR-705, nerve growth factor (NGF), neuregulin 4 (Nrg4), neurotrophin-3 (NT3), NLX2.1, Notch ligand Delta-like-4, Notch1 receptor, nuclear factor erythroid-derived 2-like 2 (Nrf2), orphan receptor tyrosine kinase 2 (ROR2), P53, p130, Parkinson disease 2 (PARK2), PARK7, PARKIN, phox, platelet-derived growth factor (PDGF), presenilin 1 (PSEN1), PSEN2, protein tyrosine phosphatase, non-receptor type 22 (PTPN22), PTEN-induced kinase 1 (PINK1), pyruvate kinase, ribosomal protein genes, secreted Klotho protein (SKL), sirtuin 1, soluble CD40, soluble interleukin 1 receptor II (sIL1RII), sIL1RI, soluble TNF alpha receptor I (sTNFRI), sTNFRII, somatostatin 2 (sST2), sonic hedgehog (Shh), superoxide dismutase 1 (SOD1), surfactant protein B (SFTPB), SFTPC, synuclein alpha (SNCA), TAR DNA-binding protein 43 (TDP43), telomerase reverse transcriptase (TERT), telomerase RNA component (TERC), TERF interacting nuclear factor 2 (TINF2), TNF-related apoptosis-inducing ligand (TRAIL), transforming growth factor P1 (TGF-β1), type 2 angiotensin II receptor (AT2R), tyrosine kinase receptor type 3 (TrkC), ubiquilin 2, vascular endothelial growth factor (VEGF), WASP Actin nucleation promoting factor (WAS), or Wnt/β-catenin.

68. The use of claim 65, wherein the CD90+ cells are hematopoietic stem cells or mesenchymal stem/stromal cells.

69. The use of claim 65, wherein the genetic engineering components comprise naked DNA, naked mRNA, guideRNA, a base editor, a clustered regularly interspaced short palindromic repeats (CRISPR) nuclease, a zinc finger (ZFN), a tal effector nuclease (TALEN), a meganucleases or a meganuclease-TALEN fusion (MegaTALE).
